(19) United States
(12) Patent Application Publication
(10) Pub. No.: US 2025/0388959 A1
(43) Pub. Date: Dec. 25, 2025

(54) MULTI-MODALITY PLATFORM IMMUNOTHERAPY AND TUMOR-SPECIFIC T CELLS

(71) Applicant: CelestraBio, Inc., Princeton, NJ (US)

(72) Inventor: Ruey-min Lee, Short Hills, NJ (US)

(73) Assignee: CelestraBio, Inc., Princeton, NJ (US)

(21) Appl. No.: 19/243,851

(22) Filed: Jun. 20, 2025

Related U.S. Application Data
(60) Provisional application No. 63/661,983, filed on Jun. 20, 2024.
(60) Provisional application No. 63/744,413, filed on Jan. 13, 2025.

(51) Int. Cl. C12Q 1/686; A61K 35/14; A61K 39/00; A61K 40/10; A61K 40/42; A61K 45/06; A61P 35/00; C12N 5/00; C12N 5/0781; C12N 5/0783; C12N 15/10; C12Q 1/6855; C12Q 1/6869; C40B 40/06; C40B 50/00

(52) U.S. Cl. CPC C12Q 1/686; A61K 35/14; A61K 39/00; A61K 40/10; A61K 40/42; A61K 45/06; A61P 35/00; C12N 5/0087; C12N 5/0635; C12N 5/0636; C12N 5/0646; C12N 15/1003; C12Q 1/6855; C12Q 1/6869; C40B 40/06; C40B 50/00

(57) ABSTRACT

The present disclosure provides an autologous cell therapy for treating a cancer. Transarterial tirapazamine embolization (TATE) therapy induces tumor necrosis, which, in combination with anti-PD-1 therapy, enhances the efficacy of anti-PD-1 through TATE-induced expansion of anti-tumor T cells activated by the anti-PD-1 antibody. PBMCs collected from TATE and PD-1-treated patients for RNA and DNA extraction and next generation sequencing (NGS) analysis of complementarity region-3 of the TCR from T cell populations in the PBMCs show that clonal expansion of anti-tumor specific T cell receptors (TCRs) occurs. Expansion of the PBMC population for administration to a cancer patient preferentially expands the population of effector T cells targeting the tumor cells without a need for genetic manipulation.

### CROSS-REFERENCE TO RELATED APPLICATIONS

This application claims the benefit of priority to U.S. provisional application 63/661,983 (filed Jun. 20, 2024) and to U.S. provisional application 63/744,413 (filed Jan. 13, 2025). The content of each of these applications is incorporated herein by reference.

### FIELD OF THE INVENTION

This application relates to an autologous cellular immunotherapy for treating a cancer.

### BACKGROUND OF THE INVENTION

### Immune Mechanisms Implicated in the Body's Response to Cancer

The human immune system is a complex arrangement of cells and molecules that maintain immune homeostasis to preserve the integrity of the organism by elimination of all elements judged to be dangerous. Responses in the immune system may generally be divided into two arms, referred to as “innate immunity” and “adaptive immunity.” The two arms of immunity do not operate independently of each other but rather work together to elicit effective immune responses.

### Tumor-Rejection Antigens

Generally, tumor rejection antigens recognized by the immune system are peptides of tumor-cell proteins that are presented to T cells on MHC molecules. These peptides can become the targets of a tumor specific T cell response even though they can also be present on normal tissues.

There are several categories of tumor rejection antigens. One consists of tumor-specific antigens that result from point mutations or gene arrangements that affect a particular gene product. The mutated peptide is often referred to as a neoepitope since they are newly immunogenic versions of normal proteins. A second category is proteins encoded by genes that are normally expressed in male germ cells in the testis, or so-called cancer-testis antigens. Male germ cells do not express MHC molecules, and therefore peptides from these molecules are not normally presented to T lymphocytes. A third category is differentiation antigens encoded by genes that are expressed only in particular types of categories. A fourth category consists of antigens that are strongly overexpressed in tumor cells compared with their normal counterparts (e.g., Her-2/neu, a receptor tyrosine kinase homologous to EGF receptor). A fifth category is molecules that display abnormal post-translational modifications (e.g., underglycosylated mucin, MUC-1, which is expressed by breast and pancreatic cancers). Sixth is novel proteins generated when one or more introns are retained in the mRNA transcribed from a gene. Seven are proteins encoded by viral oncogenes. Other potential tumor rejection antigens include the products of mutated cellular oncogenes or tumor suppressors, such as Ras and p53, and fusion proteins that result from chromosomal translocation. [[[Janeway's Immunology, 9th Ed. (2017) Garland Science, New York, at 717].

Tumors can avoid stimulating an immune response or can evade it when it occurs by means of several mechanisms. Spontaneous tumors may initially lack mutations that produce new tumor-specific antigens that elicit T cell responses. Even when a tumor-specific antigen is expressed and is taken up and presented by antigen-presenting cells, if co-stimulatory signals are absent, the APC will tend to tolerize any antigen-specific naïve T cells rather than activating them. [Janeway's Immunology, 9th Ed. (2017) Garland Science, New York at 718].

Cellular transformation frequently is associated with the induction of MHC class Ib proteins (such as MIC-A and MIC-B) that are ligands for NKG2D, thus allowing tumor recognition by NK cells. [Id.]

Since cancer cells tend to be genetically unstable, clones that are not recognized by an immune response may be able to escape elimination. Some tumors lose the expression of a particular MHC class I molecule, perhaps through immune selection by T cells specific for a peptide presented by that MHC class I molecule. In experimental studies, when a tumor loses expression of all MHC class I molecules, it can no longer be recognized by cytotoxic T cells although it might become susceptible to NK cells. [Id.]

Tumors also seem to be able to evade immune attack by creating a microenvironment that is generally immunosuppressive. Many tumors make immunosuppressive cytokines. TGF-β tends to suppress the inflammatory T cell responses and cell-mediated immunity needed to control tumor growth. The microenvironments of some tumors also contain populations of myeloid-derived suppressor cells (MDSCs) which can inhibit T cell activation within the tumor. [[Janeway's Immunology, 9th Ed. (2017) Garland Science, New York at 718-9].

Some tumors express cell-surface proteins that directly inhibit immune responses, e.g., PD-L1, a B7 family member and ligand for the inhibitory receptor PD-1 expressed by activated T cells.

Tumors can produce enzymes that act to suppress local immune responses; the enzyme indoleamine 2,3-diogenase (IDO) catabolizes tryptophan, an essential amino acid in order to produce the immunosuppressive metabolite kynurene.

Tumor cells can produce materials such as collagen in the tumor microenvironment that create a physical barrier to interaction with cells of the immune system. [Janeway's Immunology, 9th Ed. (2017) Garland Science, New York, at 719].

### Chronic Inflammation as a Hallmark of Cancer

Chronic inflammation is a critical hallmark of cancer, with at least 25% of cancers associated with it [Gonzalez, H. et al. Genes & Development (2018) 32: 1267-1284, citing Hussain, S P et al. (2000) Cancer Res 60: 3333-3337; Coussens L M, Werb Z. (2002) Nature 420: 860-867; Beaugerie, L. et al. (2013) Gastroenterology 145: 166-175 e168], and possible underlying causes include microbial infections, autoimmunity, and immune deregulation. Cancer-associated inflammation, which is present at different stages of tumorigenesis, contributes to genomic instability, epigenetic modification, induction of cancer cell proliferation, enhancement of cancer anti-apoptotic pathways, stimulation of angiogenesis, and, eventually, cancer dissemination [Id., citing Hanahan D, Weinberg R A. (2011) Cell 144: 646-674]. Studies during the last two decades have demonstrated that inflammatory immune cells are essential players of cancer-related inflammation.

Aberrant innate and adaptive immune responses contribute to tumorigenesis by selecting aggressive clones, inducing immunosuppression, and stimulating cancer cell proliferation and metastasis [Id., citing P Palucka A K, Coussens L M. (2016) Cell 164: 1233-1247]. During the early stages of tumor development, cytotoxic immune cells such as natural killer (NK) and CD8+ T cells recognize and eliminate the more immunogenic cancer cells [Id., citing Teng, M W et al. (2015) J Clin Invest 125: 3338-3346]. This first phase of elimination selects the proliferation of cancer cell variants that are less immunogenic and therefore invisible to immune detection. As the neoplastic tissue evolves to a clinically detectable tumor, different subsets of inflammatory cells impact tumor fate. For example, high levels of tumor-infiltrated T cells correlate with good prognosis in many solid cancers [Id., citing Clemente, D. et al. (1996) Cancer 77: 1303-1310; Oldford, S A et al. (2006) Int Immunol 18: 1591-1602; Dieu-Nosjean, M C et al. (2008) J Clin Oncol 26: 4410-4417]; on the other hand, high levels of macrophage infiltration correlate with a worse prognosis [Id., citing Zhang, Q W et al. (2012) PLOS One 7: e50946; Mantovani, A. et al. (2017) Trends Immunol 23: 549-555; Gonzalez, H. et al. (2018) FEBS J 285: 654-664].

### Macrophages

Macrophages are innate immune cells that differentiate from circulating classical monocytes after extravasation into tissues. Upon differentiation, macrophages are equipped to sense and respond to infections and tissue injuries, playing a key role in tissue homeostasis and repair [Id., citing Lavin, Y. et al. (2015) Nat Rev Immunol 15: 731-744]. As crucial drivers of chronic cancer-associated inflammation, their involvement has been described in every step of cancer progression, from early neoplastic transformation throughout metastatic progression to therapy resistance [Id., citing Noy R, Pollard J W. (2014) Immunity 41: 49-61; Kitamura, T. et al. (2015) Nat Rev Immunol 15: 73-86; Gonzalez, H. et al. (2018) FEBS J 285: 654-664]. In oncological patients and preclinical experimental models, high-grade tumor-associated macrophages (TAMs) correlate with poor prognosis and reduced overall survival [Id., citing Zhang Q W et al. (2012) PLOS One 7: e50946; Noy R, Pollard J W. (2014) Immunity 41: 49-61].

Activated macrophages are referred to as either proinflammatory (“M1 type,” driven by LPS and IFNγ) or anti-inflammatory (“M2-type,” driven by IL-4 or IL-13) [Id., citing Mantovani, A. et al. (2002). Trends Immunol 23: 549-555]. During carcinogenesis, anti-tumor macrophages display an M1-like polarization that plays a relevant role in the elimination of more immunogenic cancer cells. As the tumor progresses, the tumor microenvironment (TME) elicits an M2-like polarization of TAMs that is protumorigenic [Id., citing Mantovani, A. et al. (2017) Nat Rev Clin Oncol 14: 399-416]. TAMs promote tumor progression in different ways, such as stimulating angiogenesis and lymphangiogenesis, stimulating both cancer cell proliferation and epithelial-mesenchymal transition, limiting the efficacy of therapies, remodeling the ECM, promoting metastasis, and inducing immunosuppression of anti-tumor effector immune cells [Id., citing DeNardo, G. et al. (2011) Cancer Discov. 1: 54-67; Qian, B Z et al. (2015) J Exp Med 212: 1433-1448; Mantovani, A. et al. (2017) Nat Rev Clin Oncol 14: 399-416]. Accordingly, TAMs secrete cytokines such as IL-10 [Id., citing Ng, T H et al. (2013) Front Immunol 4: 12] and TGF-β [McIntire, R H et al. (2004) J Leukoc Biol 76: 1220-1228] that induce immunosuppression, impairing the activity of effector T cells and inhibition of dendritic cell (DC) maturation [Id., citing Rubtsov, Y P et al. (2008) Immunity 28: 546-558]. TAMs also directly stimulate cancer cell proliferation through the secretion of epidermal growth factor (EGF) [Id., citing O'Sullivan, C. et al. (1993) Lancet 342: 148-149]., promote tumor angiogenesis by vascular EGF (VEGF) secretion [Id., citing Shojaei, F. et al. (2008) Trends Cell Biol 18: 372-378], and remodel the ECM by secreting metalloproteinases (MMPs) [Id., citing Kessenbrock, K. et al. (2010) Cell 141: 52-67]. For example, macrophage-derived MMP-9 promotes tumorigenesis and angiogenesis [Id., citing Huang, S. et al. (2002) J Natl Cancer Inst 94: 1134-1142].

Although TAMs mostly play protumorigenic roles, they can also sometimes exert anti-tumoral roles. For example, nonclassical NR4A1+ patrolling monocytes that, in steady state conditions, are located in the microvasculature of different organs inhibit lung metastasis in MMTV-PyMT mice by direct induction of NK cell recruitment to the metastatic site [Id., citing Hanna, R N et al. (2015) Science 350: 985-990]. Additionally, TAMs mediate the efficacy of the anti-tumor and anti-metastatic effects of the histone deacetylase inhibitor TMP195, which reprograms TAMs to a highly phagocytic phenotype [Id., citing Guerriero, J L et al. (2017) Nature 543: 428-432].

Although the M1-like/M2-like paradigm has proved to be useful, transcriptomic analysis suggests that it is likely that a spectrum of differentiated TAMs/metastasis-associated macrophages (MAMs) exists and that the current model is oversimplified [Id., citing Xue, J. et al. (2014) Cancer Cell 32: 169-184.e7].

### Neutrophils

Neutrophils are recognized as key players during inflammation. They are among the first immune cells to be recruited to damaged tissue, where they can eliminate pathogens and modulate inflammation by mechanisms such as phagocytosis, secretion of antibacterial proteins, deposit of neutrophil extracellular traps (NETs), and exocytosis of protease-containing granules [Id., citing Kolaczkowska, E. and Kubes, P. (2013) Nat Rev Immunol 13: 159-175]. In cancer patients, high levels of tumor-associated neutrophils (TANs), high levels of neutrophilia, and/or high neutrophil/lymphocyte ratios have been associated with an adverse prognosis in different malignances [Id., citing Keizman, D. et al. (2012) Eur J Cancer 48: 202-208; Donskov, F. (2013) Semin Cancer Biol 23: 200-207]. Similar to the M1/M2 phenotype of macrophages, it has been proposed that TANs exist in two polarization states, called “N1” and “N2,” to describe protumor and anti-tumor populations, respectively [Id., citing Fridlender, Z G et al. (2009) Cancer Cell 16: 183-194]. This paradigm is still a matter of debate due to the lack of specific markers to identify these two populations. However, it is clear that TANs display functional heterogeneity. The recruitment of TANs to the TME is thought be mediated mainly by CXCR2 ligands such as CXCL1, CXCL2 and CXCL5 [Id., citing Jamieson, T. et al. (2012) J Clin Invest 122: 3127-3144; Katoh, H. et al. (2013) Cancer Cell 24: 631-644], secreted by cancer and stromal cells; TGF-β has also been associated with recruitment and reprogramming to protumor TANs [Id., citing Fridlender, Z G et al. (2009) Cancer Cell 16: 183-194].

In xenograft models of melanoma and lung cancer, TANs expressing hepatocyte growth factor receptor (c-MET) play important anti-tumor and anti-metastatic roles. c-MET expression is induced by tumor-derived tumor necrosis factor-α (TNFα) [Id., citing Finisguerra, V. et al. (2015) Nature 522: 349-53], and it is likely that NK and effector T cells are a source of TNF-α within the TME. Similarly, in human colorectal cancer, high levels of CD66b+ TANs have been associated with better prognosis by enhancing the tumoricidal capacity of CD8+ T cells ([Id., citing Governa, V. et al. (2017) Clin Cancer Res 23: 3847-3858]. Neutrophils also exert a tumoricidal function during radiotherapy. As they are rapidly and transiently recruited to tumor sites in syngeneic xenograft breast cancer models, the concurrent administration of granulocyte colony-stimulating factor (G-CSF) enhances radiotherapy effectiveness ([Id., citing Takeshima, T. et al. (2016) Proc Natl Acad. Sci 113: 11300-11305].

In contrast, TANs are thought to contribute to nascent inflammation during cancer initiation and progression. In a Kras-driven lung adenocarcinoma mouse model, IL-17-responsive TANs promote tumor growth [Id., citing Chang, S H et al. (2014) Proc Natl Acad. Sci 111: 5664-5669]. Also, neutrophil elastase acts as a potent elastolytic enzyme that, when secreted in a site of inflammation, promotes tumor cell invasion, angiogenesis, and cancer cell proliferation [Id., citing Houghton, A M et al. (2010) Nat Med 16: 219-223; Gong, L. et al. (2013) Mol Cancer 12: 154]. Moreover, TANs contribute to tumor angiogenesis by the secretion of MMP9 and VEGF in genetic mouse models of pancreatic and colon cancer [Id., citing Bergers, G. et al. (2000) Nat Cell Biol. 2: 737-744; Shojaei, F. et al. (2007) Nature 450: 825-831; Shojaei, F. et al. (2008) Trends Cell Biol 18: 372-378]. In gastric cancer, TANs induce direct immunosuppression in T cells by PD-L1 expression induced by tumor-derived granulocyte macrophage-CSF (GM-CSF) [Id., citing Wang, T T et al. (2017) Gut 66: 1900-1911]. A population of cells phenotypically and morphologically similar to neutrophils, called polymorphonuclear myeloid-derived suppressor cells (PMN-MDSCs), has been identified in cancer patients and preclinical models [Id., citing Gabrilovich, D I (2017) Nat Med 2: 1096-1103]. The presence of PMN-MDSCs in tumors is associated with induction of chronic inflammation and antigen-specific tolerance by T cells [Id., citing Marigo, I. et al. (2010) Immunity 32: 790-802].

Neutrophils have been proposed to be pioneer cells in the lung premetastatic niche, supporting the arrival of disseminated cells in the MMTV-PyMT model [Id., citing Wculek S K, Malanchi I. (2015) Nature 528: 413-417]. Moreover, in response to the secretion of IL-7 by γδ T cells, neutrophils are recruited to the lungs, where they support the survival and proliferation of disseminated cancer cells by suppressing effector CD8+ T cells [Id., citing Coffelt, S B et al. (2015) Nature 522: 345-348]. This prometastatic motif is also seen during liver metastasis in the KPC model of pancreatic cancer [Id., citing Steele, C W et al. (2016) Cancer Cell 29: 832-845]. These results demonstrate that the protumor and anti-tumor functions of TANs are highly context-dependent and likely depend on immune crosstalk with other tumor-associated immune cells.

In recent years, the presence of NETs in the tumor microenvironment (TME) has been linked to cancer progression in animal models and cancer patients [Id., citing Cools-Lartigue, J. et al. (2013) J Clin Invest 123: 3446-3458, Cools-Lartigue, J. et al. (2014) Cell Mol Life Sci 71: 4179-4194; Tohme, S. et al. (2016) Cancer Res 76: 1367-1380]. NETs are extracellular networks released by neutrophils-composed mostly of chromatin, proteases (such as elastase, cathepsin G, and MMP9), and intracellular proteins—that immobilize pathogens to facilitate their subsequent elimination [Id., citing Papayannopoulos, V. (2018) Nat Rev Immunol 18: 134-147]. An increase in NET formation has been correlated with progression to metastatic disease in colorectal cancer patients after surgery [Id., citing Tohme, S. et al. (2016) Cancer Res 76: 1367-1380]. Additionally, NETs trap circulating cancer cells, increasing the adhesion within hepatic sinusoids, which favors extravasation and parenchyma colonization [Id., citing Cools-Lartigue, J. et al. (2013) J Clin Invest 123: 3446-3458]. In breast cancer, NETs accumulate around metastatic cells that have reached the lungs of mice. Notably, targeting NETs in vivo with DNase I-coated particles reduces metastatic burden [Id., citing Park, J. et al. (2016) Sci Transl Med 8: 361ra138]. NETs seem to play a protumor role by the direct activity of NET-derived proteases and also by holding cancer cells in place, likely facilitating the concentration and localization of cancer effectors that result in increased degradation of the ECM, migration, and invasion [Id., citing Cools-Lartigue, J. et al. (2014) Cell Mol Life Sci 71: 4179-4194].

### NK Cells

NK cells are innate immune cells that display rapid and potent cytolytic activity in response to infected or transformed cells [Id., citing Cerwenka A, Lanier L L. (2016) Nat Rev Immunol 16: 112-123]. NK cells have a wide array of inhibitory and stimulatory receptors on their cell surface that are used for immune surveillance. The inhibitory receptors target cancer cells lacking major histocompatibility class I (MHC-I), marking them for programmed cell death [Id., citing Marcus, A. et al. (2014) Adv Immunol 122: 91-128]. In contrast, in healthy cells, the binding of MHC-I molecules to their receptors on NK cells has a profound inhibitory effect on NK cell function [Id., citing Bix, M. et al. (1991) Nature 349: 329-331; Liao, N S et al. (1991) Science 253: 199-202; Colonna, M. et al. (1992) Proc Natl Acad. Sci 89: 7983-7985; Karlhofer, F M et al. (1992) Nature 358: 66-70; Wagtmann, N. et al. (1995) Molecular clones of the p58 NK cell receptor reveal immunoglobulin-related molecules with diversity in both the extra- and intracellular domains. Immunity 2: 439-449; Lanier, L L. (2005) J. Immunol. 174: 6565]. NK cells have a well-documented anti-tumor effect [Id., citing Marcus, A. et al. (2014) Adv Immunol 122: 91-128; Iannello, A. et al. (2016) Curr. Opin. Immunol. 38: 52-58]. In this regard, the presence of NK cell infiltration in colorectal [Id., citing Coca, S. et al. (1997) Cancer 79: 2320-2328] and gastric [Id., citing Ishigami, S. et al. (2000) Cancer 88: 577-583] tumors correlates with a favorable outcome. Hence, there appears to be an intricate link between incipient tumor transformation and the ability of innate immune cells to recognize it. Indeed, in mice, aberrant cell proliferation induces production of the ligand retinoic acid early transcript 1 (RAE1), which is recognized by the stimulatory receptor NKG2D, expressed on NK cells [Id., citing Raulet D H. (2003) Nat Rev Immunol 3: 781-790]. Besides aberrant cell proliferation, DNA damage (Gasser, S. et al. (2005) Nature 436: 1186-1190) and RAS pathway activation [Id., citing Liu, X V et al. (2012) J Immunol 189: 1826-1834] induce production of ligands in tumor cells, which are recognized by NKG2D receptors on NK cells. In line with the important role of NKG2D for immune surveillance, mice deficient in NKG2D receptor are more susceptible to tumor development [Id., citing Guerra, N. et al. (2008). Immunity 28: 571-580]. Besides NKG2D, NK cells have a repertoire of different stimulatory cell surface receptors, which, upon binding to their tumor-derived ligands, activate NK cells [Id., citing Cerwenka, A. et al. (2000) Immunity 12: 721-727; Diefenbach, A., et al. (2000) Nat Immunol 1: 119-126, Diefenbach, A., et al. (2001) Nature 413: 165-171; Raulet D H. (2003). Nat Rev Immunol 3: 781-790]. In a mouse model of hepatic carcinoma, the restoration of the tumor suppressor p53 in cancer cells promotes the elimination of senescent cells [Id., citing Iannello, A. et al. (2013) Curr. Opin. Immunol. 38: 52-58].

NK cells function in controlling cancer progression. Upon activation, NK cells mediate the tumor killing mainly by releasing cytotoxic perforin [Id., citing Voskoboinik, I. et al. (2006) Nat Rev Immunol 6: 940-952] and granzyme, eliminating tumor cells and also triggering apoptotic pathways in tumor cells through the production of TNFα or via direct cell-cell contact through activation of the TRAIL and FASL pathways. Densely granulated NK cells are recruited into large solid tumors by tumor-produced IL-15, where they successfully eliminate established tumors [Id., citing Liu, R B et al. (2012) Cancer Res 72: 1964-1974]. The natural cytotoxicity receptor NKp46 and inhibitory receptor Ly49 on NK cells prevent metastatic outgrowth in melanoma, lung, and fibrosarcoma models [Id., citing Andrews, D M et al. (2012) Nat Immunol 13: 1171-1177; Glasner, A. et al. (2012) J Immunol 188: 2509-2515].

### Dendritic Cells

Dendritic cells are specialized antigen-presenting cells (APCs) that represent the interface between innate and adaptive immunity; they are able to present endogenous and exogenous antigens to T cells in the context of MHC molecules. With the exception of the brain parenchyma, DCs are located in every tissue across the body [Id., citing Mildner A, Jung S. (2014) Immunity 40: 642-656]. During tumor development, DCs prime naïve and memory T cells, and, depending on the inflammatory context and the costimulatory signals, the antigen presentation can result in antigen tolerance or priming and triggering of an effector T-cell response. Tumor-infiltrating DCs have been described in many cancer types [Id., citing Tran Janco, J M et al. (2015) J Immunol 194: 2985-2991; although their activity is necessary to explain the role of T cells during cancer progression, DC involvement in cancer progression remains understudied.

Insights into DC mechanisms are limited. A mouse model of fibrosarcoma lacking CD8a+ DCs shows impaired tumor rejection mediated by CD4+ and CD8+ T cells [Id., citing Hildner, K. et al. (2008) Science 322: 1097-1100]. This anti-tumor effect of CD8a+ DCs priming effector T cells is selectively dependent on type I interferon production [Id., citing Diamond, M S et al. (2011) J Exp Med 208: 1989-2003]. CD103+ DCs have critical roles in tumor antigen presentation in transgenic and xenograft mouse models of melanoma and breast and cervical cancer [Id., citing Broz, M L et al. (2014) Cancer Cell 26: 938; Moynihan, K D et al. (2016) Nat Med 22: 1402-1410; Roberts, E W et al. (2016) Cancer Cell 30: 324-336]. During chemotherapy-induced anti-tumor immune responses, ATP and damage signals released by necrotic cells induce the recruitment of myeloid cells; this is followed by local differentiation to CD11c+ CD11b+Ly6Chi DCs that efficiently engulf tumor antigens in situ and prime the anti-tumor effector T-cell response [Id., citing Ma, Y. et al. (2013) Immunity 38: 729-741]. Mechanistically, the expression of formyl-peptide receptor 1 on DCs favors the recognition and stable interaction with dying cancer cells followed by maturation, engulfing, and antigen presentation in breast and colorectal cancer [Id., citing Vacchelli, E. et al. (2015) Science 350: 972-978]. Intravital imaging analysis identified lung-resident CD103+ DCs as direct suppressors of metastatic melanoma cells [Id., citing Headley, M B et al. (2016) Nature 531: 513-517]. These findings highlight the essential role of DCs.

Several clinical trials in phases I, II, and III tested the use of autologous DCs pulsed with tumor antigens (DC vaccine) to initiate an anti-tumor T-cell response, with promising but limited success, especially in melanoma and prostate cancer [Id., citing Mukherji, B. et al. (1995) Proc Natl Acad Sci 92: 8078-8082; Nestle, F O et al. (1998) Nat Med 4: 328-332; Beer, T M et al. (2011) Clin Cancer Res 17: 4558-4567]. The limitations in the use of DC vaccines include ex vivo manipulation such as antigen loading, which impacts DC function in vivo, and also the lack of deep insights into DC subsets and their functional specialization in cancer [Id., citing Santos P M, Butterfield L H. (2018) J Immunol 200: 443-449]. A phase II study in 39 melanoma patients showed that the combination of an intradermal DC vaccine combined with CTLA-4 blockade resulted in eight complete and seven partial therapeutic responses [Id., citing Wilgenhof, S. et al. (2016) J Clin Oncol 34: 1330-1338].

Overall, DCs play a key role in the priming and consolidation of anti-tumor adaptive immune response; a better understanding of such mechanisms will shed light on how the anti-tumor T-cell attack fails to eliminate and contain the tumor development. In this sense, massive parallel single-cell analysis in early lung adenocarcinoma (stage I) has identified a selective depletion of CD141+ DCs (compared with normal lung tissue) that correlates with impaired NK and T-cell activity, which favors tumor progression [Id., citing Lavin, Y. et al. (2017) Cell 169: 750-765.e17]. It has been shown that in melanoma, breast, and colorectal mouse models, tumor cells impair DC recruitment to TME by secretion of prostaglandin E2, which impairs the function of tumor-associated NK cells and results in impaired NK cell-dependent DC recruitment [Id., citing Bottcher, J P et al. (2018) Cell 172: 1022-1037.e14].

### T Cells

T cells are components of the adaptive immune system that act as orchestrators and effectors of immunity. Depending on the immunological context, T cells can acquire functional and effector phenotypes whose activity has direct inflammatory or anti-inflammatory consequences [Id., citing Speiser, D E et al. (2016) Nat Rev Immunol 16: 599-611]. As the second most frequent immune cell type found in human tumors besides TAMs, T cells are extensively studied in diverse cancer types [Id., citing Speiser, D E et al. (2016) Nat Rev Immunol 16: 599-611; Donadon, M. et al. (2017) J Gastrointest Surg 21: 1226-1236]. During the early stages of tumor initiation, if enough immunogenic antigens are produced, naïve T cells will be primed in the draining lymph nodes, followed by their concomitant activation and migration to the TME. From there, they mount a protective effector immune response, eliminating immunogenic cancer cells. Histopathological analyses of human tumors show that tumor-associated T cells extend beyond the invasive edge of the tumor and also predominate in its hypoxic core [Id., citing Halama, N. et al. (2011) Cancer Res 71: 5670-5677; Kirilovsky, A. et al. (2016) Int Immunol 28: 373-382]. A high level of T-cell infiltration in tumors is associated with a favorable prognosis in melanoma [Id., citing Clemente, C G et al. (1996) Cancer 77: 1303-1310] and breast [Id., citing Oldford, S A et al. (2006) Int Immunol 18: 1591-1602], lung [Id., citing Dieu-Nosjean, M C et al. (2008) J Clin Oncol 26: 4410-4417], ovarian [Id., citing Kusuda, T. et al. (2005) Oncol Rep 13: 1153-1158], colorectal [Id., citing Tosolini, M. et al. (2011) Cancer Res 71: 1263-1271, renal [Id., citing Kondo, T. et al. (2006) Cancer Sci 97: 780-786, prostate [Id., citing Vesalainen, S. et al. (1994) Eur J Cancer 30A: 1797-1803, and gastric [Id., citing Ubukata, H. et al. (2010) J Surg Oncol 102: 742-747; Fridman, W H et al. (2012) Nat Rev Cancer 12: 298-306; Kitamura, T. et al. (2015) Nat Rev Immunol 15: 73-86] cancer.

CD8+ T cells are the most prominent anti-tumor cells. Upon priming and activation by APCs, the CD8+ T cells differentiate into cytotoxic T lymphocytes (CTLs) and, through the exocytosis of perforin- and granzyme-containing granules, exert an efficient anti-tumoral attack, resulting in the direct destruction of target cells [Id., citing Hanson, H L et al. (2000) Immunity 13: 265-276; Matsushita, H. et al. (2012) Nature 482: 400-404]. Meanwhile, the CD4+ T helper 1 (Th-1)-mediated anti-tumoral response-through secretion of high amounts of proinflammatory cytokines such as IL-2, TNF-α, and IFN-γ-promotes not only T-cell priming and activation and CTL cytotoxicity but also the anti-tumoral activity of macrophages and NK cells and an overall increase in the presentation of tumor antigens [Id., citing Kalams S A, Walker B D. (1998) J Exp Med 188: 2199-2204; Pardoll D M, Topalian S L. (1998). Curr. Opin. Immunol. 10: 588-594; Shankaran, V. et al. (2001) Nature 410: 1107-1111]. The presence of tumor-infiltrating CD8+ T cells and Th-1 cytokines in tumors correlates with a favorable prognosis in terms of overall survival and a disease-free survival in many malignancies [Fridman, W H et al. (2012) Nat Rev Cancer 12: 298-306].

Preclinical investigations in patients and mouse models suggest that cancer cells exploit the immunosuppressive properties of T cells while impairing the effector functions of anti-tumor T cells, such as their ability to infiltrate tumors and their survival, proliferation, and cytotoxicity [Id., citing Grivennikov, S I et al. (2010) Cell 140: 883-899]. The antigen-dependent nature of the effector T cells implies that the effectiveness of the anti-tumor T-cell immune response depends on both the ability of the tumor antigen to induce an immune response (immunogenic) and the presence—or absence—of inhibitory signals that can impair T cell functions [Id., citing Speiser, D E et al. (2016) Nat Rev Immunol 16: 599-611]. Accordingly, it is widely accepted that, in a T-cell-dependent process, most neoplastic cells expressing highly immunogenic antigens will be recognized and killed during the early stages of tumor development [Id., citing Matsushita, H. et al. (2012) Nature 482: 400-404]. The less immunogenic cancer cells escape the immune control of T cells and survive, a process termed cancer immune editing [Id., citing Teng, M W et al. (2015) J Clin Invest 125: 3338-3346]. The final outcome is that the surviving cancer cells adopt an immune-resistant phenotype. In parallel, during tumor development, cancer cells evolve mechanisms that mimic peripheral tolerance and are able to prevent the local cytotoxic response of effector T cells as well as those of other cells, such as TAMs, NK cells, and TANs [Id., citing Palucka A K, Coussens L M. (2016) Cell 164: 1233-47].

During immune homeostasis, a crucial mechanism of peripheral tolerance is the regulation of effector T-cell response via immune checkpoints on CTLs and activated CD4+ T cells to protect tissue from inflammatory damage. The two better described checkpoint molecules CTLA-4 and PD-1, act as negative regulators of T-cell function and have been associated with immune evasion in cancer [Id., citing Pardoll, DM (2012) Nat Rev Cancer 12: 252-264]. The involvement of CTLA-4 signaling in cancer has been described in melanoma ([Id., citing Bouwhuis, M G et al. (2010) Cancer Immunol Immunother 59: 303-312] and lung ([Id., citing Khaghanzadeh, N. et al. (2010) Cancer Genet Cytogenet 196: 171-174; Erfani, N. et al. Lung Cancer (2012) 77 (2): 306-11], gastric [Id., citing Hadinia, A. et al. (2007) J Gastroenterol Hepatol 22: 2283-2287], and colorectal [Id., citing Hadinia, A. et al. (2007) J Gastroenterol Hepatol 22: 2283-2287; Dilmec, F. et al. (2008) Int J Immunogenet 35: 317-321] cancer. Furthermore, the engagement of PD1 with its coreceptor, PDL-1 (expressed by other immune cells, mesenchymal cells, vascular cells, and cancer cells), results in the down-regulation of T-cell activity, which inhibits their anti-tumor activities such as T-cell migration, proliferation, secretion of cytotoxic mediators, and restriction of cell killing [Id., citing Topalian, S L et al. (2015) Cancer Cell 27: 450-461]. The use of immune checkpoint inhibitors such as anti-PD1 (pembrolizumab and nivolumab), anti-PD-L1 (MPDL3280A), and anti-CTLA4 (ipilimumab) has had success enhancing the effector anti-tumor response in different malignancies [Id., citing Gotwals, P. et al. (2017) Nat Rev Cancer 17: 286-301], especially in melanoma and lung cancer [Id., citing Hamid, O. et al. (2013) N Engl J Med 369: 134-144; Herbst, R S et al. (2014) Nature 515: 563-567; Topalian, S L et al. (2015) Cancer Cell 27: 450-461].

As the tumor grows and the TME changes, new antigens are produced, and the ability of the immune system to prime new repertoires of T cells and direct them toward the tumor changes, thus altering the efficacy of tumor containment. As the immune system functions to stall tumor growth, cancer cells and the TME simultaneously suppress anti-tumor function by engaging immune checkpoints and the recruitment of regulatory CD4+ T cells (Tregs). Tregs are responsible for suppressing the priming, activation, and cytotoxicity of other effector immune cells, such as Th1 CD4 T cells, CTLs, macrophages, NK cells, and neutrophils [Id., citing Ward-Hartstonge K A, Kemp R A. (2017). Clin. Transl. Immunology 6: e154]. The Treg-mediated immunosuppression is orchestrated by contact-dependent mechanisms such as the expression of PDL-1, LAG-3, CD39/73, CTLA4, or PD1, with the latter two even enhancing suppressive activity ([Id., citing Walker L S, Sansom D M. (2015) Trends Immunol 36: 63-70], and by contact-independent mechanisms, which involve the sequestration of IL-2 and production of immune-suppressive molecules such as IL-10, TGF-β, prostaglandin E2, adenosine, and galectin-1 [Id., citing Francisco, L M et al. (2009) J Exp Med 206: 3015-3029; Campbell, DJ (2015) Eur. J Immunol 195: 2507-2513]. In squamous cell carcinoma, the inhibition of focal adhesion kinase (FAK)—a cell contact-independent mechanism—results in CCL5 secretion by cancer cells that induces the recruitment of Tregs to the tumor site, where they suppress the cytotoxic anti-tumor CD8+ T cells ([Id., citing Serrels, A. et al. (2015) Cell 163: 160-173]. In breast and lung adenocarcinoma, Tregs suppress T-cell activation and the anti-tumor immune response in tumor-associated tertiary structures. Specific Treg depletion results in tumor cell death and increased production of IFN-γ [Id., citing Bos, P D et al. (2013) J Exp Med 210: 2435-2466; Joshi, N S et al. (2015) Immunity 43: 579-590]. Infiltration of Tregs in breast cancer was correlated with worse patient outcome [Id., citing Allaoui, R. et al. (2017) Cancer Biomark 20: 395-409].

In metastasis, CTLs exert an anti-metastatic effect in bone metastasis [Id., citing Bidwell, B N et al. (2012) Nat Med 18: 1224-1231], while prospective analyses of lung and breast cancer patients established an opposite correlation between the level of circulating cancer cells and T cells in peripheral blood [Id., citing Mego, M et al. (2016) J Cancer 7: 1095-1104; Sun, W W et al. (2017) Onco Targets Ther 10: 2413-2424].

These data extend to clinical trials reporting the therapeutic efficacy of immune checkpoint inhibition in metastatic carcinomas [Id., citing Di Giacomo, A M et al. (2012) Lancet Oncol 13: 879-886; Queirolo, P. et al. (2014) J Neurooncol 118: 109-116; Motzer, R J et al. (2015) N Engl J Med 373: 1803-1813; Furudate, S. et al. (2016) Case Rep Oncol 9: 644-649; Goldberg, S B et al. (2016) Lancet Oncol 17: 976-983; Pai-Scherf, L. et al. (2017) Oncologist 22: 1392-1399]. Checkpoint inhibitors are significantly effective in treating brain metastatic tumors from melanoma and lung cancer, especially when considering the lack of the adaptive immune response in the central nervous system [Id., citing Queirolo, P. et al. (2014) J Neurooncol 118: 109-116; Goldberg, S B et al. (2016) Lancet Oncol 17: 976-983; Di Giacomo, A M et al. (2017) Cytokine Growth Factor Rev 36: 33-38]. Evidence suggests that the effectiveness of checkpoint inhibition in melanoma brain metastasis depends on extracranial disease and peripheral activation of CD8+ T cells [Id., citing Taggart, D. et al. (2018) Proc Natl Acad. Sci 115: E1540-E1549]. On the other hand, a high level of circulating Tregs has been associated with a higher risk of metastasis in non-small lung carcinoma patients. [Id., citing Erfani, N. et al. (2012) Lung Cancer 77: 306-311]. Similar associations have been described in breast cancer [Id., citing Metelli, A. et al. (2016) Cancer Res 76: 7106-7117], colorectal carcinoma metastasis [Id., citing Wang, Q. et al. (2014) Cell Immunol 287: 100-105, and hepatocellular carcinoma [Id., citing Ye, L Y et al. (2016) Cancer Res 76: 818-830].

### B Cells

Upon activation in the germinal centers in lymphoid organs, B cells expressing high-affinity antibodies differentiate into antibody-secreting plasma cells and memory B cells that mediate humoral immunity against pathogens [Id., citing De Silva N S, Klein U. (2015) Nat Rev Immunol 15: 137-148]. Although the presence of B cells in the TME has been described in different carcinomas (including melanoma and breast, ovarian, and prostate cancer, among others) [Id., citing Chin, Y. et al. (1992) Anticancer Res 12: 1463-1466; Yang, C. et al. (2013) PLOS One 8: e54029; Woo, J R et al. (2014) J Transl. Med. 12: 30; Pylayeva-Gupta, Y. et al. (2016) Cancer Discov. 6: 247-255], the role of B cells in cancer progression is much less understood than that of T cells. Accumulating evidence indicates that B cells promote and support tumor growth; for example, using a transgenic mouse model of epithelial carcinogenesis, Coussens and colleagues [Id., citing de Visser, K E et al. (2005) Cancer Cell 7: 411-423] demonstrated that the lack of mature B cells decreases tumor progression. The adoptive transfer of B cells restores chronic inflammation, angiogenesis, and tumor growth. Different mechanisms have been described to explain the protumor role of B cells, from immunosuppression via secretion of IL-10 [Id., citing Schioppa, T. et al. (2011) Proc Natl Acad. Sci 108: 10662-10667] and TGFβ [Id., citing Olkhanud, P B et al. (2011) Cancer Res 71: 3505-3515] to direct stimulation of tumor cell proliferation by B-cell-derived IL-35 in human pancreatic neoplasia and Kras-driven pancreatic neoplasms in mice [Id., citing Pylayeva-Gupta, Y. et al. (2016) Cancer Discov. 6: 247-255]. Also, by deposition of immunoglobulins in the TME, B cells indirectly stimulate angiogenesis and chronic inflammation by activating myeloid cells via FcRγ [Andreu, P. et al. (2010) Cancer Cell 17: 121-134].

Crosstalk Between Different Immune Cells within the TME

There is growing evidence that tumor-associated immune cells act in concert to both control and promote tumor formation. In this sense, during the phase of elimination, NK cells exert a strong tumoricidal role; secretion of CCL5 and XCL1 by NK cells promotes the recruitment of conventional DCs (cDCs) to the TME, resulting in increased priming and activation of new repertoires of anti-tumor T cells, stimulating the overall effector immune response [Id., citing Moretta, A. et al. (2005) Trends Immunol 26: 668-675; Bottcher, J P et al. (2018) NK cells stimulate recruitment of cDC1 into the tumor microenvironment promoting cancer immune control. Cell 172: 1022-1037.e14]. Additionally, the reciprocal interplay between NK cells, effector T cells, and anti-tumor macrophages by the secretion of IFN-γ and TNF-α in the tumor site boosts the differentiation of CTLs, increases macrophage phagocytosis, increases the recruitment of cytotoxic cMET+ neutrophils, and enhances the cytotoxic ability of NK cells [Id., citing Finisguerra, V. et al. (2015) Nature 522: 349-353; Showalter, A. et al. (2017) Cytokine 97: 123-132]. Dectin-1, a pattern recognition receptor (PRR) on macrophages and DCs, recognizes N-glycan structures on tumor cells, which activate the IRF5 pathway responsible for enhancing the killing capacity of NK cells [Id., citing Chiba, S. et al. (2014) eLife 3: e04177]. Moreover, CX3CR-1+ patrolling monocytes inhibit metastatic progression through the recruitment of NK cells to the metastatic site, and then NK cell-derived IFN-γ reprograms macrophages into a tumoricidal effector macrophage state [Id., citing O'Sullivan, T. et al. (2012) J Exp Med 209: 1869-1882].

Once the tumors have escaped from initial tumoricidal immunity, they undergo different strategies that tip the balance toward immune tolerance, with the TAMs and tumor-associated Tregs as key orchestrators of this process, as they dampen the effect of innate and adaptive effector immune cells at various levels and through different mechanisms. For example, TAMs and Tregs boost an immune-tolerant TME by secretion of immune-suppressive molecules such as IL-10, TGF-β, and prostaglandins; they also inhibit the secretion of IL-12 by DCs, avoiding the mounting of a T_{H}1 response and excluding NK and effector T cells [Id., citing Ruffell, B. et al. (2014) Cancer Cell 26: 623-637; Speiser, D E et al. (2016) Nat Rev Immunol 16: 599-611; Frydrychowicz, M et al. (2017) Scand J Immunol 86: 436-443; Mantovani, A. et al. (2017) Nat Rev Clin Oncol 14: 399-416; Tauriello, D V F et al. (2018) Nature 554: 538-543].

### Cancer Heterogeneity and Anti-Tumor Immunosurveillance

Analysis of human primary and metastatic tumors has shown high levels of genomic, phenotypic, and antigenic heterogeneity [Id., citing Swanton C. (2012) Cancer Res 72: 4875-4882], which contribute to therapy failure and disease progression. Various mechanisms have been proposed to explain intratumor heterogeneity: Genomic instability [Id., citing McGranahan N, Swanton C. (2015) Cancer Cell 27: 15-26], hierarchical organization arising from initiating cancer stem cells [Id., citing Kreso A, Dick J E. 2014. Cell Stem Cell 14: 275-291], and selective pressure imposed by the immune system likely impact antigen heterogeneity of the tumor [Id., citing Quail D F, Joyce J A. (2013) Nat Med 19: 1423-1437]. Through cancer immune editing, the immune system eliminates the more immunogenic cancer cells, thus promoting the development of clonal tumors and thereby decreasing the heterogeneity. In contrast, the lack of immune selection likely increases the neoantigen heterogeneity. Analysis of neoantigen heterogeneity in tumor samples from lung cancer and melanoma patients demonstrated that patients with clonal tumors (˜78% of clonality) are more susceptible to T-cell attack and have a more sensitive tumor checkpoint inhibition compared with more heterogeneous tumors (˜8% of clonality) [Id., citing McGranahan, N. et al. (2016) Science 351: 1463-1469]. Moreover, analysis of different areas of heterogeneous tumors showed different levels of antigen-specific CD8+ T cells in different tumor regions [Id., citing McGranahan, N. et al. (2016) Science 351: 1463-1469]. Increases in the mutational burden and heterogeneity of neoantigens in vivo as well as the priming of new anti-tumor T-cell repertoires [Id., citing Rizvi et al. (2015) Science 348: 124 128; Germano, G. et al. Nature (2017) 552: 116-120] result from the inactivation of the DNA repair system in colorectal, breast, and pancreatic cell lines. Interestingly, tumors with high neoantigen burden correlate with good prognosis in lung cancer patients treated with anti-PD1 [Id., citing Rizvi, N A et al. (2015) Science 348: 124-128]. As genomic tumor heterogeneity increases, the probability of subclonal generations escaping immune attack likewise increases.

Metastatic progression and therapy resistance usually proceed from rare clones in primary tumors [Id., citing Gupta G P, Massague J. (2006). Cell 127: 679-695]. Consistent with this view, a deep analysis of intrapatient metastases in a patient with ovarian cancer showed that regressing metastatic tumors were associated with an immune infiltrate characterized by CD4+ and CD8+ T cells and higher tumor mutation and neoepitope load compared with progressing lesions that are associated with T-cell exclusion [Id., citing Jimenez-Sanchez, A. et al. (2017) Cell 170: 927-938.e20].

It is unclear whether the cancer heterogeneity observed in patients is the end result of the immune system's inability to stop tumor progression or whether the mutational burden promotes heterogeneity that leads to immune evasion. Furthermore, the evolving mutation burden, the selective pressure of chemotherapy, and the rapid turnover of inflammatory cells within the primary and metastatic tumor, in conjunction with the nonuniform distribution of immune cells throughout the tumor, likely promote differential selective pressures in disparate tumor regions, allowing for the development of heterogeneous tumors [Id., citing McGranahan, N. et al. (2016) Science 351: 1463-1469].

### T and B Cell Diversity

Antigen receptors with diverse binding activities are the hallmark of T and B cells of the adaptive immune system. These are generated by genomic rearrangement of variable (V), diversity (D) and joining (J) gene segments separated by highly variable junction regions. [Boyd, S C et al. Sci. Trans. Med. (2009) 1 (12): 12ra23, citing Schatz, DG. Semin. Immunol. (2004) 16: 245-56]. The complex repertoire of immune receptors generated by T and B calls enables recognition of diverse threats to the host organism. [Boyd, S C et al. Sci. Trans. Med. (2009) 1 (12): 12ra23]. Expanded clones of B cells with useful antigen specificities persist over time to enable rapid responses to antigens previously detected by the immune system.

T cell receptors (TCRs) are dimeric (αβ or γδ) highly variable T lymphocyte membrane proteins that recognize antigenic peptides presented on heterologous cells by the major histocompatibility complex (MHC) [Freeman, J D, et al. Genome Res. (2009) 19: 1817-24, citing Davis, M M and Bjorkman, PJ Nature (1988) 334: 395-402; Bassing, C H et al Cell (2002) 109 (Suppl): S45-SSS]. Recognition specificity for diverse peptide-MHC (pMHC) complexes is provided by the three complementarity determining regions (CDRs) of the TCR. CDR1 and CDR2) are coded for by germline sequences, while CDR3, the highly polymorphic principal recognition site, is created when TCR genomic loci undergo somatic recombination between gene segments during development of T lymphocytes in the thymus [Id., citing Gellert, M. Annu. Rev. Genet. (1992) 26: 425-446; Gellert, M. Ann. Rev. Biochem. (2002) 71: 101-32; Gellert, 1992, 2002; Jung, D. and Alt, F W. Cell (2004) 116: 299-311]. The CDR3 of each of the B and 8 two receptor chains defines the clonal specificity. For αβ T cells, the CDR3 is in most contact with the peptide bound to the MHC. [Id., citing Rudolph, M G et al. Annu. Rev. Immunol. (2006) 24: 419-66]. For the α locus and the γ locus, recombination occurs between variable (V) and joining (J) segments. For the δ locus and the β locus, there is recombination between V and J segments, but also the inclusion of one of two short diversity (D) segments. At CDR3 recombination junctions, further complexity is generated through the deletion of germline-encoded bases and the addition of random nontemplated bases. The resulting hypervariable sequences of the CDR3 make possible the recognition of diverse peptide-MHC (pMHC) complexes. During T cell maturation, all T cells expressing rearranged receptors capable of binding pMHC with high enough affinity to be biologically relevant are retained (positive selection), but only T cells with rearranged receptors that do not interact strongly with self-pMHC complexes ultimately exit the thymus (negative selection). [Freeman, J D, et al. Genome Res. (2009) 19: 1817-24]. The V(D)J recombination is not entirely random, and the prevalence of specific gene segments and combinations of gene segments shows marked variation in the repertoire. Contributes to this bias are introduced even before thymic selection, through variation in the efficiency of recombination of different gene segments. [Id., citing Manfras, B J et al. Hum. Immunol. (1999) 60: 1090-1100; Krangel, M S. Nat. Immunol. (2003) 4: 624-30].

There are two subsets of T cells based on the exact pair of receptor chains expressed. These are either the alpha (a) and beta (B) chain pair, or the gamma (Y) and delta (8) chain pair, identifying the αβ or γδ T cell subsets, respectively. The expression of the β and δ chain is limited to one chain in each of their respective subsets by allelic exclusion [Yassi, M. B. et al. Immunogenetics (2009) 61: 493-502., citing Bluthman et al 1988; Uematsu et al 1988]. These two chains are also characterized by an additional DNA segment, referred to as the diversity (D) region during the rearrangement process. The D region is flanked by N nucleotides, which constitutes the NDN region of the CDR3 in these two chains. [Id.]

The initial phase of the adaptive immune response involves B and T cell clonal selection based on the structural complementarity of antigen-specific receptors to pathogen-derived epitopes. [Yassi, M. B. et al. Immunogenetics (2009) 61: 493-502, citing Davis, M M and Chien, Y H. In Paul, W E ed. Fundamental Immunology, 5th Ed. (2003) Lippincott Williams & Wilkins, Philadelphia, pp. 227-258; Kolar, G R and Capra, JD In Paul, W E ed. Fundamental Immunology, 5th Ed. (2003) Lippincott Williams & Wilkins, Philadelphia, pp. 47-68]. After pathogen clearance, a proportion of these cells will be retained as memory. Memory provides more rapid and effective immune protection against recurring pathogen present in the environment. The collection of cells that respond to a particular pathogen is referred to as the repertoire. The repertoire recognizing a molecule would be the sum of the repertoires responding against all the component epitopes of the molecule. Likewise, the repertoire against an organism would be the sum of all the repertoires against all the molecules from the pathogen. [Id.]

Wang et al. estimated 0.47×10⁶ TCR-α unique nucleotide sequences and 0.35×10⁶ TCR-β sequences. [Benichou, J. et al., Immunology (2011) 135: 183-191, citing Wang, C., et al. Proc. Natl Acad. Sci. USA (2010) 107: 1518-23]. Robins et al. suggested that CD8+ T cells express <0.1% of the combinatorial landscape of the β chain (5×10¹¹). [Id., citing Robins, H S., et al. Blood (2009) 114: 4099-4107]. These are only lower limits to the actual size of the repertoire, and any individual expresses only a small fraction of the potential diversity. [Id.].

Antibody paratropes are found at the hypervariable region of a light and heavy chain heterodimer. Each chain contributes three loops to a spatial cluster of complementarity determining regions (CDRs). CDR1 1 and 2 are encoded in germline V-segment loci: 51 Vh and 70 Vκ/λ loci, each with unique amino acid encodings exist in a typical human haplotype [Glanville, J. et al., Proc. Natl Acad. Sci USA 106 (48): 20216-20221, citing Huber, C. et al. Eur. J. Immunol. (1993) 23: 2868-75; Kawasaki, K., et al. Genome Res. (1995) 5: 125-135; Matsuda, F. et al. J. Exp. Med. (1998) 188: 2151-2162]. Diversity in each chain is determined by combinatorial VH-(DH)-JH (for the heavy) or Vκ/λ-Jκ/λ (for the light) rearrangements, P and N-addition, junctional flexibility, and somatic hypermutation of variable domain nucleotides, with a concentration on CDR encoding regions [Id., citing Tonegawa, S. Nature (1983) 302: 575-581; Wu, TT, Kabat, EA. J. Exp. Med. 132: 211-250]. The combinatorial association of such stochastically generated light and heavy chains has the potential to generate many orders of magnitude more diversity than can be uniquely displayed on the 10¹¹ B cells in a single individual's lymphocyte population [Id., citing Perelson, A S, Oster, G F. J. Theor. Biol. 81: 645-70; Trepel, F. Klin. Wochenschrift (1974) 52: 511-15]. With each antibody variable fragment (Fv) encoded by at least 650 base pairs, the presented repertoire is potentially 4 orders of magnitude larger than the entire human diploid genome (6.4×10⁹ bp).

The combination of all these sources of diversity generates a vast repertoire of T cell and B cell specificities. The average human immune system can recognize 1012-1015 antigens, meaning that the immune arsenal already stores the means to recognize virtually any foreign molecule. Large numbers of different T-cell and B-cell receptors or clonotypes enable the immune system to protect against many different types of pathogens. As needed, populations of one or more receptor combinations can be expanded to eliminate a new pathogen. With age however, this surveillance system becomes spent and less flexible to mount an immune defense. Any underlying conditions further jeopardize immune preparedness.

A T cell clonotype is a unique nucleotide sequence that arises during the gene rearrangement process for that receptor. The combination of nucleotide sequences for the surface expressed receptor pair would define the T cell clonotype. Clonotyping, the process used to identify the unique nucleotide CDR sequences of a TCR chain, involves PCR amplification of the cDNA using V region-specific primers and either constant region (C) or J region-specific primer pairs, followed by nucleotide sequencing of the amplicon. The diversity index therefore is an expression of the CDR3 clonotype of the T cell β chain out of all the repertoires. It is noted that referring to only one T-cell receptor chain ignores that the actual clonotype of a T-cell consists of the combination of both alpha and beta receptor chains. A further complication is when homodimers form receptors, e.g., alpha: alpha and beta: beta. These considerations would actually increase the potential diversity.

### Tumor Cell Evasion of the Immune Response

Tumor cells evade the immune attack using two main strategies: avoiding their immune recognition and instigating an immunosuppressive TME.

In the first, cancer cells may lose the expression of tumor antigens on the cell surface, thus avoiding recognition by cytotoxic T cells. For example, 40% of non-small cell lung cancers hold a loss of heterozygosity in human leukocyte antigens (HLAs), which leads to immune escape by presenting fewer antigens [Id., citing McGranahan, N. et al. (2017). Cell 171: 1259-1271 e1211]. HLA loss has been associated with resistance to T-cell transfer therapy in metastatic colorectal cancer [Id., citing Tran, E. et al. (2016) N Engl J Med 375: 2255-2262] and poor outcome response to checkpoint blockade immunotherapy in melanoma and lung cancer patients [Id., citing Chowell, D. et al. (2018) Science 359: 582-587]. Thus, mutations and deletions may result in down-regulation of the antigen-presenting machinery and likely confer resistance to T-cell effector molecules, such as TNF-α and IFN-γ [Id., citing Patel, S J et al. (2017) Nature 548: 537-542]. Additionally, to overcome the attack of NK cells in experimental metastasis, breast and lung cancer cells down-regulate cell surface NK activators, becoming invisible to detection by NK cells [Id., citing Malladi, S. et al. (2016) Cell 165: 45-60].

In the second, cancer cell-derived factors instigate an immune-tolerant TME by (1) secretion of suppressive molecules such as IL-10, TGF-β, prostaglandin E2, and VEGF [Id., citing Gabrilovich, D I et al. (1996) Nat Med 2: 1096-1103; Massague, J. (2008) Cell 134: 215-230; Dominguez-Soto, A. et al. (2011) J Immunol 186: 2192-2200; Bottcher, J P et al. (2018) Cell 172: 1022-1037.e14]; (2) expression of inhibitory checkpoint molecules such as PD-L1, CTLA-4, and V domain immunoglobulin suppressor of T-cell activation (VISTA) [Id., citing Topalian S L et al. (2012) Cancer Cell 27: 450-461; Snyder, A. et al. (2014) N Engl J Med 371: 2189-2199; Boger, C. et al. (2017) Oncoimmunology 6: e1293215]; and (3) induction of the recruitment of TAMs, MDSCs, and Tregs by tumor-derived chemokines such as CCL2, CSF1, CCL5, CCL22, CXCL5, CXCL8, and CXCL12 [Id., citing Weitzenfeld, P. and Ben-Baruch, A. (2014) Cancer Lett 352: 36-53; Kumar, V. et al. (2016) Trends Immunol 37: 208-220; Mantovani, A. et al. (2017) Nat Rev Clin Oncol 14: 399-416; Tanaka A, Sakaguchi S. (2017) Cell Res 27: 109-118]. Combined, these strategies result in a complex and efficient system for immune evasion.

### Chemokines in the Cancer Microenvironment

Chemokine receptors CCR4, CCR5, CXCR3, CXCR4, CCR6, and CCR7 play a pivotal role in the regulation of T cell homing to inflammatory sites [Vilgelm, A E and Richmond, A. Front. Immunol. (2019) doi.org/10.3389/fimmu.2019.00333, citing Hamann, A., Syrbe, U. Rheumatolog. (2000) 39: 696-913]. T cells (αβ, γδ, T_{FM}, T_{FH}, T_{H}22, Tregs, ILCs, NKT), NK cells, B cells and immature DCs [Id., citing Hartawicg, T. et al. Eur. J. Immunol. (2015) 45: 3022-3033; Ramirez-Valle, F. et al., Proc. Nat'l. Acad. Sci. USA (2015) 112: 8046-8051; Zhang, Y. et al. Elife (2016) 5: e18156] are recruited to a tumor by CCL20 interaction with CCR6. CCL19 and CCL21 recruit Tregs, CD4T helper, TCM, TRCM, activated T cells, monocyte-derived dendritic cells (mDC) and B cells to the TME through interaction with CCR7 [Id., citing Damas, J K, et al. Clin. Exp. Immunol. (2009) 157: 400-407; Henning, G. et al. J. Exp. Med. (2001) 194: 1875-1881; Ato, M. et al. Nat. Immunol. (2002) 3: 1185-1191; Kozai, M. et al. J. Exp. Med. (2017) 214: 1925-1935; Ueno, T. et al. Immunity (2002) 16: 205-218]. Dendritic cells home to XCR1, CCL3, CCL4, CCL5, CCL20, and CCL25 in the TME or lymph node (LN) [Id., citing Corrales, L. et al. Cell Res. (2017) 27: 960108; Gajewski, T F et al. J. Clin. Oncol. (2009) 27: 9002; Ayers, M. et al. J. Clin. Invest. (2017) 127: 2930-2940]. When antigen-specific CD4 T cells interact with DC, CCL3, and CCL4 are released and this can guide CCR5-positive naïve CD8+ T cells into tissues for activation [Id., citing Catellino, F. et al. Nature (2006) 440: 890-895]. As such, secretion of ligands for these receptors (CCL4/5 for CCR5, and CXCL9/10/11 for CXCR3) at the site of inflammation is necessary for the initiation of a specific immune response [Id., citing Oelrug, C., Ramage, J M. Clin. Exp. Immunol. (2014) 178: 1-8].

Tumor-promoting leukocytes are comprised of macrophages expressing arginase, IL4, IL10, and IL13, as well as myeloid-derived suppressor cells (MDSCs), T regulatory cells (Tregs) and specific B cell subsets. Ligands for chemokine receptors CCR1, CCR2, CCR3, and CCR5, CCR8, CXCR1, CXCR2, and CXCR4 recruit macrophages to the TME [Id., citing Nagarsheth, N. et al. Nat. Rev. Immunol. (2017) 17: 559-724, Kitamura, T. et al. J. Exp. Med. (2015) 212: 1043-1059; Saji, H. et al. Cancer (2001) 92: 1085-1091; Azenshtein, E. et al. Cancer Res. (2002) 62: 1093-1102; Highfill, S L et al. Sci. Transl. Med. (2014) 6: 237ra67; Kumar, S. et al. J. Clin. Invest. (2018) 128: 5095-5109; Sawanobori, Y. et al. Blood (2008) 111: 5457-5466; Taki, M. et al. Nat. Commun. (2018) 9: 1685; Wang, D. et al. Cancer Res. (2017) 77: 3655-3665; Mauldin, I S et al. Cancer Immunol. Immunother. (2016) 65: 1189-1199; Zhang, H. et al. Front. Immunol. (2017) 8: 129; Zhu, H. et al. Oncotarget (2017) 8: 114554-114567; Yang, L. et al. Cancer Cell (2008) 13: 23-35; Ba, H. et al. Int. Immunopharmacol. (2017) 44: 143-152; Obermajer, N. et al. Cancer Res. (2011) 71: 463-470]. Neutrophils and myeloid derived suppressor cells (MDSCs) are recruited to the tumor through ligands for CCR2, CCR3, CXCR1, CXCR2, and CXCR4. Tregs express the chemokine receptors CCR2, 3, 4, 6, 7, 8, 10, CXCR3, and CXCR4 [Id., citing Durr, C. et al. Cancer Res. (2010) 70: 10170-81; Erhardt, A. et al. J. Immunol. (2011) 186: 5284-5293; Lli, C X et al. J. Hepatol. (201) 65: 944-952; Lunardi, S. et al. Oncotarget (2014) 5: 11064-11080; Paust, H J, et al. J. Am. Soc. Nephrol. (2016) 27: 1933-1942; Righi, E. et al. Cancer Res. (2011) 71: 5522-5534; Zou, L. et al. Cancer Res. (2004) 64: 8451-8455; Zhao, E. et al. Oncoimmunology (2012) 1: 152-161; Facciabene, A. et al. Nature (2011) 475: 226-230]. Because the same chemokines that recruit anti-tumor leukocytes can also recruit pro-tumor leukocytes (for example CCL19 and CCL21 recruit both Tregs, mDCs, and activated T cells), therapeutically targeting chemokines or chemokine receptors in cancer is complicated.

For naïve T cells to become activated, antigen presenting DCs migrate from the developing tumor to the lymph node where they present antigen to the T cells via the T cell receptor (TCR) and stimulate a process that leads to T cell activation. CD4 cells can be activated by antigen presenting cells (APCs) and mature into helper cells [T helper type I cells (Th1) or T helper type II cells (Th2)]. Th1 cells produce cytokines including interferon-γ (IFNγ), tumor necrosis factor-alpha (TNFα), while Th2 cells secrete IL-4, IL-5, IL-10, and IL-13. The cytokines produced by the DCs influence the differentiation of naïve helper T cells into either Th1 or Th2 cells. For example, if DCs secrete IL-12, the naïve helper T cells differentiate into Th1 cells. Th1 cells express CD40L on their plasma membrane and this ligand binds to CD40 expressed by the DC or other APC. Engagement of CD40 on the DCs or other APC primes them to a higher activation level resulting in elevated expression of class I MHC, B7 and co-stimulating molecules such as 4-1BBL. When CD8+ T cells come into contact with one of these highly activated DCs, its TCRs recognizes the peptides presented by the MHC Class I molecule on the DC/APC. This, in turn, leads to the activation of a CD8 T cell upon binding of its TCR to the MHC presented peptide [Id., citing Gregor, C E et al. Adv. Immunol. (2017) 135: 119-121]. The clone subsequently expands in response to IL-2 induced stimulation of cell proliferation. CD4 T cells are important for the survival and expansion of activated CD8 T cell clones and for the survival of memory CD8 T cells during recall expansion, but there is some priming in the absence of Major Histocompatibility Complex, Class II (MHCII) activation [Id., citing Bao, X. et al. Immunity (2010) 33: 817-829].

Different subsets of T cells migrate in response to a variety of chemokines [Id., citing Gregor, C E et al. Adv. Immunol. (2017) 135: 119-121]. For example, CCR7 is expressed on all naïve CD4 T cells and its ligand CCL21 is expressed by the endothelial cells of the high endothelial venules (HEV) which are specialized vessels that facilitate lymphocyte recruitment. CCL21 is presented by heparin sulfate into the luminal surface [Id., citing Bao, X. et al. Immunity (2010) 33: 817-29]. CCL19 can also bind to CCR7 on CD4 cells and is thought to mediate survival of naïve T cells as they move into the LN [Id., citing Link, A. et al. Nat. Immunol. (2007) 8: 1255-65]. Once in the LN, naïve CD4 T cells search for APCs using a random walk along a fibroblastic reticular cell network [Id., citing Bajenoff, M. et al. Immunity (2006) 25: 989-1001], which expresses adhesion molecules in addition to ligands for CCR7, CCL19, and 21, as well as CXCL12, which binds CXCR4. To escape the LN, CCR7 gradually becomes down-regulated and the CD4 cells bind the sphingosine-1-phosphate receptor 1 (S1PR1) [Id., citing Pham, T H et al. Immunity (2008) 28: 122-33] and follow S1P signals into the lymphatic vesicles, other LNs, or the circulation. FOXO1 is a key transcription factor in CD4 T cells, as is KLF2. FOXO1 regulates the expression of CD62L and CCR7, while KLF2 represses CXCR3, CCR3 and CXCR5 expression [Id., citing Sebzda, E. et al. at. Immunol. (2008) 9: 292-300].

When CD4 T cells are activated, there is upregulation of CXCR3 and CXCR5, both of which are associated with differentiation into T_{H}1 cells [Id., citing Woodruff, M C et al. J. Exp. Med. (2014) 211: 1611-1621] and can be linked to Bcl6 and cell division, though the order is controversial (55, 56). TCR engagement, IL12, IL21, and IFNγ expression along with induction of T-bet are associated with escape from a plastic state into a definitive T_{H}1 phenotype [Id., citing Nakayamada, S. et al. Immunity (2011) 35: 919-931]. The cells migrate from the T zone to the B-T zone interface using CXCR5 and EB12 [Id., citing Li, J. et al. Nature (2016) 533: 110-114] to escape areas with high IL-2. In contrast, contact with an environment high in IL-2 will suppress T_{FH} differentiation.

CD4+ T cells undergo priming by DCs and upregulate CXCR3 expression, then CXCR3 mediates the migration of CD4+ T cells between different DC populations in the LN. These CD8a+ DCs are producing CXCL10 in response to IFNγ stimulation. CXCL9, CXCL10, and CXCL11 are produced by many cell types including fibroblasts, leukocytes, and keratinocytes and all bind CXCR3, although the most potent ligand in humans for CXCR3 is CXCL11 [Id., citing Van Raemdonck, K. et al. Cytokine Growth Factor Rev. (2015) 26: 311-27]. CXCR3 is essential for T cell recruitment into tumors and through the thymus [Id., citing Luster, AD, Leder, P. J. Exp. Med. (1993) 178: 1057-65; Mikucki, M E et al. Nat. Commun. (2015) 6: 7458] and Th1 cells also produce IFNγ that induces additional production of CXCL9 and 10 to enhance the recruitment of cytotoxic CD8+ T cells into the tumor [Id., citing Nakanishi, Y. et al. Nature (2009) 462: 510-513].

In the tumor microenvironment, chemokines are produced by tumor cells, endothelial cells, mesenchymal stem cells (MSC), cancer-associated fibroblasts, myeloid cells, and neutrophils, providing a very rich “soil” to facilitate the recruitment of immune cells into the tumor microenvironment (TME). For example, tumor cells, macrophages, and neutrophils produce CXCL1, CXCL2, CXCL5, and CXCL8 and these chemokines recruit MDSCs, both the PMN-MDSCs and the Monocytic-MDSCs [Id., citing Kumar, V. et al. Cancer Cell (2017) 32: 654-68; Gabrilovich, DI. Cancer Immunol. Res. (2017) 5: 3-8]. The MDSCs suppress the activity of CD8+ T effector cells to prevent tumor cell killing by these cells. Dendritic cells (DCs), Tregs, CD8+ T cells, Th1, Th9, Th17, TEM, TRM, and macrophages are recruited into the TME by CCL3-5, CCL8, CCL11-12, and CCL28 [Tiberio, L. et al. Cell Mol. Immunol. (2018) 15: 346-352]. Mature DCs release CXCL5, CXCL9-11 and these chemokines recruit CD4+ Th cells, CD8+ T cells, Tregs, pDCs, NK, and NKT cells into the TME [Id., citing Sokol, CL, Luster, AD. Cold Spring Harb. Perspect. Biol. (2015) 7: a016303].

### Hepatocellular Carcinoma

Hepatocellular carcinoma (HCC), the leading type of primary liver cancer and a significant global health burden, is a solid tumor with a high degree of capillarization and arterialization. [Yao, C., et al. Cancer Giol. Med. (2023) 20 (1): 25-43]. HCC ranks as the third leading cause of cancer-related deaths worldwide, with its incidence and mortality rates on the rise [Argentiero, A. et al. J. Clinical Med. (2023) 12: 7469, citing Fitzmaurice, C., et al. JAMA Oncol. (2017) 3: 1683-1691]. The increasing prevalence of HCC can be attributed to various factors, including the growing prevalence of chronic liver diseases, such as cirrhosis, hepatitis B and hepatitis C infections, and nonalcoholic fatty liver disease (NAFLD) [Id., citing Forner, A., et al. Lancet (2018) 391: 1301-1314].

### Tumor Microenvironment (TME)

The tumor microenvironment (TME) in HCC consists of a complex network of cellular and non-cellular components that interact dynamically to shape the behavior and progression of tumors that play a critical role in tumor growth, invasion, metastasis, and therapeutic resistance.

### Cellular Components.

Cancer-Associated Fibroblasts (CAFs.) Cancer-associated fibroblasts (CAFs), which are activated fibroblasts that have acquired distinct characteristics and functions in response to signals from cancer cells and the TME, are the most abundant cell type in the HCC tumor TME and play a crucial role in tumor progression and metastasis. CAFs. They secrete various factors, including growth factors, cytokines, and extracellular matrix (ECM) proteins, which promote tumor cell proliferation, angiogenesis, immune suppression, and therapeutic resistance in HCC [Id., citing Kalluri, R. Nat. Rev. Cancer (2016) 16: 582-598; Mueller, S N and Germain, RN. Nat. Rev. Immunol. (2009) 9: 618-629; Kubo, N., et al. World J. Gastroenterol. (2016) 22: 6841-6850]. CAFs contribute to the remodeling of the ECM, creating a supportive niche for tumor growth and invasion [Id., citing Kallluri, R. Nat. Rev. Cancer (2016) 582-598]. The extracellular matrix (ECM), which is mainly secreted by cancer-associated fibroblasts (CAFs), which produce more ECM proteins than normal fibroblasts, is composed of various macromolecules, including collagens, glycoproteins (fibronectin and laminins), proteoglycans and polysaccharides with different physical and biological properties. [Brassart-Pasco, S., et al. Front. Oncology (2020) 10: 397]. Interstitial matrix, primarily synthesized by stromal cells, is rich in fibrillary collagens and proteoglycans. CAF secretome analyses show an increased secretion of bone morphogenetic protein (BMP)-1, thrombospondin-1 and elastin interface 2 [Id., citing Santi, A., et al. Proteomics (2018) 18: e1700167; Socovich, A M and Naba, A. Semin. Cell Dev. Biol. (2019) 89: 157-166].

CAFs interact with other cell types within the TME, such as immune cells and endothelial cells, through paracrine signaling and direct cell-cell contact, further facilitating tumor progression and metastasis [Argentiero, A., et al. J. Clinical Med. (2023) 12: 7469, citing Kalluri, R. Nat. Rev. Cancer (2016) 16: 582-598; Mueller, S N and Germain, RN. Nat. Rev. Immunol. (2009) 9: 618-629]. They also play a role in drug resistance: CAF-derived and secreted phosphoprotein 1 (SPP1) enhances tyrosine-kinase inhibitor resistance by activating alternative oncogenic signals and promoting epithelial-to-mesenchymal transition.

Immune cells. The immune response within the HCC TME is dysregulated, leading to immune evasion and tumor progression. Various immune cell populations have been identified in the HCC TME, including tumor-associated macrophages (TAMs), myeloid-derived suppressor cells (MDSCs), and regulatory T-cells (Tregs).

Tumor-Associated Macrophages (TAMs). TAMs are key regulators of the immune response in HCC. They exhibit a distinct polarization toward an M2-like phenotype, characterized by the secretion of anti-inflammatory cytokines and growth factors, such as interleukin-10 (IL-10) and transforming growth factor-beta (TGF-β), promoting angiogenesis, tissue remodeling, and immune suppression [Id., citing Zhang, Q., et al. Cell (2019) 179: 829-845]. TAMs also inhibit T-cell activation and function through the secretion of inhibitory molecules, including programmed death-ligand 1 (PD-L1), thereby contributing to immune evasion in HCC [Id., citing Zheng, H., et al. (2023) 9: 65].

Myeloid-derived suppressor cells (MDSCs). MDSCs, a heterogeneous population of immature myeloid cells with immunosuppressive properties, contribute to immune suppression in HCC. MDSCs inhibit T-cell responses through various mechanisms, such as the production of arginase-1 and inducible nitric oxide synthase (iNOS), leading to the depletion of essential nutrients and the generation of reactive oxygen species (ROS) [Id., citing Fu, J., et al. Gastroenterology (2007) 132: 2328-2339]. This inhibitory environment hampers effective anti-tumor immune responses and promotes tumor progression.

Regulatory T cells (Tregs). Tregs are a specialized subset of CD4+ T cells that play a critical role in maintaining immune homeostasis and in preventing excessive immune responses. In the HCC TME, Tregs accumulate and exert their suppressive effects by inhibiting effector T-cell responses and promoting tolerance to tumor antigens [Id., citing Fu, J., et al. Gastroenterology (2007) 132: 2328-2339]. The presence of Tregs in the TME has been associated with poor prognosis and reduced survival in HCC patients.

Non-parenchymal liver cells. Liver is an immune organ with several immunocompetent cells. Non-parenchymal resident cells, such as Kupffer cells, hepatic stellate cells (HSC), and liver sinusoidal endothelial cells (LSEC), cooperate in the maintenance of immune tolerance.

Kupffer cells are liver-resident macrophages that act as antigen-presenting cells (APC) to form the first line of defense against pathogens [Chen, C., et al. Front. Immunology (2023) 14: 1133308, citing Ebrahimimkhani, M R, et al. Hepatol. (Baltimore, MD) (2011) 54 (4): 1379-1387; Keenan, B P, et al. J. Immnotherapy Cancer (2019) 7 (1): 267]. They can contribute to hepatocarcinogenesis and immune escape by several mechanisms: 1) secretion of immunosuppressive cytokines (e.g., IL-10) [Id., citing Knolle, P. et al. J. Hepatol. (1995) 22 (2): 226-229]; 2) upregulation of inhibitory immune checkpoint ligand PD-1 [Id., citing Heymann, F. et al. Hepatol. (Baltimore MD) (2015) 62 (1): 279-291]; 3) downregulation of costimulatory molecules (CD80 and CD86) [Id., citing Ringelban, M., et al. Nat. Immunol. (2018) 19 (3): 222-232; Hou, J., et al. J. Hepatol. (2020) 72 (1): 167-182]; 4) production of Indoleamine 2-3 dioxygenase (IDO) [Id., citing Yan, M L, et al. World J. Gastroenterol. (2010) 16 (5): 636-640; and 5) recruitment of Treg cells and of T helper 17 (TH17) cells [Id., citing Ringelban, M., et al. Nat. Immunol. (2018) 19 (30): 222-232; Heymann, F., et al. Hepatol. (1995) 22 (20): 226-229; Hou, J., et al. J. Hepatol. (2020) 72 (1): 167-182]. The interaction of PD-L1 expressed by Kupffer cells and PD-1 expressed by T cells leads to T-cell exhaustion in human HCC [Id., citing Wu, K., et al. Cancer Res. (2009) 69 (20): 8067-8075].

HSCs can secrete hepatocyte growth factor (HGF) that enables MDSC and Treg cells to accumulate inside the liver [Id., citing Hochst, B., et al. J. Hepatol. (2013) 59 (30): 528-535]. Also, HSCs express high levels of PD-L1 to induce T cell apoptosis [Id., citing Dunham, R M, et al. J. Immunol. (Baltimore MD 1950) (2013) 190 (5): 2009-2016]. HSCs can transdifferentiate into CAFs and consequently promote angiogenesis. [Yao, C., et al. Cancer Biol. Med. (2023) 20 (1): 25-43].

LSECs, which line the low shear, sinusoidal capillary channels of the liver and are the most abundant non-parenchymal hepatic cell population, have a critical role in maintaining immune homeostasis within the liver and in mediating the immune response during acute and chronic liver injury. LSECs have potent scavenger capabilities by virtue of their expression of many scavenger receptors, including mannose receptor (MR), CD32, stabilin 1, stabilin 2, Scavenger Receptor B1 (SRB1) and Scavenger Receptor Class F member 1 (SCARF 1), Liver/lymph node-Specific ICAM3-grabbing Non-integrin (LSIGN), Lymphatic Vessel Endothelial hyaluronic acid receptor 1 (LYVE1) and pro-LDL Receptor-related Protein 1 (LRP1). Scavenger receptors are a diverse family of pattern recognition receptors that, like TLRs, are highly evolutionarily conserved. The high levels of scavenger receptors on LSECs give them a high endocytic capacity. LSECs constitutively express low levels of intercellular adhesion molecule 1 (ICAM1), ICAM2 and vascular cell adhesion protein 1 (VCAM1).

Minimal chemokine expression is seen in unstimulated LSECs, although they will express factors such as CXC-chemokine ligand 9 (CXCL9)-CXCL11, CC-chemokine ligand 25 (CCL25), CX3C-chemokine ligand 1 (CX3CL1) and CXCL16 in response to cytokine stimulation. They can also present chemokines derived from neighboring or underlying cells to promote binding and migration of immune cell subsets. In addition to their roles in pathogen recognition and as antigen-presenting cells, LSECs also have a critical role in regulating the recruitment of leukocytes into liver tissue. LSECs play a role in the quiescence of HSCs, which is lost during capillarization of LSECs, which permits HSC activation and fibrogenesis. [Id., citing Shetty, S., et al. Nat. Rev. Gastroenterol. Hepatol. (2018) 15 (9): 555-567].

During cirrhosis and chronic hepatitis, LSECs can undergo capillarization, which is mechanistically linked to the development of chronic inflammatory disease. [Shetty, S., et al. Nat. Rev. Gastroenterol. Hepatol. (2018) 15 (9): 555-567, citing Couvelard, A., et al. Am. J. Pathol. (1993) 143: 738-752]. In rodent models, capillarization is associated with enhanced antigen presentation and cytotoxic T cell priming during fibrosis [Id., citing Connolly, M K, et al. J. Immunol. (2010) 185: 2200-2208], and in nonalcoholic steatohepatitis (NASH), capillarization precedes and contributes to the transition from simple steatosis to steatohepatitis [Id., citing Miyao, M., et al. Lab Invest. (2015) 95: 1130-1144].

The changes that occur in LSECs in response to chronic inflammation also affect angiogenic pathways. Neo-angiogenesis is a key feature of chronic liver disease; the majority of neo-vessels arise from portal vein branches and are closely associated with areas of fibrogenesis [Id., citing Onori, P., et al. J. Hepatol. (2000) 33: 555-563; Fernandez, M., et al. J. Hepatol. (2009) 50: 604-620]. A key initiating step is the capillarization of LSECs, which leads to increased hepatocyte hypoxia and subsequent release of pro-angiogenic factors [Id., citing Corpechot, C., et al. Hepatology (2002) 35: 1010-1021; Rosmorduc, O., et al. Am. J. Pathol. (1999) 155: 1065-1073]. The LSEC response is context-specific; for example, acute injury can induce CXCR7 expression and a regenerative response, whereas chronic injury leads to CXCR4 induction, HSC proliferation and fibrogenesis [Id., citing Ding, B S, et al. Nature (2014) 505: 97-102]. During ischemia-reperfusion injury, LSECs develop a pro-inflammatory, prothrombotic phenotype associated with vasoconstriction [Id., citing Peralta, C., et al. J. Hepatol. (2013) 59: 1094-1106]. These changes have been directly linked to neutrophils because IL-33 released by LSECs during ischemia-reperfusion injury triggers the release of neutrophil extracellular traps (NETs), which exacerbate acute hepatic injury [Id., citing Yazdani, H O, et al. J. Hepatol. (2017) 678: 130-39]. In chronic injury, the changes in endothelial phenotype that accompany capillarization and precede fibrosis have been linked to alterations in signaling via the Hedgehog gene family [Id., citing Xie, G., et al. Gut (2013) 62: 299-309] and lead to vasoconstriction and increased intrahepatic vascular resistance due to reduced nitric oxide production by LSECs [Id., citing Rockey, D C and Chung J J. Gastroenterology (1998) 114: 344-351]. Tumor progression in hepatocellular carcinoma is associated with changes in the phenotype of peritumoral LSECs and increased production of angiogenic factors including IL-6 [Id., citing Zhang, P Y et al. BMC Cancer (2015) 15: 830; Geraud, C., et al. Liver Int. (2013) 33: 1428-1440].

CAFs can trigger NK cell dysfunction by secreting prostaglandin E2 (PGE2) and IDO, and prompt MDSC production by releasing IL-16 and CXCL12 [Id., citing Deng, Y., et al. Oncogene (2017) 36 (8): 1090-101].

### Non-Cellular Components

Extracellular Matrix (ECM). The ECM is a complex network of proteins and polysaccharides that provides structural and biochemical support to cells within the TME. In HCC, the ECM undergoes dynamic changes that promote tumor growth, invasion, and metastasis. Alterations in the composition of ECM, remodeling enzymes, and stiffness affect cellular behaviors, such as cell adhesion, migration, and signaling pathways that are involved in tumor progression [Id., citing Winkler, J., et al. Nat. Commun. (2020) 11: 5120]. The dysregulated ECM in HCC contributes to the invasive and metastatic behavior of tumor cells by providing a physical scaffolding and modulating cellular signaling events. Additionally, the abnormal ECM can create a barrier that limits the penetration and efficacy of therapeutic agents.

Hypoxia and Angiogenesis. Angiogenesis in HCC is robustly stimulated by hypoxia. It arises due to the rapid proliferation of tumor cells, insufficient vascularization, and the abnormal architecture of tumor blood vessels. Hypoxia develops within the solid tumors, because of the high interstitial pressure and the distance between the tumor cells and adjacent capillaries, Pro-angiogenic factors (e.g., vascular endothelial growth factors (VEGFs), platelet derived growth factors (PDGFs), fibroblast growth factors (FGFs) and angiopoietins) stimulate the proliferation and migration of ECs from the vessels in the surrounding tissues. Several cytokines also play a role in tumor angiogenesis. [Yao, C., et al. Cancer Biol. Med. (2023) 20 (1): 25-43].

Hypoxia as a hallmark of the TME presents in the majority of tumors and arises from an imbalance between increased oxygen consumption and inadequate oxygen supply. Although the rapid proliferation of tumors can stimulate the growth of new vasculature and tumor-induced angiogenesis leads to the unorganized growth of vasculature, the precisely distributed vasculature in normal tissues contributes to the delivery of oxygenated blood. In contrast, the irregular distribution of tumor vasculature caused by persistent hypoxic conditions can result in an increase in the distance between the capillaries, exceeding the capacity of oxygen to diffuse [Jing, X., et al. Molecular Cancer (2019) 18: 157, citing Wigerup, C. et al. Pharmacol. Ther. (2016) 164: 152-169; Wilson, W R and Hay, MP. Nat. Rev. Cancer (2011) 11: 393-410]. Such chronic hypoxia or diffusion-restricted hypoxia causes the necrosis of tumor cells within the 180-μm periphery of blood vessels. Current anticancer strategies target only tumor cells around the blood vessels rather than those in poorly perfused regions [Id., citing Loeges, S., et al. Cancer Cell (2009) 15: 167-170; Minchinton, A I and Tannock, IF. Nat. Rev. Cancer (2006) 6: 583-592].

Hypoxia induces changes in gene expression and subsequent proteomic changes that have many important effects on various cellular and physiological functions, ultimately limiting patient prognosis [Jing, X, et al. Molecular Cancer (2019) 18: 157, citing Roma-Rodrigues, C., et al. Int'l J. Mol. Sci. (2019) 20]. For example, slowly dividing cells in hypoxic regions can escape most of the cytotoxic drugs that target rapidly dividing cells, and cancer stem cells may also be present in poorly hypoxic regions ensuring epithelial-to-mesenchymal transition (EMT) [Birner, P. et al. Cancer Res. (2000) 60: 4693-4696]. Hypoxia also generates intratumoral oxygen gradients, contributing to the plasticity and heterogeneity of tumors and promoting a more aggressive and metastatic phenotype.

Under hypoxic conditions, hypoxia-inducible factors (HIFs), particularly HIF-1α and HIF-2α, are stabilized and translocated to the nucleus, where they activate the expression of genes involved in angiogenesis, glycolysis, and cell survival [Argentiero, A., et al. J. Clinical Med. (2023) 12: 7469, citing Guo, Y. et al. Oncol. Rep. (2020) 43: 3-15]. In HCC, hypoxia-induced HIF activation promotes the secretion of pro-angiogenic factors, including vascular endothelial growth factor (VEGF), platelet-derived growth factor (PDGF), and angiopoietin-2 (Ang-2), which stimulate the formation of new blood vessels and the recruitment of endothelial cells [Villa, E., et al. Gut (2016) 65: 861-869]. This hypoxia-driven angiogenic response supports tumor growth, provides nutrients and oxygen to tumor cells, and facilitates metastasis by promoting the formation of abnormal and leaky blood vessels.

Hypoxia causes vascular leakage and abnormal lymphatic drainage in the tumor, leading to an increase in interstitial fluid pressure [Jing, citing Nelson, D A, et al. Genes Dev. (2004) 18: 2095-2107]. To adapt to low levels of oxygen and nutrients, tumor cells develop new blood vessels by de novo angiogenesis. However, such newly formed blood vessels are leaky because of their discontinuous endothelium, and, along with the obstruction of lymphatic drainage, produces vascular hyperpermeability and enhanced permeation [Id., citing Maeda, H., et al. J. Control Release (2000) 65: 271-284].

### Hypoxia-Inducing Factor

Hypoxia-inducible factor (HIF) is a heterodimer composed of two basic helix-loop-helix proteins of the Per-ARNT-Sim (PAS) family: an oxygen-sensitive α-subunit and a constitutively expressed β-subunit [Id., citing Semenza, GL. Nat. Rev. Cancer (2003) 3: 721-732]. Three HIF-α isoforms have been identified in mammals. When compared with HIF-1, a transcriptional nucleoprotein with a wide range of target genes, HIF-2 seems to be more restricted in expression in the tissue and less is known about HIF-3 [Id., citing Wiesener, M S et al. FASEB J. (2003) 17: 271-273]. HIFs play a distinct role in tumorigenesis, and immunohistochemical analyses show that HIF-1α and HIF-2 α are overexpressed in most human cancers.

Under normoxic conditions, two critical proline residues in HIF-α subunits are subject to hydroxylation within their oxygen-dependent degradation domain by enzymes called HIF prolyl hydroxylase domain family proteins (PHDs), which use oxygen, ferrous iron, and α-ketoglutarate as substrates. PHDs are HIF-preserved hydroxylases found in mammals, with three subtypes, PHD1, PHD2, and PHD3, as regulators of HIF-1α oxygen sensors to participate in the degradation of HIF-1a. PHD2 keeps HIF-1α at a stable low level in an anoxic environment as the main rate-limiting enzyme, and its activity is controlled mainly by the intracellular oxygen concentration. Then, the von Hippel-Lindau tumor suppressor protein (pVHL) interacts with HIF-α as a result of hydroxylation and recruits an E3 ubiquitin ligase complex, resulting in ubiquitination and subsequent proteasomal degradation of HIF-α.

Hypoxia induces several complex intracellular signaling pathways, such as the major HIF pathway, the PI3K/AKT/mTOR pathway [Muz, B., et al. Hypoxia (Auk) (2015) 3: 83-92, citing Agani, F. and Jiang, B H. Curr. Cancer Drug Targets (2013) 13 (3): 245-251; Courtnay, R, et al. Mol. Biol. Rep. (2015) 42 (4): 841-851], the MAPK/ERK pathways [Id., citing Seta, K A, et al., Sci STKE (2002) 2002 (146): re11; Sanchez, A., et al. J. Alzheimer's Dis. (2012) 32 (3): 587-597; Minet, E., et al. FEBS Lett. (2000) 468 (1): 53-58] and NFκB signaling pathways [Id., citing Koong, A C, et al. Cancer Res. (1994) 54 (6): 1425-1430]. These pathways are involved in cell proliferation, survival, apoptosis, metabolism, migration and inflammation.

Under hypoxic conditions, HIF-1α mediates hypoxia-induced signaling, which plays a role in multiple steps of the transfer cascade [Jing, citing Semenza, GL. Annu. Rev. Pathol. (2014) 9: 47-71]. The inhibitory hydroxylation of HIF-α is reduced, leading to the stability and translocation of HIF-α to the nucleus, where it heterodimerizes with HIF-β [Id., citing Semenza, GL. Oncogene (2010) 29: 625-634]. The HIF-α/β dimer binds with the transcriptional coactivator p300/CBP and hypoxia response element to induce the expression of the HIF target gene located in the promoter region [Id., citing Majmundar, A J, et al. Mol. Cell (2010) 40: 294-309; Semenza, GL. Annu Rev. Pathol. (2014) 9: 47-71]. The development of an abnormal vasculature and a hypoxic microenvironment promotes abnormal angiogenesis, desmoplasia (meaning the formation of fibrous connective tissue by proliferation of fibroblasts), and inflammation, all of which contribute to tumor progression and therapeutic resistance [Id., citing Jain, R K. Cancer Cell (2014) 26: 605-622; Whatcott, C J et al. Cancer J. (2015) 21: 299-306].

In a hypoxic environment, activated HIF-1α increases the activity of Snail and Twist, two transcription factors that reduce E-cadherin expression and promote EMT. While EMT-related signaling is not required for the metastatic process, it promotes invasion, aging, cancer stem cell-like phenotype, and resistance to chemotherapy [Id., citing Thiery, J P, et al. Cell (2009) 139: 871-890]. HIF-1α can also intervene in the expression of enzymes that polymerize and regulate the alignment of collagen fibers and activity of integrins to promote cancer migration [Id., citing Semenza, GL. Annu Rev. Pathol. (2014) 9: 47-71]. Leaky and compressed blood and lymphatic vessels mediated by HIFs, such as angiopoietin-2, vascular endothelial growth factor (VEGF), and angiopoietin-like 4, facilitate the passage of metastatic cancer cells through the vessel wall [Id., citing Pastorek, J. and Pastorekova, S. Semin. Cancer Biol. (2015) 31: 52-64].

Glycolysis. The anoxic microenvironment is beneficial for glycolysis and lactic acid production by key enzymes of glycolysis and lactate dehydrogenase A (LDH-A); the excess production of lactic acid results in an acidic pH. Moreover, HIF can reversely convert carbon dioxide and water produced by the activation of carbonic anhydrase IX or XII into HCO₃⁻, which diffuses out of the cell membrane, resulting in excess HCO₃⁻ in the TME and a decrease in pH [Id., citing Harris, A L. Nat. Rev. Cancer (2002) 2: 38-47]. A large number of studies have concluded that the decreased intracellular pH of endosomes and lysosomes in tumor cells may assist in metastasis by activating proteases [Id., citing Nelson, D A, et al. Genes Dev. (2004) 18: 2095-107; Pilon-Thomas, S. et al. Cancer Res. (2016) 76: 1381-1390].

Reactive oxygen species. The level of reactive oxygen species (ROS) has been shown to be increased in cancer cells exposed to hypoxia [Id., citing Zhu, X. and Zuo, L. Cell Death Dis. (2013) 4: e787]. The reduction in oxygen utilization decreases the passage of electrons through the mitochondrial complex by the electron transport chain (ETC), allowing electrons to leak from the ETC, thus leading to the overproduction of ROS [Id., citing Guzy, R D, et al. Cell Metab. (2005) 1: 401-408]. Moreover, the excessive production of ROS alters genomic stability and impairs DNA repair pathways [Id., citing Nita, M. and Grzybowski, A. Oxidative Med. Cell Longev. (2016) 2016: 3164734]. ROS can affect cell survival or apoptosis via oxidative stress, thus resulting in enhanced cytotoxicity and apoptosis [Id., citing Bridge, G., et al. Cancers (Basel) (2014) 6: 1597-1614]. At high concentrations (10-30 μm), ROS can damage cellular biomolecules, such as proteins, DNA, and RNA, and cause mutations that promote cancer in normal cells or multidrug resistance (MDR) in cancer cells [Id., citing Syu, J P, et al. Oncotarget (2016) 7: 14659-14672]. However, most cancer cells still survive under internal oxidative stress, hence avoiding apoptosis and developing resistance to chemotherapy. Exposure to elevated levels of ROS can lead to cancer cell resistance by the activation of redox-sensitive transcription factors such as NF-κB, nuclear factor (erythroid-derived 2)-like factor 2 (Nrf2), c-Jun, and HIF-1α [Id., citing Shen, Y., et al. Exp. Cell Res. (2015) 334: 207-218]. Subsequently, the activation of these genes enhances the activation of the antioxidant system and promotes the expression of cell survival proteins. In addition, ROS facilitate the transition from apoptosis to autophagy in methotrexate-resistant choriocarcinoma jeg-3 cells, enabling the survival of cells to methotrexate [Id., citing Corzo, C A, et al. J. Exp. Med. (2010) 207: 2439-2453]. ROS can also stimulate the differentiation of cancer stem cells, thus promoting epithelial-mesenchymal transition (EMT) and inducing metabolic reprogramming involved in the resistance of cancer cells.

Epithelial-mesenchymal transition (EMT). EMT is a key process in the metastasis and colonization of cancer cells from the primary tumor to distant organs. HIF has a direct regulatory effect on EMT-related proteins, such as zinc finger E-box binding homeobox 1, Snail and Twist [Zhuang, Y., et al. MedComm. (2023) 4 (1): e403, citing Yang, M H, et al. Nat. Cell Biol. (2008) 10 (3): 295-305; Zhang, W., et al. PLOS One (2015) 10 (6): e0129603; Xi, Y., et al. Mol. Cancer (2022) 21 (1): 145]. At the same time, HIF can also modulate microRNA (miRNA) to promote the cellular EMT process [Id., citing Xi, Y., et al. Mol. Cancer (2022) 21 (1): 145; Li, H., et al. Gastroentrology (2017) 153 (20: 505-520; Xu, Q., et al. Mol. Cancer (2017) 16 (1): 103; Xing, S., et al. Mol. Cancer (2021) 20 (1): 9].

Immunosuppression. Hypoxic stress causes immunosuppression by controlling angiogenesis and favoring immune suppression and tumor resistance. Macrophages constitute a principal component of the immune infiltrate in solid tumors by differentiating into tumor-associated macrophages (TAMs), which have been found to be preferentially located in tumor hypoxic areas [Jing, X., et al. Molecular Cancer (2019) 18: 157, citing Mantovani, A. et al. Trends Immunol. (2002) 23: 549-55]. Tumor-derived cytokines are able to convert TAMs into polarized type 2, or M2, macrophages with more immunosuppressive activities, resulting in tumor progression. Myeloid-derived suppressor cells (MDSCs) can directly promote immune tolerance [Id., citing Noman, M Z, et al., J. Exp. Med. (2014) 211: 781-790]. In hypoxic zones, HIF-1 directly regulates the function and differentiation of MDSCs, and such tumor-derived MDSCs are more immunosuppressive compared with splenic MDSCs. The upregulation of the expression of programmed death-ligand 1 (PD-L1) under hypoxia has been shown [Id., citing Barsoum, I B, et al. Cancer Res. (2014) 74: 665-674]. Further evidence supports the claim that HIF-1 is a major regulator of PD-L1 mRNA and protein expression. HIF-1 regulates the expression of PD-L1 by binding directly to a hypoxia response element in the PD-L1 proximal promoter [Id., citing Noman, M Z, et al. J. Exp. Med. (2014) 211: 781-790]. The originally elevated immunosuppressive function of tumor-derived MDSCs under hypoxia was found to be abrogated following PD-L1 blockade. Along with PD-L1 blockade, the hypoxia-mediated upregulation of IL-6 and IL-10 in MDSCs was significantly attenuated [Id., citing Saggar, J K, et al. Front. Oncol. (2013) 3: 154].

At present, immunotherapeutic strategies triggering antitumor immunity are not effective because of diverse mechanisms of tumor escape from immunosurveillance. The antibody blockade of the T-cell immune checkpoint receptors PD-1 and CTLA-4 was poor in some tumors because T cells were sparse or absent in the TME; the hypoxia-driven modulation of T-cell exclusion and apoptosis help maintain this state. Although T cells can enter hypoxic tumors, the hypoxia-mediated acidification of the extracellular milieu blocks the capacity of T cells to expand or perform cytotoxic effector functions.

Hypoxia leads to a decreased pH in the TME. Since some chemotherapeutic drugs currently used in clinical practice are pH dependent in terms of their intracellular targets, changes in the intracellular pH gradient result in decreased drug accumulation in tumor cells, thereby greatly reducing the efficacy of chemotherapeutic drugs and eventually leading to drug resistance.

Defective apoptosis Anticancer treatments act in part by inducing apoptosis [Id., citing Maddika, S, et al. Drug Resist. Updat. (2007) 10: 13-29; Enari, M. et al. Nature (1998) 391: 43-50]. Tumor cells always alter their metabolism to ensure survival and evade host immune attack to proliferate. Under hypoxic conditions, nonadaptive cancer cells undergo apoptosis via HIF-1- and P53-dependent mechanisms.

Tumor immunology has advanced in the last 20 years and improved the understanding of how tumors avoid attack from the immune system. These achievements have lead to new therapeutic modalities to enhance anti-tumor immunity in cancer patients.

Three phases of tumor growth are recognized in tumorigenesis. The first, the elimination phase, is the stage in which the immune system recognizes and destroys potential tumor cells by immune surveillance. If elimination is not completely successful, the second phase of tumor growth is an equilibrium phase in which tumor cells undergo changes or mutations that aid their survival as a result of the selection pressure imposed by the immune system. During the equilibrium phase, cancer immunoediting continuously shapes the properties of the tumor cells that survive. In the third phase, the escape phase, tumor cells that have acquired the ability to elude the attentions of the immune system and grow unimpeded become clinically detectable.

Oncogenesis is derived from genetic or epigenetic aberrations, which produce proteins that differ from normal proteins, as the source of tumor-specific antigens. These mutation-related neoantigens could be recognized by adaptive immune system to induce immune attack against the tumor. T or B cell clones can be expanded when they are exposed to neoantigens to mount cellular or humoral immune response toward tumor. The knowledge of tumor immunology lead to multi-modalities of immunotherapy, including usage of (1) monoclonal antibodies against immune checkpoint inhibitors, such as PD-(L) 1, CTLA-4, TIGIT, LAG3, TIM3 and other cell surface markers; (2) antibody drug conjugate (ADC) to use monoclonal antibody to carry cytotoxic agents to tumor; (3) cell therapy, including CAR-T, TCR, NKs, or TILs; (4) DNA or mRNA therapy to generate cancer vaccine, etc.

A major limitation of current approaches is that all the targets of immunotherapy are toward the tumor-associated antigens, which are encoded by unmutated genes that may also be expressed in normal tissues, such as germline antigens; tissue differentiation antigens; human endogenous retrovirus; overexpressed tumor antigens. Although tumor associated antigens do have a preferential expression in tumor compared to normal tissues, potent immunotherapy targeting tumor-associated antigens could still cause significant toxicity by the off-target effect. Examples are immunotherapy agents targeting against CD19, CD20, or BCMA by monoclonal antibody, ADC or cell therapy based on TAA. Significant adverse effects are frequently observed and sometimes even severe enough to be lethal.

Overcoming the off-target effect requires the development an effective immunotherapy by targeting tumor-specific antigens, which are not expressed in normal tissues. However, identification of tumor-specific neoantigens has been a major hurdle. If they are identified in a cancer patient, a variety of new therapies can then be derived from the tumor-specific antigen based on the existing technology. Tremendous efforts have been devoted to this. With the use of next generation sequencing (NGS), it is now relatively straightforward to identify all mutations present in tumor cells. It was estimated that a cancer cell could have 30-100 mutations depending on cancer types, that are different from the sequences of the normal genome [Vogelstein, B. et al. Science (2013) 339: 1546-1558]. However, identification of mutations for a cancer does not mean that all these mutations would encode tumor-specific antigens even after excluding the single nucleotide polymorphism. Due to the restriction that a tumor-specific antigen peptide needs to be associated with the MHC molecule in order to interact with T cell receptor (TCR) in antigen-presenting cells (macrophages or dendritic cells) or during the process of targeting tumor cells by cytotoxic T cells (FIG. 1), not all mutation-generated neo-peptides can serve as a neoantigen to trigger anti-tumor immunity.

The complexity can be calculated numerically as follows. One point mutation can result in 10 peptides that include a point mutation at various locations within the peptide. As the number times the number of mutations in a tumor and times the potential number of MHC molecules different in a population, the number of potential tumor-specific mutations that can be derived from one point mutation will be dauntingly over thousands. Although computer algorithms have been developed to predict the possibility of each mutation to serve as a tumor-specific antigen to build a cancer vaccine, the process is tedious and unreliable. Success has been very limited using this approach to develop a cancer vaccine, except in cancers with high mutation rates, such as melanoma.

Another approach pioneered by Rosenberg's group in National Cancer Institute was to isolate Tumor-Infiltrated Lymphocytes (TILs), which are supposed to include T cells that recognize tumor-specific antigens [see, e.g., Rosenberg, S A et al. J. Intl Cancer Instit. (1994) 86 (15): 1159-66]. Using this approach and combined with the knowledge of tumor mutations derived from NGS and extensive biological testing, they identified previously unrecognized tumor-specific antigens. However, since TILs are a mixed population with some TILs as T-exhaust cells, meaning that these cells are unable to have an activity of anti-tumor immunity, hence in an “exhausted” status. T cell receptors from T-exhaust would need to be excluded due to lack of anti-tumor activity. Parkhurst et al. reported 83% of patients with gastro-intestinal cancer have recognizable neoantigens. But they are encoded by only 1.6% of somatic mutations in autologous tumor cells. (Parkhurst, Cancer Discov. (2019) 9: 1022-35). More strikingly, among the 124 neoantigens targeted by active TILs, all but one of the neoantigenic determinants were unique. This finding shows that the majority of common epithelial cancers can elicit immune recognition and open the possibilities for cell-based immunotherapies for patients with these GI malignancies. However, because the unique nature of each neoantigen not shared among patients, an individualized approach would be necessary and it is not likely to develop a universal cancer vaccine, or cell therapy, for most of the cancer patients.

Hence there is a strong need for a new methodology to identify tumor-specific mutations that have the capability of mounting an effective anti-tumor immunity to build a cancer vaccine, or the corresponding T cell comprising a TCR specific for a tumor cancer antigen that can be used directly as a cell therapy.

The advantage of this individualized approach is that these tumor-specific neoantigens are truly “novel antigen” specific to tumor and not shared with any normal organ. Mounting an immunity against these tumor-specific neoantigens with a cancer vaccine or cell therapy would not be likely to have an off-target effect. It is expected to be much safer and effective.

### SUMMARY OF THE INVENTION

According to one aspect, the present disclosure provides an autologous cellular immunotherapy for treating a cancer comprising, in order: (a) administering to a subject with a cancer (i) a Tumor Necrosis-Inducing Agent (TUNIA) to induce tumor necrosis and reduce tumor burden and (ii) a checkpoint inhibitor; (b) isolating peripheral blood mononuclear cells (PBMCs) comprising a CD4+ T cell population, a CD8+ T cell population, a Natural Killer (NK) cell population, and an NK-T cell population from peripheral blood of the subject by density gradient centrifugation; (c) extracting RNA and DNA from the CD4+ T cell population and CD8+ T cell population; (d) preparing a sequencing library from each of the RNA or DNA sample by (1) amplification to yield a pool of appropriately sized target sequences; and (2) the addition of sequencing adapters that later will interact with a next generation sequencing (NGS) platform; (e) amplifying the sequence library by polymerase chain reaction (PCR) to yield a library comprising a collection of specifically sized DNA fragments; (f) loading the library onto a sequencer and performing parallel sequencing using a next generation sequencing (NGS) platform; (g) after sequencing is complete, filtering the reads for quality, amplicon size, and agreement between paired ends; (h) assembling and aligning the reads to a reference genome for a T cell receptor comprising 2 protein chains; (i) identifying expanded clonal variants of complementarity-determining region-3 (CDR3) of the T cell receptor comprising 2 protein chains by comparing the reads (assembled or raw) to the sequence of the CDR3 of a reference TCR sequence or to reads from another sample to identify variants; (j) expanding in vitro the PBMCs from the peripheral blood of the patient comprising the expanded clonal variants of CDR3; and (k) administering to the subject by infusion the PBMCs after a polyclonal expansion in (j)

According to some embodiments of the autologous cellular immunotherapy, the administering is for at least two months.

According to some embodiments, the autologous cellular immunotherapy further comprises flow cytometry analysis of a sample of the PBMCs in step (b) and after expansion of the PBMCs in step (j) with CD3, CD4, CD8, CD45RO, CCR7 and CD56 markers.

According to some embodiments, the flow cytometry analysis characterizes the cell populations comprising naïve memory cells, central memory cells, effector memory cells, effector cells, Natural Killer cells, and NK-T cells in the PBMCs.

According to some embodiments, the clonal variants of CDR3 that appear after TATE treatment recognize a tumor neoantigen.

According to some embodiments, after ex vivo expansion in step (j), (a) the percentage of the total PBMC cell population represented by each of the monocyte subpopulation, the NK cell subpopulation and the CD4 cell subpopulation was reduced compared to its percentage before ex vivo expansion; and (b) the percentage of the total PBMC cell population represented by each of the B cell subpopulation, the CD8+ cell population and the NKT cell population increased compared to its percentage before ex vivo expansion.

According to some embodiments, after ex-vivo expansion for 10 days, the CD8+ cell subpopulation comprising cytotoxic T cells and the NKT cell subpopulation dominate the PBMC cell population while the CD4+ cell subpopulation comprising an immunosuppressive Treg subpopulation is reduced compared to its percentage before ex vivo expansion.

According to some embodiments, the tumor necrosis inducing agent (TUNIA) step (a) comprises an in vivo immunizing effect.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic showing how a T cell receptor (TCR) interacts with the neo-antigen peptide, which fits in a groove of the MHC molecule. Two helices in the MHC molecule are essential for interaction with the CDR1 and CDR2 of the TCR to form a tertiary complex.

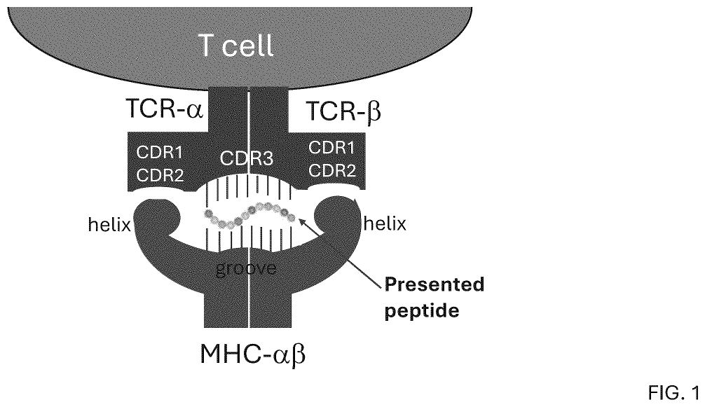

FIG. 2 is a flow chart showing a procedure for blood sample collection and processing.

FIG. 3 is a schematic diagram of the methodology from collection of blood samples to identification of the complementarity determining region-3 of a TCR.

FIG. 4 is a histogram of the copy number of newly appeared clones after TATE treatment (only those with copy number >500 are shown).

FIG. 5 is a scatter plot for folds of expansion (in green) of CDR3 vs. CDR3 copy numbers after TATE treatment.

FIG. 6 is a schematic depicting the effect of expansion of peripheral blood mononuclear cells after TATE and subsequent ex vivo expansion.

FIG. 7 is a diagram depicting the process of the described autologous cell therapy platform approach for solid tumor patients.

FIG. 8 shows the expansion of cells after ex vivo expansion. Total cell count was 5.0×10E6 at inoculation and became 5.58×10E6 after 5 days of culture. Then the cell growth entered into a rapid proliferation phase and achieved 1.43×10E8 or a 28.6-fold expansion compared to the starting cell count.

FIG. 9A and FIG. 9B showed the distribution of various subpopulations of PBMCs for a patient previously treated with TUNIA before and after ex vivo expansion. Flow cytometry analysis was conducted to determine the percentage of B cells, monocytes, NK cells, CD4+, CD8+ and NKT cells using their specific markers. FIG. 9A shows that three subpopulations of cells, namely monocytes, NK cells and CD4 cells, exhibited a dramatic reduction in their percentages during the process of ex vivo expansion. In contrast, the subpopulation of B cells, CD8+ and NKT cells showed significant expansion. Without being limited by theory, the mechanism of this preferential expansion in these populations could be due to the immunization effect in vivo by TUNIA. Consistent with the expansion of CDR3 clones as described in TABLE 1 and FIG. 4, more tumor-specific cytotoxic T cells were generated in vivo, which are also in an active proliferative stage compared to the non-tumor targeted T cells and achieved a preferential expansion during culture. After 10 days of ex vivo expansion, CD8+ cells became the dominant cell population comprising about 72.0%, whereas NKT cells are the second most prominent population, comprising about 15.5%. As shown in FIG. 9B, the effect of CD8+ dominance in the product was even more prominent when the absolute cell count of each population was calculated. This pattern of cell distribution is considered a highly desirable cell profile, as the CD8+ population includes most cytotoxic T cells against the tumor, and NKT cells are also expected to play an important role against the tumor. In contrast, the population of CD4+ cells contain the Treg subpopulation, which commonly has an immune suppressive function to regulate the activity of CD8+ cytotoxic T cells. The reversal of the ratio for CD4+/CD8+ in the ex vivo expanded product resulting from the process indicates a more favorable product for anti-tumor cell therapy.

### DETAILED DESCRIPTION OF THE INVENTION

### Definitions

As used in the specification and the appended claims, the singular forms “a,” “an” and “the” include plural referents unless the context clearly dictates otherwise.

The term “about” as used herein when referring to a measurable value such as an amount, a temporal duration, and the like, is meant to encompass variations of +20%, +10%, +5%, +1%, +0.9%, +0.8%, +0.7%, +0.6%, +0.5%, +0.4%, +0.3%, +0.2% or +0.1% from the specified value, as such variations are appropriate to perform the disclosed methods.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer., According to some embodiments, to A without B (optionally including elements other than B).; According to some embodiments, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a polypeptide “comprises” an amino acid sequence when the amino acid sequence might be part of the final amino acid sequence of the polypeptide. Such a polypeptide can have up to several hundred additional amino acids residues (e.g. tag and targeting peptides as mentioned herein). “Consisting essentially of” means excluding other components or steps of any essential significance. Thus, a composition consisting essentially of the recited components would not exclude trace contaminants and pharmaceutically acceptable carriers. A polypeptide “consists essentially of” an amino acid sequence when such an amino acid sequence is present with eventually only a few additional amino acid residues. “Consisting of” means excluding more than trace elements of other components or steps. For example, a polypeptide “consists of” an amino acid sequence when the polypeptide does not contain any amino acids but the recited amino acid sequence.

The term “adaptor” or “adapter” as used herein refers to nonenzymatic proteins that form physical links between members of a signaling pathway, particularly between a receptor and other signaling proteins. They recruit members of the signaling pathway into functional protein complexes.

The term “adjuvant effect” as used herein refers to immune-enhancing effects that allow a more effective immune response. Without being limited by theory, an adjuvant effect may include one or more of the following immune functional activities: enhanced local reaction at a site of administration; induction of the release of inflammatory cytokines; or interaction with innate immune biosensors (e.g., PRRs, including TLRs) leading to PRR signaling. [See, e.g., Chuang, Y-C et al. Front. Immunol. (2020) 11: 1075].

As used herein, the term “antibody” includes, by way of example, both naturally occurring and non-naturally occurring antibodies. Specifically, the term “antibody” includes polyclonal antibodies and monoclonal antibodies, and fragments thereof. Furthermore, the term “antibody” includes chimeric antibodies and wholly synthetic antibodies, and fragments thereof.

As used herein, the term “antibody” is used in the broadest sense and encompasses various antibody structures, including but not limited to monoclonal antibodies, polyclonal antibodies, antibody fragments, chimeric antibodies and wholly synthetic antibodies as long as they exhibit the desired antigen-binding activity. In nature, antibodies are serum proteins the molecules of which possess small areas of their surface that are complementary to small chemical groupings on their targets. These complementary regions (referred to as the antibody combining sites or antigen binding sites) of which there are at least two per whole antibody molecule, and in some types of antibody molecules ten, eight, or in some species as many as 12, may react with their corresponding complementary region on an antigen (the antigenic determinant or epitope) to link several molecules of multivalent antigen together to form a lattice. The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, K and 2; individual molecules of immunoglobulin generally are only one or the other. In mammals, there are five classes of antibodies, IgA, IgD, IgE, IgG, and IgM, each with its own class of heavy chain. All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human VL chains with the heavy chain variable region (VH) domain of a mouse monoclonal antibody reactive with an antigen of interest; (ii) selecting half-human Fabs on that antigen (iii) using the selected V L genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1 antibody molecule in the mouse myeloma.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques.

An antibody may be from any species. The term antibody also includes binding fragments of the antibodies of the invention. Binding fragments of an antibody can be produced by recombinant DNA techniques, or by enzymatic or chemical cleavage of intact antibodies. Exemplary fragments include Fv, Fab, Fab′, single stranded antibody (svFC), dimeric variable region (Diabody) and di-sulfide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. See, for example, Bowie et al. Science 253: 164 (1991), which is incorporated by reference in its entirety. An antibody other than a “bispecific” or “bifunctional” antibody is understood to have each of its binding sites identical.

The term “antibody construct” as used herein refers to a polypeptide comprising one or more the antigen-binding portions of the invention linked to a linker polypeptide or an immunoglobulin constant domain. Linker polypeptides comprise two or more amino acid residues joined by peptide bonds and are used to link one or more antigen-binding portions. Such linker polypeptides are well known in the art [see e.g., Holliger, P., et al. (1993) Proc. Natl. Acad. Sci. USA 90: 6444-6448; Poljak, R. J., et al. (1994) Structure 2: 1121-1123]. An immunoglobulin constant domain refers to a heavy or light chain constant domain. Human IgG heavy chain and light chain constant domain amino acid sequences are known in the art. Antibody portions, such as Fab and F(ab′) 2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

The term “antibody-drug conjugate” or “ADC” as used herein refers to antibodies (e.g., monoclonal antibodies, mAbs) linked to a cytotoxic agent designed to induce target cell death in order to reduce systemic exposure and therefore toxicity of the cytotoxic agent. The linker should be stable in circulation but release the cytotoxic agent once it is delivered to target cells. The unique antigenic target of the antibody component needs to have high expression in the tumor and no or low expression in healthy cells; it should be displayed on the surface of the tumor cell to be available to the circulated antibody; it should possess internalization properties to facilitate the ADS to transport into the cell which will in turn enhance the efficacy of the cytotoxic agent; or exert a “bystander effect.”

The term “antigen” as used herein, is meant to refer to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 7, 8, 9, 10, 11, 12, 13, 14, or 15 amino acids. The term includes polypeptides which include modifications, such as deletions, additions and substitutions (generally conservative in nature) as compared to a native sequence, as long as the protein maintains the ability to elicit an immunological response, as defined herein. These modifications may be deliberate, as through site-directed mutagenesis, or may be accidental, such as through mutations of hosts which produce the antigens.

The term “antigen presentation” as used herein, generally refers to the display of antigen on the surface of a cell, e.g., in the form of peptide fragments bound to MHC molecules.

As used herein, the term “antigen presenting cell (APC)” refers to a class of cells capable of displaying on its surface (“presenting”) one or more antigens in the form of peptide-MHC complex recognizable by specific effector cells of the immune system and thereby inducing an effective cellular immune response against the antigen or antigens being presented. Examples of professional APCs are dendritic cells and macrophages, though any cell expressing MHC Class I or II molecules can potentially present peptide antigen. An APC can be an irradiated population of PBMCs. An APC can be an “artificial APC,” meaning a cell that is engineered to present one or more antigens. Before a T cell can recognize a foreign protein, the protein has to be processed inside an antigen presenting cell or target cell so that it can be displayed as peptide-MHC complexes on the cell surface.

As used herein the term “antigen processing” refers to the intracellular degradation of foreign proteins into peptides that can bind to MHC molecules for presentation to T cells.

The terms “apoptosis” or “programmed cell death” refer to a highly regulated and active process that contributes to biologic homeostasis comprising a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways.

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to death domain (DD) containing receptors like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits inhibitor of apoptosis (IAP) proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome c is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation, can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. [Loberg, R D, et al., J. Biol. Chem. (2002) 277 (44): 41667-41673]. It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “artificial intelligence” or “AI” refers to the simulation of human intelligence by machines. AI programming includes the following cognitive skills: learning (acquiring data and creating rules for how to turn it into actionable information (algorithms), that provide step-by-step instructions for how to complete a specific task; reasoning (choosing the right algorithm); self-correction (fine-tuning algorithms to provide the best result); and creativity using AI techniques. Technologies that fall under the umbrella of AI include machine learning and deep learning.

The term “binding” and its various grammatical forms means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “binding specificity” as used herein involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

As used herein, the term “biomarker” (or “biosignature”) refers to a peptide, protein, nucleic acid, antibody, gene, metabolite, or any other substance used as an indicator of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “cancer stem cells” as used herein refers to a small number of cells in a tumor with the ability to self-renew and drive tumorigenesis. The stem cell theory of cancer fostering the idea that cancer is primarily driven by a smaller population of stem cells has important implications. For instance, if a therapy does not kill the cancer stem cells within a tumor, chances are that the cancer stem cells will drive the tumor to grow back, often with resistance to the previously used therapy.

As used herein, the term “cell growth” is the process by which cells accumulate mass and increase in physical size. There are many different examples in nature of how cells can grow. In some cases, cell size is proportional to DNA content. For instance, continued DNA replication in the absence of cell division (called endoreplication) results in increased cell size. Megakaryoblasts, which mature into granular megakaryocytes, the platelet-producing cells of bone marrow, typically grow this way. By a different strategy, adipocytes can grow to approximately 85 to 120 μm by accumulating intracellular lipids. In contrast to endoreplication or lipid accumulation, some terminally differentiated cells, such as neurons and cardiac muscle cells, cease dividing and grow without increasing their DNA content. These cells proportionately increase their macromolecule content (largely protein) to a point necessary to perform their specialized functions. This involves coordination between extracellular cues from nutrients and growth factors and intracellular signaling networks responsible for controlling cellular energy availability and macromolecular synthesis. Perhaps the most tightly regulated cell growth occurs in dividing cells, where cell growth and cell division are clearly separable processes. Dividing cells generally must increase in size with each passage through the cell division cycle to ensure that a consistent average cell size is maintained. For a typical dividing mammalian cell, growth occurs in the G1 phase of the cell cycle and is tightly coordinated with S phase (DNA synthesis) and M phase (mitosis). The combined influence of growth factors, hormones, and nutrient availability provides the external cues for cells to grow. [Guertin, D. A., Sabatini, D. M., “Cell Growth,” in The Molecular Basis of Cancer (4th Edn) Mendelsohn, J. et al Eds, Saunders (2015), 179-190].

As used herein, the term “cell proliferation” is meant to refer to the process that results in an increase of the number of cells and is defined by the balance between cell divisions and cell loss through cell death or differentiation.

As used herein, the term “chemokine” is meant to refer to a class of chemotactic cytokines that orchestrate migration and positioning of immune cells within the tissues. Chemokines bind to seven transmembrane G protein-coupled receptors that trigger intracellular signaling that drives cell polarization, adhesion, and migration [Vilgelm, A E and Richmond, A. Front. Immunol. (2019) doi.org/10.3389/fimmu.2019.00333, citing Griffith, J W et al. Annu. Rev. Immunol. (2014) 32: 659-702; Nagarsheth, N. et al. Nat. Rev. Immunol. (2017) 17: 559-72]. They are divided into four families based upon structure: CXC, CC, CX3C, and C chemokines. The receptors follow a similar nomenclature system, based upon the family of chemokines to which they bind. In addition, there is a family of atypical chemokine receptors that do not directly couple to G proteins but are reported to have a variety of roles in development, homeostasis, inflammatory disease, infection, and cancer [Id., citing Nibbs, R J, Graham, GJ. Nat. Rev. Immunol. (2013) 13: 815-29].

The term “class switching”, “isotype switching” or “class switch recombination” as used herein refers to a somatic gene recombination process in activated B cells that replaces one heavy chain constant region with one of a different isotype, switching the isotype of antibodies from IgM to IgG, IgA or IgE. This affects the antibody effector functions but not their antigen specificity.

As used herein, the term “cognate help” is meant to refer to a process that occurs most efficiently in the context of an intimate interaction with a helper T cell.

The term “complementarity-determining region” or “CDR” as used herein refers to immunoglobulin (Ig) hypervariable domains that determine specific antibody binding. The variable (V) domains of the TCR are structurally similar to antibody V domains. Each TCR V domain (α and β) contains three complementarity determining region (CDR) loops that combine to form the TCR binding interface.

As used herein, the term “contact” and its various grammatical forms is meant to refer to a state or condition of touching or of immediate or local proximity. Contacting a composition to a target destination may occur by any means of administration known to the skilled artisan.

The term “costimulation” as used herein refers to the second signal required for completion of lymphocyte activation and prevention of anergy, which is supplied by engagement of CD28 by CD80 and CD86 (T cells) and of CD40 by CD40 Ligand (B cells).

The term “costimulatory molecule” as used herein refers to molecules that are displayed on the cell surface that have a role in enhancing the activation of a T cell that is already being stimulated through its TCR. For example, HLA proteins, which present foreign antigen to the T cell receptor, require costimulatory proteins which bind to complementary receptors on the T cell's surface to result in enhanced activation of the T cell. The term “co-stimulatory molecules” as used herein refers to highly active immunomodulatory proteins that play a critical role in the development and maintenance of an adaptive immune response (Kaufman and Wolchok eds., General Principles of Tumor Immunotherapy, Chpt 5, 67-121 (2007)). The two signal hypothesis of T cell response involves the interaction between an antigen bound to an HLA molecule and with its cognate T cell receptor (TCR), and an interaction of a co-stimulatory molecule and its ligand. Specialized APCs, which are carriers of a co-stimulatory second signal, are able to activate T cell responses following binding of the HLA molecule with TCR. By contrast, somatic tissues do not express the second signal and thereby induce T cell unresponsiveness (Id.). Many of the co-stimulatory molecules involved in the two-signal model can be blocked by co-inhibitory molecules that are expressed by normal tissue (Id.). In fact, many types of interacting immunomodulatory molecules expressed on a wide variety of tissues may exert both stimulatory and inhibitory functions depending on the immunologic context (Id.). As used herein the term “co-stimulatory receptor” is meant to refer to a cell surface receptor on naïve lymphocytes through which they receive signals additional to those received through the antigen receptor, and which are necessary for the full activation of the lymphocyte. Examples are CD30 and CD40 on B cells, and CD27 and CD28 on T cells.

As used herein, the term “coverage” in reference to NGS refers to the average number of reads that align to, or “cover” known reference basis. The sequencing coverage level determines whether variant discovery can be made with a certain degree of confidence at particular base positions. Coverage equals read count multiplied by the read length and divided by the total genome size. At a higher level of coverage, each base is covered by a greater number of aligned sequence reads, and mutations at the base level compared to a reference sample can be determined. In some embodiments, a reference sample may be a pooled reference sample. In some embodiments, the pooled reference sample may be a pooled normal reference sample.

The term “cross-dressing” as used herein refers to a third pathway for cross-presentation. In cross-dressing, dendritic cells acquire preformed MHC class I molecules in complex with antigens from other cells by the process of trogocytotis (meaning the transfer of cell membrane patches or individual proteins between cells [Yewdell, J W and Dolan, BP, “Cross-dressers turn on T cells.” Nature (2011) 471 (7340): 581-82, citing Joly, E. and Hudrisier, D. Nature Immunol. (2003) 4: 8:15; Herrera O B et al. J. Immunol. (2004) 173: 4828-37] or through gap junctions. This allows antigen presentation by acceptor dendritic cells to occur immediately, without any processing. Cross-dressing is used to activate memory T cells, but not naïve T cells, in response to viral infection [Id., citing Wakins, L M and Bevan, MJ. Nature (2011) 471: 629-32].

The term “cross-presentation” as used herein refers to a process by which proteins taken up by dendritic cells from the extracellular milieu can give rise to peptides presented by MHC class I molecules. It enables antigens from extracellular sources to be presented by MHC class I molecules and to activate CD8 T cells.

The term “cross-priming” as used herein refers to activation of CD8 T cells by dendritic cells in which the antigenic peptide presented by MHC class I molecules is derived from an exogenous protein (i.e., by cross-presentation), rather than produced within the dendritic cells directly (compare direct presentation).

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines. Non-limiting examples of cytokines include e.g., IL-1, IL-2, IL-3, IL-4, IL-5, IL-6, IL-7, IL-8, IL-9, IL-10, IL-11, IL-12/IL-23 P40, IL13, IL-15, IL-15/IL15-RA, IL-17, IL-18, IL-21, IL-23, TGF-β, IFNγ, GM-CSF, Groα, MCP-1 and TNF-α.

The term “cytotoxic T lymphocytes” (CTLs) as used herein, is meant to refer to effector CD8+ T cells. Cytotoxic T cells kill by inducing their targets to undergo apoptosis. They induce target cells to undergo programmed cell death via extrinsic and intrinsic pathways.

The term damage-associated molecular patterns” or “DAMPS” as used herein refers to molecules released by stressed or dying cells that bind to pattern recognition molecules (PRMs) and induce inflammation.

The term “deep learning” as used herein, a subset of machine learning, is based on our understanding of how the brain is structured and involves use of artificial neural networks.

The term “dendritic cells (DC)” as used herein refers to professional antigen presenting cells, which induce naïve T cell activation and effector differentiation. [Patente, T A, et al., Frontiers Immunol. (2019) doi.org/10.3389/fimmu.2018.03176]. Human DC are identified by their high expression of major histocompatibility complex (MHC) class II molecules (MHC-II) and of CD11c, both of which are found on other cells, like lymphocytes, monocytes and macrophages [Id., citing Carlens J, et al. J Immunol. (2009) 183: 5600-5607; Drutman S B, et al. J Immunol. (2012) 188: 3603-3610; Hochweller K, et al. Eur J Immunol. (2008) 38: 2776-2783; Huleatt J W, Lefrançois L. J Immunol. (1995) 154: 5684-93; Rubtsov A V, et al. Blood (2011) 118: 1305-1315; Probst H C, et al. Clin Exp Immunol. (2005) 141: 398-404; Vermaelen K, Pauwels R. Cytometry (2004) 61A: 170-177]. DC express many other molecules which allow their classification into various subtypes. Although some of the DC subtypes were originally described as macrophages, DC and macrophages have distinct characteristics [Id., citing Delamarre L, Science (2005) 307: 1630-1634; Geissmann F, et al. Science (2010) 327: 656-661; van Montfoort N, et al. Proc Natl Acad Sci USA. (2009) 106: 6730-6735] and ontogeny, so that, currently, little doubt remains that they belong to distinct lineages [Id., citing Haniffa M, et al. (2013) 120: 1-49; Hashimoto D, et al. Immunity (2013) 38: 792-804; Hettinger J, et al. Nat Immunol. (2013) 14: 821-830; McGovern N, et al. Immunity (2014) 41: 465-477; Naik S H, et al. Nature (2013) 496: 229-232; Schulz C, et al. Science (2012) 336: 86-90; Schraml B U, et al. Cell (2013) 154: 843-858; Wang J, et al. Mol Med Rep. (2017) 16: 6787-6793; Yona S, et al. Immunity (2013) 38: 79-91]. DCs are found in two different functional states, “mature” and “immature”. These are distinguished by many features, but the ability to activate antigen-specific naïve T cells in secondary lymphoid organs is the hallmark of mature DCs [Id., citing Hawiger D, Inaba K, et al. J Exp Med. (2001) 194: 769-79; Steinman R M, et al. Ann NY Acad Sci. (2003) 987: 15-25; Worbs T, et al. Nat Rev Immunol. (2017) 17: 30-48]. DC maturation is triggered by tissue homeostasis disturbances, which is detected by the recognition of pathogen-associated molecular patterns (PAMP) or damage-associated molecular patterns (DAMPs) [Id., citing Hemmi H, et al. Chem Immunol Aller. (2005) 86: 120-135, Cerboni S, et al. Adv Immunol. (2013) 120: 211-237]. Maturation turns on metabolic, cellular, and gene transcription programs allowing DCs to migrate from peripheral tissues to T-dependent areas in secondary lymphoid organs, where T lymphocyte-activating antigen presentation may occur [Id., citing Alvarez D, et al. Immunity (2008) 29: 325-342; Dong H, Bullock T N J. Front Immunol. (2014) 5: 24; Friedl P, Gunzer M. Trends Immunol. (2001) 22: 187-191; Henderson R A, et al. J Immunol. (1997) 159: 635-643; Randolph G J, et al. Nature Rev Immunol. (2005) 5: 617-628 Imai Y, et al. Histol Histopathol. (1998) 13: 469-510]. During maturation, DCs lose adhesive structures, reorganize the cytoskeleton and increase their motility [Id., citing Winzler C, et al. J Exp Med. (1997) 185: 317-328). DC maturation also leads to a decrease in their endocytic activity but increased expression of MHC-II and co-stimulatory molecules [Id., citing Reis e Sousa C. Nature Rev Immunol. (2006) 6: 476-483; Steinman R M. Annu Rev Immunol. (2012) 30: 1-22; Trombetta, E S. and Mellman I. Annu Rev Immunol. (2005) 23: 975-1028]. Mature DCs express higher levels of the chemokine receptor CCR7 [Id., citing Forster R, et al. Cell (1999) 99: 23-33; Ohl L, et al. Immunity (2004) 21: 279-288; Sallusto F, et al. Eur J Immunol. (1998) 28: 2760-2769; Steinman R M. The control of immunity and tolerance by dendritic cell. Pathol Biol. (2003) 51: 59-60] and secrete cytokines, essential for T-cell activation [Id., citing Reis e Sousa C. Nature Rev Immunol. (2006) 6: 476-483; Caux C, et al. J Exp Med. (1994) 180: 1263-1272; Jensen S S and Gad M. J Inflamm (Lond) (2010) 7: 37; Tan J K H, O'Neill H C. J Leukocyte Biol. (2005) 78: 319-324; Iwasaki A, Medzhitov R. Nat Immunol. (2015) 16: 343-353]. Thus, the interaction between mature DCs and antigen-specific T cells is the trigger of antigen-specific immune responses [Id., citing Luft T., Blood (2006) 107: 4763-4769, Jonuleit H. Arch Dermatol Res. (1996) 289: 1-8]. When interacting with CD4+ T cells, DCs may induce their differentiation into different T helper (T_{H}) subsets [Id., citing Iwasaki A, Medzhitov R. Nat Immunol. (2015) 16: 343-353] such as T_{H}1 [Amsen D, et al. Cell (2004) 117: 515-526; Constant S, et al. J Exp Med (1995) 182: 1591-1596; Hosken N A, et al. J Exp Med. (1995) 182: 1579-1584; Kadowaki N. Allergol Int. (2007) 56: 193-199; Maekawa Y, et al. Immunity (2003) 19: 549-559; Pulendran B, et al. Proc Natl Acad Sci USA. (1999) 96: 1036-1041, T_{H}2 [Id., citing Constant S, et al. J Exp Med (1995) 182: 1591-6, Hosken N A, et al. J Exp Med. (1995) 182: 1579-1584, Jenkins S J, P. et al. J Immunol. (2007) 179: 3515-3523, Soumelis V, et al. Nat Immunol. (2002) 3: 673-680], T_{H}17 [Id., citing Bailey S L, Nat Immunol. (2007) 8: 172-180; Iezzi G, et al. Proc Natl Acad. Sci USA. (2009) 106: 876-881; Huang G, et al. Cell Mol Immunol. (2012) 9: 287-295], or other CD4+ T cell subtypes [Id., citing Levings M K, et al. Blood (2005) 105: 1162-1169]. T cell differentiation in each subtype is a complex phenomenon, that can be influenced by the cytokines in the DC tissue of origin [Id., citing Rescigno M. Dendritic cell-epithelial cell crosstalk in the gut. Immunol Rev. (2014) 260: 118-128], their maturation state [Id., citing Reis e Sousa C. Nature Rev Immunol. (2006) 6: 476-483] and cause of tissue imbalance [Id., citing Vega-Ramos J, et al. Curr Opin. Pharmacol. (2014) 17: 64-70]. DCs present a unique characteristic: the ability to perform cross-presentation [Id., citing Coulon P-G, et al. J. Immunol. (2016) 197: 517-532; Delamarre L. and Mellman I. Semin Immunol. (2011) 23: 2-11; Jung S, et al. Immunity (2002) 17: 211-220; Segura E. and Amigorena S. Adv Immunol. (2015) 127: 1-31; Segura E. and Villadangos J A. Curr Opin Immunol. (2009) 21: 105-110], defined as the presentation, in the context of class I MHC molecules (MHC-I), of antigens captured from the extracellular milieu. This feature allows DCs to trigger responses against intracellular antigens from other cell types, thus providing means for the system to deal with threats that avoid professional APC [Id., citing Coulon P-G, et al. J Immunol. (2016) 197: 517-32, Bevan M J. Cross-priming for a secondary cytotoxic response to minor H antigens with H-2 congenic cells which do not cross-react in the cytotoxic assay. J Exp Med. (1976) 143: 1283-1288, Sánchez-Paulete A R, et al. Ann Oncol. (2017) 28: xii74. doi: 10.1093/annonc/mdx727] and, even, to prime CD8+ lymphocytes in the absence of CD4+ T cells [Id., citing McCoy K D, et al. J Exp Med. (1999) 189: 1157-1162, Young J W, Steinman R M. J Exp Med. (1990) 171: 1315-1332]. Cross-presentation is involved also in the induction of tolerance to intracellular self-antigens that are not expressed by APC and then, called, cross-tolerance [Kurts C, et al. J Exp Med. (1997) 186: 239-45, Rock K L, Shen L. Immunol Rev. (2005) 207: 166-183].

Before receiving maturation stimuli, DC are said to be in an “immature state.” Immature DCs are poor inducers of naïve lymphocyte effector responses, since they have low surface expression of co-stimulatory molecules, low expression of chemokine receptors, and do not release immunostimulatory cytokines [Id., citing Trombetta E S and Mellman I. Annu Rev Immunol. (2005) 23: 975-1028, Steinman R M and Swanson J. J Exp Med. (1995) 182: 283-288]. These “immature” cells, though, are very efficient in antigen capture due to their high endocytic capacity, via receptor-mediated endocytosis, including lectin-[Id., citing Geijtenbeek T B, et al. Cell (2000) 100: 575-585; Sallusto F, et al. J Exp Med. (1995) 182: 389-400; Valladeau J, et al. Cell Immunol. (1994) 159: 323-330; Medzhitov R, et al. Nature (1997) 388: 394-397; Muzio M, et al. J Immunol. (2000) 164: 5998-6004], FC- and complement receptors [Id., citing Muzio M, et al. J Immunol. (2000) 164: 5998-6004) and macropinocytosis (Id., citing Sallusto F, et al. J Exp Med. (1995) 182: 389-400). Thus, immature DCs act not only as sentinels against invading pathogens [Id., citing Worbs T, et al. Nat Rev Immunol. (2017) 17: 30-48, Wilson N S, et al. Blood (2004) 103: 2187-2195], but also as tissue scavengers, capturing apoptotic and necrotic cells [Id., citing Albert M L, et al. Nature (1998) 392: 86-89].

This latter feature confers to immature DCs an essential role in the induction and maintenance of immune tolerance [Id., citing Steinman R M, et al. Ann NY Acad Sci. (2003) 987: 15-25; Castellano G, et al. Mol Immunol. (2004) 41: 133-140; Deluce-Kakwata-Nkor N, et al. Transfus. Clin. Biol. (2018) 25: 90-95; Liu J. and Cao X. J Autoimmun. (2015) 63: 1-12; Shiokawa A, et al. Immunology (2017) 152: 52-64]. Apoptotic cells that arise in consequence of natural tissue turnover [Id., citing Huang F P, et al. J Exp Med. (2000) 191: 435-444, Steinman R M, et al. J Exp Med. (2000) 191: 411-416] are internalized by DCs but do not induce their maturation [Id., citing Steinman R M, et al. Ann NY Acad Sci. (2003) 987: 15-25, Liu K, et al. J Exp Med. (2002) 196: 1091-1097; Stuart L M, et al. J Immunol. (2002) 168: 1627-35; Wallet M A, et al. J Exper Med. (2008) 205: 219-32]. Thus, their antigens are presented to T cells without the activating co-stimulatory signals that a mature DC would deliver, resulting in T cell apoptosis [Id., citing Kurts C, et al. J Exp Med. (1997) 186: 239-245, Hong J, et al. Chin Med J. (2013) 126: 2139-2144], anergy [Id., citing Manicassamy S. and Pulendran B. Immunol Rev. (2011) 241: 206-227, Zhu H-C, et al. Cell Immunol. (2012) 274: 12-18] or development into Tregs [Id., citing Saito M, et al. J Exper Med. (2011) 208: 235-249, Sela U, et al., PLOS ONE (2016) 11: e0146412].

These “tolerogenic DC” express less co-stimulatory molecules and proinflammatory cytokines but upregulate the expression of inhibitory molecules (like PD-L1 and CTLA-4), secrete anti-inflammatory cytokines (IL-10, for example) [Id., citing Manicassamy S. and Pulendran, B. Immunol Rev. (2011) 241: 206-227, Grohmann, U, et al. Nat Immunol. (2002) 3: 1097-1101; Morelli A E, and Thomson, A W. Nature Rev Immunol. (2007) 7: 610-621; Sakaguchi S., et al. Nat Rev Immunol. (2010) 10: 490-500] and are essential to prevent responses against healthy tissues [Id., citing Hawiger D. J Exp Med. (2001) 194: 769-779, Steinman R M, et al. Ann NY Acad Sci. (2003) 987: 15-25, Idoyaga J, et al. J Clin Invest. (2013) 123: 844-854; Mahnke K, et al. Blood (2003) 101: 4862-4869; Yates S F, et al. J Immunol (2007) 179: 967-976; Yogev N, et al. Immunity (2012) 37: 264-275].

However, in some contexts, immature DC can be harmful to the body. It is known that DC that are unable to induce lymphocyte effector responses may contribute to the immune system's failure to fight infections [Id., citing Campanelli A P, et al. J Infect Dis. (2006) 193: 1313-1322, Montagnoli C, et al. J Immunol. (2002) 169: 6298-6308] or tumors [Id., citing Baleeiro R B, et al. Cancer Immunol Immunother (2008) 57: 1335-1345; Almand B, et al. Clin Cancer Res. (2000) 6: 1755-1766; Bella S D, et al. Br J Cancer (2003) 89: 1463-1472; Dunn G P, et al. Immunity (2004) 21: 137-148; Johnson D J, Ohashi P S. Anna NY Acad Sci. (2013) 1284: 46-51; Vicari A P, et al. Semin Cancer Biol. (2002) 12: 33-42]. In these situations, DC, even after recognition of pathogens or other changes in microenvironment, fail to increase the co-stimulatory molecules required to activate T cells, thus allowing the disease to “escape” immune control.

The term “derived from” as used herein, is meant to encompasses any method for receiving, obtaining, or modifying something from a source of origin.

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like.

The term “detectable response” as used herein, is meant to refer to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “differentiate” and its various grammatical forms as used herein, are meant to refer to the process of development with an increase in the level of organization or complexity of a cell or tissue, accompanied with a more specialized function.

The term “direct presentation” as used herein refers to the process by which proteins produced within a given cell give rise to peptides presented by MHC class I molecules. This may refer to APCs (such as dendritic cells), or to nonimmune cells that will become the targets of CTLs.

The terms “disease progression” or “progressive disease” as used herein refers to a cancer that continues to grow or spread.

The term “effector cell” as used herein refers to a cell that carries out a final response or function. The main effector cells of the immune system, for example, are activated lymphocytes and phagocytes.

The term “effector functions” as used herein refers to the actions taken by effector cells and antibodies to eliminate foreign entities, and includes, without limitation, cytokine secretion, cytotoxicity, and antibody-mediated clearance.

The term “epitope” or “antigenic determinant” as used herein refers to a site on an antigen recognized by an antibody or an antigen receptor. The most common T-cell epitope is a short peptide bound to MHC molecules. B cell epitopes are typically structural motifs on the surface of the antigen.

As used herein, the term “expression” is meant to encompass production of an observable phenotype by a gene, usually b directing the synthesis of a protein. It includes the biosynthesis of mRNA, polypeptide biosynthesis, polypeptide activation, e.g., by post-translational modification, or an activation of expression by changing the subcellular location or by recruitment to chromatin.

The term “expand” or “amplify” as used herein with respect to cells refers to being grown in culture to increase cell number.

As used herein the term “Fc-gamma receptors (FcγRs)” refers to receptors that recognize IgG-coated targets, such as opsonized pathogens or immune complexes (ICs). Cross-linking leads to internalization of the cargo with associated activation of down-stream signaling cascades. FcγRs vary in their affinity for IgG and intracellular trafficking and therefore have an opportunity to regulate antigen presentation by controlling the shuttling and processing of their cargos. FcγRs bind to the IgG molecule through its Fc (fragment, crystallizable) portion [Junker, F. et al. Front. Immunol. (2020) doi.org/10.3389/fimmu.2020.01393, citing Ravetch, JV, Bolland, S. Annu. Rev. Immunol. (2001) 19: 275-290]. In humans, three groups of FcγRs have been described across a variety of cell types: FcγRI, FcγRIIA/B, FcγRIIIA/B [Id., citing Nimmerjahn, F., Ravetch, JV. Nat. Rev. Immunol. (20008) 8: 34-47]. These are expressed in differing combinations at the surface membrane of the various immune cells [Id., citing Bruhns, P. Blood (2010) 119: 5640-5649]. In the case of FcγRI, these include macrophages, neutrophils, eosinophils and DCs. For FcγRIIA, cell types include macrophages, neutrophils, eosinophils, platelets, and Langerhans cells as well as conventional, but not plasmacytoid, DCs [Id., citing Boruchov, A M et al. J. Clin. Invest. (2005) 115: 2914-23]. FcγRIIIA is found on natural killer (NK) cells and macrophages, as reviewed elsewhere [Id., citing Hayes, J M et al. J. Inflamm. Res. (2016) 9: 2009-2019]. The inhibitory Fc gamma receptor FcγRIIB is found on B cells, mast cells as well as macrophages, neutrophils, and eosinophils. Importantly, it is also expressed on cDCs [Id., citing Boruchov, A M et al. J. Clin. Invest. (2005) 115: 2914-2923]. Flow cytometry experiments suggest that it is unlikely that human pDCs, in contrast to mouse pDCs where expression of the inhibitory receptor FcγRIIB was claimed [Id., citing Flores, M. et al. J. Immunol. (2009) 183: 129-139], express any FcγRs [Id., citing Boruchov, A M et al. J. Clin. Invest. (2005) 115: 2914-2923; Patel, KR Front. Immunol. (2019) 10: 223]. Moreover, some studies on pDCs may have included contaminating cDC [Id., citing Balan, S. Int. Rev. Cell Mol. Biol. (2019) 348: 1-68]. FcγRIIIB, which can be considered a decoy receptor since it lacks association with downstream signaling molecules (as discussed at later stages of this article), is mainly expressed on neutrophils but may under certain conditions also be expressed on other immune cells like basophils [Id., citing Ravetch, J V. and Bolland, S. Annu. Rev. Immunol. (2001) 19: 275-290; Bruhns, P. Blood (2012) 119: 5640-5649].

The term “gamma: delta T cell” or “γ: δ T cell” as used herein refers to a subset of T lymphocytes bearing a T-cell receptor composed of the antigen recognition chains γ and δ, assembled in a γ: δ heterodimer (meaning a dimer of one γ chain and one δ chain that makes up the antigen-recognition portion of a γ: δ T-cell receptor. γ: δ T-cell receptors are antigen receptors composed of a γ and a δ chain carried by a subset of T lymphocytes that is distinct from the αβ T cell receptor.

The term “germinal center” as used herein refers to sites of intense B-cell proliferation and differentiation that develop in lymphoid follicles during an adaptive immune response. Somatic hypermutation and class switching occur in germinal centers.

The term “helper T cells” or “TH” cells as used herein refers to effector CD4 T cells that stimulate or “help” B cells to make antibody in response to antigenic challenge. TH2, TH1 and the THF subsets of effector CD4 T cells can perform this function.

As used herein, the term “immune checkpoints” refers to the array of inhibitory pathways necessary for maintaining self-tolerance and that modulate the duration and extent of immune responses to minimize damage to normal tissue. Immune checkpoint molecules such as PD-1, PD-L1, CTLA-4 are cell surface signaling receptors that play a role in modulating the T-cell response in the tumor microenvironment. Tumor cells have been shown to utilize these checkpoints to their benefit by up-regulating their expression and activity. With the tumor cell's ability to commandeer some immune checkpoint pathways as a mechanism of immune resistance, it has been hypothesized that checkpoint inhibitors that bind to molecules of immune cells to activate or inactivate them may relieve the inhibition of an immune response. Immune checkpoint inhibitors have been reported to block discrete checkpoints in an active host immune response allowing an endogenous anti-cancer immune response to be sustained. Recent discoveries have identified immune checkpoints or targets, like PD-1, PD-L1, PD-L2, CTLA4, TIGIT, TIM-3, LAG-3, CCR4, OX40, OX40L, IDO, and A2AR, as proteins responsible for immune evasion.

The terms “immune escape” or “immune evasion” as used herein refers to a strategy to evade a host's immune response. It is characterized by the inability of the immune system to eliminate transformed cells prior to and after tumor development. The host's contribution is manifested by the inability to recognize antigens expressed by tumor cells, a phenomenon known as “host ignorance.” It happens because of defects in both the innate and adaptive arms of the immune system. The tumor's contribution is manifested by the adaptation of tumor cells to evade the immune systems or by developing a microenvironment that suppresses the immune system. [Qian J. et al. (2011) Immune Escape. In: Schwab M. (eds) Encyclopedia of Cancer. Springer, Berlin, Heidelberg. https://doi.org/10.1007/978-3-642-16483-5_2975].

The term “immune homeostasis” refers to the delicate and finely regulated balance of appropriate immune activation and suppression in tissues and organs, driven by a myriad of cellular players and chemical factors. [da Gama Duarte, J. et al. Immnology and Cell Biology (2018) 96: 497-506]

The term “immune phenotype” or “immunotype” as used herein refers to the collective frequency of various immune cell populations and their functional responses to stimuli (cell signaling and antibody responses) [See Kaczorowski, K J et al. Proc. Nat. Acad. Sci. USA (2017) doi/10.1073/pnas.1705065114].

The terms “immune response” and “immune-mediated” are used interchangeably herein to refer to any functional expression of a subject's immune system, against either foreign or self-antigens, whether the consequences of these reactions are beneficial or harmful to the subject. The term “immunological response” to an antigen or composition as used herein, is meant to refer to the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves an antigen-specific response by helper T-cells. Helper T-cells act to help stimulate the function and focus the activity of nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γ δ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The terms “immune surveillance” or “immunological surveillance” are used interchangeably to refer to a monitoring process by the immune system to detect and destroy virally infected and neoplastically transformed cells in the body.

The term “immune system” as used herein refers to the body's system of defenses against disease, which comprises the innate immune system and the adaptive immune system. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

The term “immunological repertoire” refers to the collection of transmembrane antigen-receptor proteins located on the surface of T and B cells. [Benichou, J. et al. Immunology (2011) 135: 183-191]. The combinatorial mechanism that is responsible for encoding the receptors does so by reshuffling the genetic code, with a potential to generate more than 1018 different T cell receptors (TCRs) in humans [Id., citing Venturi, Y. et al. Nat. Rev. Immunol. (2008) 8: 231-238] and a much more diverse B-cell repertoire. These sequences, in turn, will be transcribed and then translated into protein to be presented on the cell surface. The recombination process that rearranges the gene segments for the construction of the receptors is key to the development of the immune response, and the correct formation of the rearranged receptors is critical to their future binding affinity to antigen. For example, diversity of the TCR gene is generated by rearrangement of the V and J gene segments during T cell development in the thymus. (Makino, Y., et al (1993) J. Exptl Med. 177: 1399-1408). The TCR V and J gene segments, like Ig genes, possess recombination signals in which heptamer and nonamer sequences, separated by a 12/23 bp spacer, are flanked by germline V and J gene segments [Id.].

The term “immunogen” and its various grammatical forms as used herein is used interchangeably with the term “antigen”.

Immunological synapses and immune cell activation. Immune responses are initiated by the interaction between antigen presenting cells (APCs), such as dendritic cells (DCs), with responder cells, such as T cells, via a tight cellular contact interface called the immunological synapse. The immunological synapse is a highly organized subcellular structure that provides a platform for the presentation of antigen in major histocompatibility class I and II complexes (MHC class I and II) on the surface of the APC to receptors on the surface of the responder cells. In T cells, these contacts lead to highly polarized membrane trafficking that results in the local release of lytic granules and in the delivery and recycling of T cell receptors at the immunological synapse. Localized trafficking also occurs at the APC side of the synapse, especially in DCs where antigen loaded in MHC class I and II is presented and cytokines are released specifically at the synapse. A functional immunological synapse between DCs and naïve T cells is essential to mount functional T cell responses. [Vergoogen, D R J et al. Biomol Concepts (2016) 7 (1): 17-28].

Not only DCs and T cells, but also other APCs, such as B cells or infected cells, and other effector cells, such as natural killer cells (NKs), form immunological synapses for intercellular communication as well as for the killing of infected or aberrant target cells. [Id., citing Angus, K L and Griffiths, GM. Curr. Opin. Cell Biol. (2013) 25: 85-91; Friedl, P. et al. Nat. Rev. Immunol. (2005) 5: 53; Xie, J. et al. Immunol. Rev. (2013) 251: 65-79]. The structure of the synapse strongly depends on the cell types involved, the presence and strength of antigen recognition and additional co-stimulatory interactions. [Id., citing Friedl, P. et al. Nat. Rev. Immunol. (2005) 5: 53; Thauland, T J and Parker, DC. Immunology (2010) 131: 466-472; Azar, G A et al. Proc. Natl. Acad. Sci. USA (2010) 107: 3675-3680].

Immunological synapses can functionally be divided into two categories [Id., citing Gerard, A. et al. Immunol. Rev. (2013) 251: 80-96]: (1) primary synapses, which are the cell-cell contacts that result in initial activation of immune cells, such as the synapses between DCs and T cells [Id., citing Rodriguez-Fernandez, J L et al. Sci. Signal (2010) 3: re2], and (2) so-called secondary synapses that result from interactions established after initial priming, such as activated T cells delivering stimulatory signals via, for example, CD40-CD40L interactions to B cells [Id., citing Chaplin, DD. J. Allergy Clin. Immunol. (2010) 125: S3-23]; this category also encompasses the synapses formed between NKs or cytotoxic T cells with their target APC where lytic granules are released to kill the APC [Id., citing Stinchcombe, J C et al. Immunity (2001) 15: 751-61]. For both categories, the formation of immunological synapses can trigger intracellular signaling cascades in both the APC and the T cell that lead to reorganization of the cytoskeleton and rerouting of membrane trafficking.

Membrane trafficking. Membrane trafficking plays an important role in T cell effector functions, because it leads to surface display of TCRs and other membrane proteins, recycling of exhausted receptors, and to release of cytokines and chemokines at the immunological synapse. The best understood form of exocytosis at the immunological synapse is the release of cytolytic granules from CD8+ T cells and NKs. Other types of cargo that are delivered and/or recycled at the T cell side of the immunological synapse include cytokines (e.g. IFN-gamma), and membrane receptors (e.g. TCR, ICAM-1) [Id., citing Griffiths, G M et al. J. Cell Biol. (2010) 189: 397-406; Angus, K L. and Griffiths, GM. Curr. Opin. Cell Biol. (2013) 25: 85-91; Xie, J. et al. Immunol. Rev. (2013) 251: 65-79; Jo, J H. et al. J. Cell Biochem. (2010) 111: 1125-37; Finetti, F. and Baldari, CT. Immunol. Rev. (2013) 251: 97-112; Das, V. et al. Immunity (2004) 20: 577-88; Soares H. et al. J. Exp. Med. (2013) 210: 2415-2433]. The polarized delivery of these molecules to the immunological synapse allows a more sensitive antigen presentation and/or promotes T cell effector functions, while preventing unwanted activation of other (immune) cells nearby.

Polarized membrane trafficking occurs at the APC side as well. In DCs, MHC class I and II [Id., citing Boes, M. et al. Nature (2002) 418: 983-988; Bertho, N. J. Immunol. (2003) 171: 5689-5696; Boes, M. et al. J. Immunol. (2003) 171: 4081-4088; Compeer, E B et al. J. Biol. Chem. (2014) 289: 520-528] and the costimulatory molecule CD40 [Id., citing Foster, N. et al. J. Immunol. (2012) 189: 5632-5637] can be locally trafficked to and presented at the immunological synapse. The local release of these molecules improves the efficiency of T cell activation and helps to explain how T cells can detect a few MHC ligands among an abundance of endogenous peptide-bound MHC [Id., citing Xie, J. et al. Immunol. Rev. (2013) 251: 65-79]. In addition, IL-12 is also locally released by the DC at the immunological synapse with T cells [Id., citing Pulecio, J. et al. J. Exp. Med. (2010) 207: 2719-2732]. IL-12 promotes a T_{H}1 response, enhances the cytolytic activity of CD8+ T cells and induces production of IFN-gamma by T cells. The polarized release of IL-12 also was observed at the immunological synapse between DCs and NKs [Id., citing Borg, C. et al. Blood (2004) 104: 3267-3275; Barreira da Silva, R. et al. Blood (2): 6487-6498].

The terms “immunomodulatory”, “immune modulator”, “immunomodulatory,” and “immune modulatory” are used interchangeably herein to refer to a substance, agent, or cell that is capable of augmenting or diminishing immune responses directly or indirectly, e.g., by expressing chemokines, cytokines and other mediators of immune responses.

As used herein, the term “immunostimulatory” and its other grammatical forms refers to augmenting an immune response either directly or indirectly.

As used herein, the term “immunosuppressive” and its other grammatical forms refers to suppressing or diminishing an immune response either directly or indirectly.

As used herein, the term “immunostimulatory amount” refers to an amount of an immunogenic composition that stimulates an immune response by a measurable amount, for example, as measured by ELISPOT assay (cellular immune response), ICS (intracellular cytokine staining assay) and major histocompatibility complex (MHC) tetramer assay.

As used herein the term “immunosuppressive amount” refers to an amount of an immunosuppressive composition that suppresses an immune response, for example, as measured by ELISPOT assay (cellular immune response), ICS (intracellular cytokine staining assay) and major histocompatibility complex (MHC) tetramer assay.

The term “immunotherapy” as used herein refers to the measures taken using immunological methods and principles to target the hyper or hyo-immune state of an organism, intervene or adjust the organism's immune function artificially, and strengthen or attenuate the immune response so as to treat disease. It enhances the immune system's ability to recognize, target and eliminate cancer cells in the body. [Zhang, Z. et al. Front. Immunol. (2021) 12: 72356; Barbari, C. et al. Int'l J. Mol. Sci. (2020) 21: 5009]. Some types of immunotherapy only target certain cells of the immune system. Others affect the immune system in a general way. For example, monoclonal antibodies can attach to specific proteins on the surface of cancer cells or immune cells in order to mark the cancer as a target for the immune system or boost the ability of immune cells to fight the cancer. Cytokine therapy, another example, relies on proteins called interferons and interleukins to trigger an immune response. Interleukin-2 (IL-2) is used to treat kidney cancers and melanomas that have spread to other regions of the body. Interferon alpha (IFN-alpha) is currently being used to treat melanoma, kidney cancer and certain leukemias and lymphomas. These cytokine treatments are also being combined with other types of immunotherapies to increase their effectiveness.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “inflammatory mediators” or “inflammatory cytokines” as used herein refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, and proinflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12). Among the pro-inflammatory mediators, IL-1, IL-6, and TNF-α are known to activate hepatocytes in an acute phase response to synthesize acute-phase proteins that activate complement.

The term “inhibitor receptor lymphocyte activation gene-3” or “LAG-3” as used herein refers to a member of the immunoglobulin superfamily (IgSF) and binds to major histocompatibility complex (MHC) class II. LAG-3 expression on TILs is associated with tumor-mediated immune suppression.

The term “innate immunity” as used herein refers to the various mechanisms encountered by a pathogen or transformed cell before adaptive immunity is induced, such as anatomical barriers, antimicrobial peptides, the complement system, and macrophages and neutrophils carrying nonspecific pattern-recognition receptors. Innate immunity is present in all individuals at all times, does not increase with repeated exposure, and discriminates between groups of similar pathogens, rather than responding to a particular pathogen.

The term “interleukin (IL)” as used herein refers to a cytokine secreted by, and acting on, leukocytes. Interleukins regulate cell growth, differentiation, and motility, and stimulates immune responses, such as inflammation. Examples of interleukins include, without limitation, interleukin-1 (IL-1), interleukin-1β (IL-1β), interleukin-2 (IL-2), interleukin-6 (IL-6), interleukin-7 (IL-7), interleukin-8 (IL-8), interleukin-12 (IL-12), interleukin-15 (IL-15), and interleukin 37 (IL-37).

The term “isolated” is used herein to refer to material, such as, but not limited to, a nucleic acid, peptide, polypeptide, protein, or cell which is: (1) substantially or essentially free from components that normally accompany or interact with it as found in its naturally occurring environment. For example, a naturally occurring polynucleotide or polypeptide present in a living animal is not isolated, but the same polynucleotide or polypeptide, separated from some or all of the coexisting materials in the natural system, is isolated. The terms “substantially free” or “essentially free” are used herein to refer to considerably or significantly free of, or more than about 95% free of, or more than about 99% free of such components. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment.

The term “labeling” as used herein refers to a process of distinguishing a compound, structure, protein, peptide, antibody, cell or cell component by introducing a traceable constituent. Common traceable constituents include, but are not limited to, a fluorescent antibody, a fluorophore, a dye or a fluorescent dye, a stain or a fluorescent stain, a marker, a fluorescent marker, a chemical stain, a differential stain, a differential label, and a radioisotope.

The term “leukapheresis” as used herein refers to a laboratory procedure in which blood passes through a machine that removes the white blood cells and returns all other blood cells and plasma back into the bloodstream.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. Two broad classes of lymphocytes are recognized: the B-lymphocytes (B-cells), which are precursors of antibody-secreting cells, and T-lymphocytes (T-cells),

The term “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. For example, a mature B cell can be activated by an encounter with an antigen that expresses epitopes that are recognized by its cell surface immunoglobulin Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC). The soluble product of an activated B lymphocyte is immunoglobulins (antibodies). The soluble product of an activated T lymphocyte is lymphokines (meaning cytokines produced by lymphocytes).

The term “machine learning” as used herein refers to a subset of artificial intelligence and computer science that uses algorithms that learn from data to make predictions. These predictions can be generated through supervised learning, where algorithms learn patterns from existing data, or unsupervised learning, where they discover general patterns in data.

The term “macrophage” as used herein refers to a mononuclear, actively phagocytic cell arising from monocyte stem cells in the bone marrow. These cells are widely distributed in the body and vary in morphology and motility. Phagocytic activity is typically mediated by serum recognition factors, including certain immunoglobulins and components of the complement system, but also may be nonspecific. Macrophages also are involved in both the production of antibodies and in cell-mediated immune responses, particularly in presenting antigens to lymphocytes. They secrete a variety of immunoregulatory molecules. Macrophages have been classified based on their mode of activation: classically activated/M1 macrophages respond to interferon-gamma (IFN-γ) by releasing pro-inflammatory cytokines and are involved in TH1 cell mediated resolution of acute infection. Alternatively activated/M2 macrophages respond to cytokines from TH2 cells and are involved in wounding and fibrosis. [Ghajar, C M et al., “The role of the microenvironment in tumor initiation, progression, and metastasis,” In Mendelsohn, J. et al., the Molecular Basis of Cancer, Elsevier Saunders, Philadelphia, citing Pollard, J W, Nat. Rev. Immunol. (2009) 9: 259-270]. The diverse functions of macrophages are executed in a tissue- and context-specific fashion by a number of discrete macrophage subtypes, which aid these developmental processes by remodeling collagen and secreting a host of pro-angiogenic, pro-inflammatory and matrix-degrading factors (Id., citing Qian, B Z. and Pollard, J W. Cell (2010) 141: 39-51).

The terms “Major Histocompatibility Complex (MHC), MHC-like molecule” and “HLA” are used interchangeably herein to refer to cell-surface molecules that display a molecular fraction known as an epitope or an antigen and mediate interactions of leukocytes with other leukocyte or body cells. MHCs are encoded by a large gene group and can be organized into three subgroups-class I, class II, and class III. In humans, the MHC gene complex is called HLA (“Human leukocyte antigen”); in mice, it is called H-2 (for “histocompatibility”). Both species have three main MHC class I genes, which are called HLA-A, HLA-B, and HLA-C in humans, and H2-K, H2-D and H2-L in the mouse. These encode the α chain of the respective MHC class I proteins. The other subunit of an MHC class I molecule is β2-microglobulin. The class II region includes the genes for the α and β chains (designated A and B) of the MHC class II molecules HLA-DR, HLA-DP, and HLA-DQ in humans. Also in the MHC class II region are the genes for the TAP1: TAP2 peptide transporter, the PSMB (or LMP) genes that encode proteasome subunits, the genes encoding the DMα and BMβ chains (DMA and DMB), the genes encoding the α and β chains of the DO molecule (DOA and DOB, respectively), and the gene encoding tapasin (TAPBP). The class II genes encode various other proteins with functions in immunity. The DMA and DMB genes encoding the subunits of the HLA-DM molecule that catalyzes peptide binding to MHC class II molecules are related to the MHC class II genes, as are the DOA and DOB genes that encode the subunits of the regulatory HLA-DO molecule. [Janeway's Immunobiology. 9th ed., GS, Garland Science, Taylor & Francis Group, 2017. pps. 232-233]. In humans, there are three MHC class II isotypes: HLA-DR, HLA-DP, and HLA-DQ, encoded by α and β chain genes within the Human Leukocyte Antigen (HLA) locus on chromosome 6 [Wosen, J E et al. Front. Immunol. (2018) doi.10.3389/fimmu.2018.02144].

The term “MHC restriction” as used herein refers to the requirement that APCs or target cells express MHC molecules that a T cell recognizes as self in order for T cell to respond to the antigen presented by that APC or target cell (T cells will only recognize antigens presented by their own MHC molecules). For example, CD8 T cells bind class I MHC which are expressed on most cells in the body, and CD4 T cells bind class II MHC which are only expressed on specialized APCs.

MHC tetramers are used for the detection of antigen-specific T cell populations.

The term “mediated” and its various grammatical forms as used herein refers to depending on, acting by or connected through some intervening agency.

The term “memory cells” as used herein refers to B and T lymphocytes generated during a primary immune response that remain in a quiescent state until fully activated by a subsequent exposure to specific antigen (secondary immune response). Memory cells generally are more sensitive than naïve lymphocytes to antigen and respond rapidly on re-exposure to the antigen that originally induced them. During an immune response, naïve T cells (T_{N}) are primed by antigen-presenting cells (APCs). Depending on the strength and quality of stimulatory signals, proliferating T cells progress along a differentiation pathway that culminates in the generation of terminally differentiated short-lived effector T (T_{EFF}) cells. When antigenic and inflammatory stimuli cease, primed T cells become quiescent and enter into the memory stem cell (T_{SCM}), central memory (T_{CM}) cell or effector memory (T_{EM}) cell pools, depending on the signal strength received. T_{SCM} cells possess stem cell-like attributes to a greater extent than any other memory lymphocyte population. Although both T_{CM} and T_{EM} cells can also undergo self-renewal, the capacity to form diverse progeny is progressively restricted, so that only T_{SCM} cells are capable of generating all three memory subsets and T_{EFF} cells; T_{CM} cells can give rise to T_{CM}, T_{EM} and T_{EFF} cells, and T_{EM} cells can only produce themselves and T_{EFF} cells. [Gattinoni, L. et al. Nature Revs. Cancer 12 (2012) 671-684].

The term “metastasis” as used herein refers to spread of cancer cells from the place where they first formed to another part of the body. In metastasis, cancer cells break away from the original (primary) tumor, travel through the blood or lymph system, and form a new tumor in other organs or tissues of the body. The new, metastatic tumor is the same type of cancer as the primary tumor. For example, if breast cancer spreads to the lung, the cancer cells in the lung are breast cancer cells, not lung cancer cells.

The term “myeloid” as used herein means of or pertaining to bone marrow.

Granulocytes and monocytes, collectively called myeloid cells, are differentiated descendants from common progenitors derived from hematopoietic stem cells in the bone marrow. Commitment to either lineage of myeloid cells is controlled by distinct transcription factors followed by terminal differentiation in response to specific colony-stimulating factors and release into the circulation. Upon pathogen invasion, myeloid cells are rapidly recruited into local tissues via various chemokine receptors, where they are activated for phagocytosis as well as secretion of inflammatory cytokines, thereby playing major roles in innate immunity. [Kawamoto, H. and Minato, N. Int'l J. Biochem. Cell Biol. (2004) 36 (8): 1374-1379].

The term “myeloid-derived suppressor cells” as used herein refers to a heterogeneous population of cells that represent a pathologic state of activation of monocytes and relatively immature neutrophils. MDSCs are characterized by a distinct set of genomic and biochemical features, and can, on the basis of recent findings, be distinguished by specific surface molecules. The salient feature of these cells is their ability to inhibit T cell function and thus contribute to the pathogenesis of various diseases. [Veglia, F. et al. Nature Immunol. (2018) 19: 108-119].

The term “naïve T cell” as used herein refers to a T cell that has not previously been exposed to an antigen. Naïve T cells are conventionally defined by coexpression of the RA isoform of the transmembrane phosphatase CD45, the lymph node homing molecules L-selectin (CD62L) and CCR7, and the costimulatory receptors CD27 and CD28. [De Rosa, S C et al. Nature Med. (2001) 7: 245-248].

The term “natural killer (NK) cells” as used herein is meant to refer to lymphocytes in the same family as T and B cells, classified as group I innate lymphocytes. They have an ability to kill tumor cells without any priming or prior activation, in contrast to cytotoxic T cells, which need priming by antigen presenting cells. NK cells secrete cytokines such as IFNγ and TNFα, which act on other immune cells, like macrophages and dendritic cells, to enhance the immune response. Activating receptors on the NK cell surface recognize molecules expressed on the surface of cancer cells and infected cells and switch on the NK cell. Inhibitory receptors act as a check on NK cell killing. Most normal healthy cells express MHCI receptors, which mark them as “self.” Inhibitory receptors on the surface of the NK cell recognize cognate MHCI, which switches off the NK cell, preventing it from killing. Once the decision is made to kill, the NK cell releases cytotoxic granules containing perforin and granzymes, which leads to lysis of the target cell. Natural killer reactivity, including cytokine secretion and cytotoxicity, is controlled by a balance of several germline encoded inhibitory and activating receptors such as killer immunoglobulin-like receptors (KIRs) and natural cytotoxicity receptors (NCRs). The presence of the MHC Class I molecule on target cells serves as one such inhibitory ligand for MHC Class I-specific receptors, the Killer cell Immunoglobulin-like Receptor (KIR), on NK cells.

Engagement of KIR receptors blocks NK activation and, paradoxically, preserves their ability to respond to successive encounters by triggering inactivating signals. Therefore, if a KIR is able to sufficiently bind to MHC Class I, this engagement may override the signal for killing and allows the target cell to live. In contrast, if the NK cell is unable to sufficiently bind to MHC Class I on the target cell, killing of the target cell may proceed. Consequently, those tumors which express low MHC Class I and which are thought to be capable of evading a T-cell-mediated attack may be susceptible to an NK cell-mediated immune response instead.

The term “NKG2D” as used herein refers to an activating receptor expressed by all NK cells and subsets of T cells (γδ T cells, CD8+ T cells and CD4+ T cells) in humans. It is encoded by the KLRK1 gene (killer cell lectin-like receptor subfamily K, member 1). NKG2D receptor functions as an activating receptor by virtue of its interactions with the signaling adaptor dimer DAP10 in humans and with DAP10 and DAP12 in mice (Raulet, D H et al. Annu. Rev. Immunol. (2013) 31: 4123-4141, citing Champsaur, M. and Lanier, LL. Immunol. Rev. (2010) 235: 267-285; Wu, J. et al. Science (1999) 285: 730-732). When the receptor is ligated, DAP10 provides signals that recruit the p85 subunit of phosphatidylinositol 3-kinase (PI3K) and a complex of GRB2 and VAV1. Engagement of NKG2D on NK cells induces degranulation and cytokine production.

NK cell activation as a result of NKG2D engagement can modify, or be modified by, engagement of other NK receptors. For naïve human NK cells, synergistic activation occurs when NKG2D is coengaged with 2B4, a SLAM family receptor whose ligand is broadly expressed by hematopoietic cells, or with NKp46, another activating receptor [Id., citing Bryceson, Y T et al. Blood (2006) 107: 159-166]. Conversely, NKG2D-induced NK activation can be inhibited (albeit not necessarily completely) if the target cell expresses MHC class I molecules that engage inhibitory receptors on NK cells, such as KIRs (killer cell immunoglobulin-like receptors) in humans [Id., citing Jamieson, A M et al. Immunity (2002) 17: 19-29; Rgunathan, J. et al. Blood (2005) 105: 2133-2140].

NKG2D binds to several different ligands, all of which are homologous to MHC class I molecules but have no known role in antigen presentation [Id., citing Raulet, DH. Nat. Rev. Immunol. (2003) 3: 7817-90; Champsaur, M. and Lanier, LL. Immunol. Rev. (2010) 235: 267-285; Eagle, R A and Trowsdale, J. Nat. Rev. Immunol. (2007) 7: 737-744; Machuldova, A. et al. Front. Immunol. (2021) 12: 651751, citing Stephens, HA. Trends Immunol. (2001) 22 (7): 378-385]. Like MHC proteins, they exhibit considerable allelic variation. In humans, the NKG2D ligands include MHC class I chain-related protein A (MICA) and MHC class I chain-related protein B (MICB), both encoded by genes in the MHC, and up to six different proteins called Unique long (UL) 16-binding proteins (ULBPs), also known as retinoic acid early transcript 1 (RAET1) proteins. Like MHC proteins, the NKG2D ligands exhibit considerable allelic variation.

All NKG2D ligands are encoded by distinct genes in the host's own genome, i.e., the ligands are self-proteins. NKG2D ligands are expressed poorly or not at all by most normal cells but are upregulated in cancer cells and virus-infected cells. This type of recognition process, in which self-coded ligands for activating receptors are induced on unhealthy cells, has been termed “induced self-recognition [Id. citing Diefenbach, A. and Raulet, DH. Immunol. Rev. (2001) 181: 170-84], which is distinct from “missing self-recognition,” a phenomenon in which loss of MHC ligands for NK inhibitory receptors sensitizes cells for elimination by NK cells. Various cellular pathways activated as a result of cellular stress, infection, or tumorigenesis regulate expression of the NKG2D ligands.

The structures of NKG2D-ligand complexes indicate that NKG2D binds diagonally over the α1 and α2 helices of the ligands, much as T-cell receptors bind over MHC molecules. Despite the poor homology of different ligands, some of the key residues that interact with NKG2D are conserved, and the NKG2D residues involved in binding are similar in the different structures. NKG2D ligands are generally poorly expressed by normal cells, but are upregulated in transformed, infected and, in some cases, stressed cells.

The engagement of NKG2D is a sufficient stimulus to activate cytolysis and cytokine production by NK cells. However, it provides an enhancing or co-stimulatory signal for the activation of CD8+ T cells and probably other T cells.

The term “necrosis” as used herein refers to an irreversible insult that interferes with a vital structure or function of an organelle (plasma membrane, mitochondria, etc.) of a cell and does not trigger apoptosis. Such insults include infectious agents (e.g., bacteria, viruses, fungi, parasites), oxygen deprivation or hypoxia, and extreme environmental conditions such as heat, radiation, or exposure to ultraviolet irradiation. At the cellular level, necrosis is characterized by cell and organelle swelling, ATP depletion, increased plasma membrane permeability, release of macromolecules and eventually inflammation. The processes by which cells undergo death by necrosis vary according to the cause, organ and cell type. While the best studied is ischemic necrosis of cardiac myocyte, the basic processes involved are comparable to those in other organs. Some of the unfolding events may occur simultaneously; others may be sequential. These are:
(1) interruption of blood supply decreases delivery of oxygen and glucose;
(2) anaerobic glycolysis leads to overproduction of lactate and decreased intracellular pH;
(3) distortion of the activities of ionic pumps in the plasma membrane skews the ionic balance of the cell;
(4) activation of phospholipase A2 (PLA2) and proteases disrupts the plasma membrane and cytoskeleton;
(5) the lack of oxygen impairs mitochondrial electron transport, thus decreasing ATP synthesis and facilitating production of ROS;
(6) mitochondrial damage promotes the release of cytochrome c to the cytosol;
(7) the cell dies. [Rubin's Pathology: Clinicopathologic Foundations of Medicine, 5th Ed. McDonald, J M, Michalopoulos, G K, Trojanowski, J Q, Ward, P A, Eds Lippincott Williams & Wilkins, MD (2008), pp. 23-26.]

Types of necrosis include, for example:
liquefactive necrosis (occurs when the rate of dissolution of the necrotic cell is considerably faster than the rate of repair; the polymorphonuclear leukocytes of the acute inflammatory reaction contain potent hydrolases capable of digesting dead cells. [Id.]
coagulative necrosis, which refers to light microscopic alterations in a dead or dying cell including pyknosis (the nucleus becomes smaller and stains deeply basophilic as chromatin clumping continues); karyorrhexis (meaning the pyknotic nucleus breaks up into many smaller fragments scattered about the cytoplasm); and karyolysis (referring to the extrusion of the pyknotic nucleus from the cell or progressive loss of chromatin staining). [Id.]
caseous necrosis (which is characteristic of tuberculosis and attributed to the toxic effects of the microbacterial cell wall. In casseous necrosis, the necrotic cells fail to retain their cellular outlines; the dead cells persist indefinitely as amorphous, coarsely granular, eosinophilic debris). [Id.]
fat necrosis, which specifically affects adipose tissue and most commonly results from pancreatitis or trauma; the process begins when digestive enzymes normally found only in the pancreatic duct and small intestine, are released from injured pancreatic acinar cells and ducts into the extracellular spaces. On extracellular activation, these enzymes digest the pancreas itself and surrounding tissues, including adipose cells. [Id.]
fibrinoid necrosis (which is an alteration of injured blood vessels in which insudation and accumulation of plasma proteins cause the wall to stain intensely with eosin. The eosinophila of the accumulated plasma proteins obscures the underlying alterations in the blood vessel making it difficult, if not impossible, to determine whether there truly is necrosis in the vascular wall.) [Id.]
gangrenous necrosis (used to describe ischemic necrosis of the lower limbs, sometimes upper limbs or digits). [Id.]

The term “neoantigen” as used herein refers to a new protein that forms on cancer cells when certain mutations occur in tumor DNA.

The term “neoepitope” as used herein refers to tumor-specific MHCI restricted epitopes.

The term “neutrophils” or “polymorphonuclear neutrophils (PMNs)” as used herein refers to the most abundant type of white blood cells in mammals, which form an essential part of the innate immune system. They form part of the polymorphonuclear cell family (PMNs) together with basophils and eosinophils. Neutrophils are normally found in the blood stream. During the beginning (acute) phase of inflammation, particularly as a result of bacterial infection and some cancers, neutrophils are one of the first-responders of inflammatory cells to migrate toward the site of inflammation. They migrate through the blood vessels, then through interstitial tissue, following chemical signals such as interleukin-8 (IL-8) and C5a in a process called chemotaxis, meaning the directed motion of a motile cell or part along a chemical concentration gradient toward environmental conditions it deems attractive and/or away from surroundings it finds repellent.

The term “Next Generation Sequencing” or “NGS” as used herein refers to a method of parallel sequencing. For instance, a nucleic acid (e.g., DNA) sample is obtained and prepared into a library (meaning a collection of nucleic acid fragments from the sample). The library is prepared by fragmenting the DNA or RNA sample. Fragmentation can be performed by physical (e.g., sheared by acoustics, nebulization, centrifugal force, needles, or hydrodynamics) or enzymatic (e.g., site-specific or non-specific nucleases) methods. In some embodiments, the fragments are about 200 bp, about 20 bp, about 300 bp, or about 350 bp in length. The DNA or RNA samples are repaired at the ends (e.g., blunt-ended) and then A-tailed (e.g., an adenosine is added to the 3′ end resulting in an overhang). Adapters are ligated to each end. Adapters include sequences, such as barcodes, restriction sites, and primer sequences.

The term “non-expanded” as used herein, is meant to refer to a cell population that has not been grown in culture (in vitro) to increase the number of cells in the cell population.

The term “non-replicating” or “replication-impaired” virus refers to a virus that is not capable of replication to any significant extent in the majority of normal mammalian cells or normal primary human cells.

The term “normal healthy subject” as used herein refers to a subject having no symptoms or other evidence of a cancer.

The term “PAMPs” is an abbreviation for pathogen-associated molecular patterns. PAMPS are structural patterns present in components or products common to a wide variety of microbes but not host cells. PAMPS are ligands for pattern recognition molecules (PRMs).

The term “paratrope” as used herein refers to the antigen-binding site of an antibody, which recognizes and binds to the epitope of an antigen.

The term “pattern recognition molecules” or “PRMs” as used herein refer to proteins recognizing PAMPs. Soluble PRMs include the collectins, acute phase proteins and NOD proteins. Membrane-bound PRMs are pattern recognition receptors.

The term “pattern recognition receptors” or “PRRs” refers to widely distributed membrane bound PRMs fixed in either the plasma membrane of a cell or in the membranes of its endocytic vesicles. The term PRRs includes toll-like receptors (TLRs) and scavenger receptors. Engagement of PRRs induces pro-inflammatory cytokines.

The term “peptide” is used herein to designate a series of amino acid residues, connected one to the other typically by peptide bonds between the alpha-amino and carbonyl groups of the adjacent amino acids. Peptides are typically 9 amino acids in length but can be as short as 8 amino acids in length, and as long as 14 amino acids in length. A series of amino acids are considered an “oligopeptide” when the amino acid length is greater than about 14 amino acids in length, typically up to about 30 to 40 residues in length. When the amino acid residue length exceeds 40 amino acid residues, the series of amino acid residues is termed a “polypeptide”.

The terms “peripheral blood mononuclear cells” or “PBMCs” are used interchangeably herein to refer to blood cells having a single round nucleus such as, for example, a lymphocyte or a monocyte. When a density gradient (e.g., Ficoll®) fractionation of peripheral blood method is used, PBMCs remain at the less dense, upper interface of the Ficoll® layer, often referred to as the buffy coat, and are the cells collected. These cells consist of lymphocytes (T cells, B cells, NK cells) and monocytes. In humans, lymphocytes make up the majority of the PBMC population, followed by monocytes, and only a small percentage of dendritic cells.

The term “plasma cell” as used herein refers to terminally differentiated B cells that secrete antibody. They may be short-lived, with no isotype switching or somatic hypermutation, or long lived, meaning they undergo isotype switching and somatic hypermutation.

The term “plasmablasts” as used herein refer to proliferating progeny of an activated B cell. Plasmablasts become plasma cells. Antigen binding to the BCR triggers activation of Src family kinases such as Lyn and Fyn leading to phosphorylation of Igα (CD79a) and Igβ (CD79b), recruitment of Syk kinase and subsequent recruitment and phosphorylation of BLNK, Btk and PLCγ [Luo, W. et al. J. Immunol. (2014) 193 (2): 909-920, citing Packard, T A & Cambier, J C. F1000 prime reports (2013) 5: 40]. These events activate the Ras pathway, PKC pathway and calcium flux, eventually triggering the activation of NF-κB, Erk and JNK. These positive signals are normally counterbalanced by negative signals that limit B cell activation and prevent spontaneous B cell proliferation and differentiation to plasma cells [Id., citing Nitschke, L. Curr. Opin. Immunol. (2005) 17: 2990-2997]. Negative signals are generated by a series of membrane receptors (CD22, CD72, FcγRIIb, PIR-B, Siglec-G, etc.) that are phosphorylated by Lyn. This allows them to recruit phosphatases such as SHP1 and SHIP1 that reverse phosphorylation of signaling molecules in the BCR pathway and dampen BCR signaling [Id., citing Poe, J C & Tedder, T F. Trends Immunol. (2012) 33: 413-420; Tsubata, T. Infectious disorders drug targets (2012) 12: 181-190; Vang, T. et al. Annu. Rev. Immunol. (2008) 26: 29-55].

The term “polymerase chain reaction” or “PCR” as used herein refers to a laboratory technique for rapidly producing (amplifying) millions to billions of copies of a specific segment of DNA, which can then be studied in greater detail. PCR involves using short synthetic DNA fragments called primers to select a segment of the genome to be amplified, and then multiple rounds of DNA synthesis to amplify that segment.

The term “potentiate” and its other grammatical forms as used herein means to increase the power, effect, or potency, of; to enhance, to augment the activity of.

The term “predictive biomarker” refers to a biomolecule that indicates therapeutic efficacy, i.e., an interaction exists between the biomolecule and therapy that impacts patient outcome.

The term “priming” as used herein refers to the first encounter with a given antigen, which generates a primary adaptive immune response. The term “unprimed cells” (also referred to as virgin, naïve, or inexperienced cells) as used herein refers to T cells and B cells that have generated an antigen receptor (TCR for T cells, BCR for B cells) of a particular specificity, but have never encountered the antigen. For example, before helper T cells and B cells can interact to produce specific antibody, the antigen-specific T cell precursors must be primed.

Priming involves several steps: antigen uptake, processing, and cell surface expression bound to class II MHC molecules by an antigen presenting cell, recirculation and antigen-specific trapping of helper T cell precursors in lymphoid tissue, and T cell proliferation and differentiation. [Janeway, C A, Jr., “The priming of helper T cells, Semin. Immunol. (1989) 1 (1): 13-20]. Helper T cells express CD4, but not all CD4 T cells are helper cells. [Id.] The signals required for the clonal expansion of helper T cells differ from those required by other CD4 T cells. The critical antigen-presenting cell for helper T cell priming appears to be a macrophage; and the critical second signal for helper T cell growth is the macrophage product interleukin 1 (IL-1). [Id.] If the primed T cells and/or B cells receive a second, co-stimulatory signal, they become activated T cells or B cells.

The term “prognostic biomarker” refers to an indicator of innate tumor aggressiveness, is a biomolecule that indicates patient survival independent of the treatment received.

The term “PD-1” or “programmed cell death protein 1” as used herein refers to an inhibitory receptor expressed on the surface of activated T cells. Its ligands, PD-L1 and PD-L2, are expressed on the surface of DCs or macrophages. PD-1 and its ligands PD-L1/PL-L2 act as co-inhibitory factors that can limit the development of the T cell response. PD-L1 is overexpressed on tumor cells or on non-transformed cells in the tumor microenvironment [Pardoll, DM. Nat. Rev. Cancer (2012) 12: 252-64]. PD-L1 expressed on the tumor cells binds to PD-1 receptors on the activated T cells, which leads to the inhibition of the cytotoxic T cells. These deactivated T cells remain inhibited in the tumor microenvironment.

The term “protective immune response” or “protective response” as used herein, is meant to refer to an immune response mediated by antibodies against an infectious agent, which is exhibited by a vertebrate (e.g., a human), that prevents or ameliorates an infection or reduces at least one symptom thereof. The term can also refer to an immune response that is mediated by T-lymphocytes and/or other white blood cells against an infectious agent, exhibited by a vertebrate (e.g., a human), that prevents or ameliorates a viral infection or reduces at least one symptom thereof.

As used herein, the term “purify” is meant to refer to freeing from extraneous or undesirable elements.

The term “reads” as used in next-generation sequencing, refers to the DNA sequence from one fragment (meaning a small section of DNA). Next-generation sequencing read length refers to the number of base pairs (bp) sequenced from a DNA fragment. After sequencing, the regions of overlap between reads are used to assemble and align the reads to a reference genome, reconstructing the full DNA sequence.

The term “reduce” and its various grammatical forms as used herein refers to a diminution, a decrease, an attenuation or abatement of a degree, intensity, extent, size, amount, density or number.

The term “refractory” as used herein refers to a disease or condition that does not respond to treatment. A “refractory cancer” or “resistant cancer” is a cancer that does not respond to treatment. The cancer may be resistant at the beginning of treatment or it may become resistant during treatment.

The term “relapse” as used herein refers to the return of a disease or the signs and symptoms of a disease after a period of improvement. In cancer, the terms “relapse-free survival” or “RFS” and “disease free survival” or “DFS” refer to the length of time after primary treatment for a cancer ends that the patient survives without any signs or symptoms of that cancer.

The term “remission” as used herein refers to a decrease in or disappearance of signs and symptoms of cancer. In partial remission, some, but not all, signs and symptoms of cancer have disappeared. In complete remission, all signs and symptoms of cancer have disappeared, although cancer still may be in the body.

The term “restore” and its various grammatical forms as used herein refers to bringing back to a former or normal condition, to recover or renew.

The term “secondary lymphoid tissues” as used herein refers to sites where lymphocytes interact with each other and nonlymphoid cells to generate immune responses to antigens. These include the spleen, lymph nodes, and mucosa-associated lymphoid tissues (MALT).

As used herein, the term “secretion” and its various grammatical forms is meant to refer to production by a cell of a physiologically active substance and its movement out of the cell in which it is formed.

The term “senescence” as used herein refers to a biological process by which cells undergo growth arrest after extensive replication.

The term “stimulate an immune cell” or “stimulating an immune cell” as used herein is meant to refer to a process (e.g., involving a signaling event or stimulus) causing or resulting in a cellular response, such as activation and/or expansion, of an immune cell, e.g. a CD8+ T cell.

The term “subject” as used herein is meant to refer to any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The present disclosure above is intended for use in any of the above vertebrate species, since the immune systems of all of these vertebrates operate similarly.

The terms “T cell” or “T lymphocyte” are used interchangeably to refer to cells that mediate a wide range of immunologic functions, including the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. T cells recognize antigens on the surface of antigen presenting cells (APCs) and mediate their functions by interacting with, and altering, the behavior of these APCs. T cells can also be classified based on their function as helper T cells; T cells involved in inducing cellular immunity; suppressor T cells; and cytotoxic T cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC).

Although the lineage relationship between T cell subsets remains controversial, T cells cluster in populations that can be arranged as a progressive continuum on the basis of phenotypic, functional and transcriptional attributes. T lymphocytes transition through progressive stages of differentiation that are characterized by a stepwise loss of functional and therapeutic potential in the order from naive T (T_{N}) cells to T_{stem cell memory} (T_{SCM}) (the most immature antigen experienced T cells), to T_{central memory} (T_{CM}) cells, which patrol central lymphoid organs, to T_{effector memory} (T_{EM}) cells, which patrol peripheral tissues. In contrast to T_{N} cells, memory T cells are capable of rapidly releasing cytokines on restimulation. T_{CM} cells more efficiently secrete IL-2 and T_{EM} have an increased capacity for IFNγ release and cytotoxicity. All antigen-experienced T cells upregulate the common IL-2 and IL-15β receptor (IL-2RB) conferring the ability to undergo homeostatic proliferation in response to IL-15, and also display high amounts of CD95 (also known as FAS), a receptor that provides either costimulatory or pro-apoptotic signals depending on the efficiency of CD95 signaling complex formation and on which particular intracellular signaling proteins are part of the complex. [Gattinoni, L. et al. Nature Revs. Cancer 12: 671-684].

The term “T cell antigen” as used herein is meant to refer to a protein or fragment thereof which can be processed into a peptide that can bind to either Class I MHC, Class II MHC, non-classical MHC, or CD1 family molecules (collectively antigen presenting molecules), and in this combination can engage a T cell receptor on a T cell.

The term “T cell epitope” as used herein is meant to refer to a short peptide molecule that binds to a class I or II MHC molecule and that is subsequently recognized by a T cell. T cell epitopes that bind to class I MHC molecules are typically 8-14 amino acids in length, and most typically 9 amino acids in length. T cell epitopes that bind to class II MHC molecules are typically 12-20 amino acids in length. In the case of epitopes that bind to class II MHC molecules, the same T cell epitope may share a common core segment, but differ in the length of the carboxy- and amino-terminal flanking sequences due to the fact that ends of the peptide molecule are not buried in the structure of the class II MHC molecule peptide-binding cleft as they are in the class I MHC molecule peptide-binding cleft.

The term “T cell exhaustion” as used herein refers to a state of T cell dysfunction that arises during many chronic infections and cancer. It is defined by poor effector function, sustained expression of inhibitory receptors and a transcriptional state distinct from that of functional effector or memory T cells. Modulating pathways overexpressed in exhaustion—for example, by targeting programmed cell death protein 1 (PD1) and cytotoxic T lymphocyte antigen 4 (CTLA4)—can reverse this dysfunctional state and reinvigorate immune responses [Wherry E J and Kurachi, M. Nature (2015) 15: 486-499, citing Wherry E J. Nat. Immunol. (2011) 131: 492-499; Schietinger A. and Greenberg P D. Trends Immunol. (2014) 35: 51-60; Barber D L, et al. Nature. (2006) 439: 682-687; Nguyen L T. and Ohashi P S. Nat. Rev. Immunol. (2014) 15: 45-56]. The level and duration of chronic antigen stimulation and infection seem to be key factors that lead to T cell exhaustion and correlate with the severity of dysfunction during chronic infection. Examples of inhibitory receptors include the inhibitory pathways mediated by PD1 in response to binding of PD1 ligand 1 (PDL1) and/or PDL2. [Id., citing Okazaki T, et al., Nature Immunol. (2013) 14: 1212-1218, Odorizzi P M. and Wherry E J. J. Immunol. (2012) 188: 2957-2965, Araki K, et al. Cold Spring Harb. Symp. Quant. Biol. (2013) 78: 239-247]. Exhausted T cells can co-express PD1 together with lymphocyte activation gene 3 protein (LAG3), 2B4 (also known as CD244), CD160, T cell immunoglobulin domain and mucin domain-containing protein 3 (TIM3; also known as HAVCR2), CTLA4 and many other inhibitory receptors [Id., citing Blackburn S D, et al. Nat. Immunol. (2009) 10: 29-37]. Typically, the higher the number of inhibitory receptors co-expressed by exhausted T cells, the more severe the exhaustion. It has been suggested that inhibitory receptors such as PD1 might regulate T cell function in several ways [Id., citing Schietinger A. and Greenberg P D. Trends Immunol. (2014) 35: 51-60; Odorizzi P M. and Wherry E J. J. Immunol. (2012) 188: 2957-2965], e.g., by ectodomain competition, which refers to inhibitory receptors sequestering target receptors or ligands and/or preventing the optimal formation of microclusters and lipid rafts (for example, CTLA4); second, through modulation of intracellular mediators, which can cause local and transient intracellular attenuation of positive signals from activating receptors such as the TCR and co-stimulatory receptors [Id., citing Parry R V, et al. Molec. Cell. Biol. (2005) 25: 9543-9553; Yokosuka T, et al. J. Exp. Med. (2012) 209: 1201-1217; Clayton K L, et al. J. Immunol. (2014) 192: 782-791]; and third, through the induction of inhibitory genes [Id., citing Quigley M, et al. Nat. Med. (2010) 16: 1147-1151]. Co-stimulatory receptors also are involved in T cell exhaustion [Id., citing Odorizzi P M. and Wherry E J. J. Immunol. (2012) 188: 2957-2965]. It has also been possible to exploit the potential beneficial role of co-stimulation to reverse exhaustion by combining agonistic antibodies to positive co-stimulatory pathways with blockade of inhibitory pathways. 4-1BB (also known as CD137 and TNERSF9) is a TNFR family member and positive co-stimulatory molecule that is expressed on activated T cells. Combining PD1 blockade and treatment with an agonistic antibody to 4-1BB dramatically improved exhausted T cell function and viral control [Id, citing Vezys V, et al. J. Immunol. (2011) 187: 1634-1642]. Soluble molecules are a second class of signals that regulate T cell exhaustion; these include immunosuppressive cytokines such as IL-10 and transforming growth factor-β (TGFβ) and inflammatory cytokines, such as type I interferons (IFNs) and IL-6. [Id.]

The term “T cell mediated immune response” as used herein is meant to refer to a response that occurs as a result of recognition of a T cell antigen bound to an antigen presenting molecule on the cell surface of an APC, coupled with other interactions between costimulatory molecules on the T cell and APC. This response serves to induce T cell proliferation, migration, and production of effector molecules, including cytokines and other factors that can injure cells.

The term “T cell receptor” (TCR) as used herein, is meant to refer to a complex of integral membrane proteins that participate in the activation of T cells in response to an antigen. The TCR expressed by the majority of T cells consisting of a heterodimer of α and β chains. A small group of T cells express receptors made of γ and δ chains. Among the α/β T cells are two sublineages: those that express the coreceptor molecule CD4 (CD4+ cells), and those that express CD8 (CD8+ cells). These cells differ in how they recognize antigen and in their effector and regulatory functions. CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated. The cytokines can be directly toxic to target cells and can mobilize potent inflammatory mechanisms. CD8+ T cells, can develop into cytotoxic T-lymphocytes (CTLs) capable of efficiently lysing target cells that express antigens recognized by the CTLs.

Naive conventional CD4 T cells can differentiate into four distinct T cell populations, a process that is determined by the pattern of signals they receive during their initial interaction with antigen. These 4 T cell populations are TH1, TH2, TH17, and induced regulatory T (iTreg) cells. TH1 cells, which are effective inducers of cellular immune responses, mediate immune responses against intracellular pathogens, and are responsible for the induction of some autoimmune diseases. Their principal cytokine products are IFNγ (which enhances several mechanisms important in activating macrophages to increase their microbiocidal activity), lymphotoxin α (LTα), and IL-2, which is important for CD4 T cell memory. TH2 cells, which are effective in helping B cells develop into antibody producing cells, mediate host defense against extracellular parasites, are important in the induction and persistence of asthma and other allergic disease, and produce IL-4, IL-5, IL-9, IL-10 (which suppresses TH1 cell proliferation and can suppress dendritic cell function), IL-13, IL-25 (signaling through IL-17RB, enhances the production of IL-4, IL-5, and IL-13 by a c-kit-FcεRI-nonlymphocyte population, serves as an initiation factor as well as an amplification factor for TH2 responses) and amphiregulin. IL-4 and IL-10 produced by TH2 cells block IFNγ production by T_{H}1 cells. TH17 cells produce IL-17a, IL-17f, IL-21, and IL-22. IL-17a can induce many inflammatory cytokines, IL6 as well as chemokines such as IL-8 and plays an important role in inducing inflammatory responses. Treg cells play a critical role in maintaining self-tolerance and in regulating immune responses. They exert their suppressive function through several mechanisms, some of which require cell-cell contact. The molecular basis of suppression in some cases is through their production of cytokines, including TGFβ, IL-10, and IL-35. TGFβ produced by T reg cells may also result in the induction if iTreg cells from naïve CD4 T cells. CD4+ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. Zhu, J. and Paul, W E, Blood (2008) 112: 1557-1569). Resting naïve CD8+ T cells, when primed by antigen presenting cells that have acquired antigens from the infected macrophages through direct infection or cross-presentation in secondary lymphoid organs, such as lymph nodes and spleen, react to pathogens by massive expansion and differentiation into cytotoxic T lymphocyte effector cells that migrate to all corners of the body to clear the infection. In the majority of viral infections, however, CD8 T cell activation requires CD4 effector T cell help to activate dendritic cells for them to become able to stimulate a complete CD8 T cell response. CD4 T cells that recognize related antigens presented by the APC can amplify the activation of naïve CD8 T cells by further activating the APC. B7 expressed by the dendritic cell first activates the CD4 T cells to express IL-2 and CD40 ligand. CD40 ligand binds CD40 on the dendritic cell, delivering an additional signal that increases the expression of B7 and 4-1BBL by the dendritic cell, which in turn provides additional co-stimulation to the naïve CD8 T cell. The IL-2 produced by activated CD4 T cells also acts to promote effector CD T cell differentiation.

The term “TCR/CD3 complex” as used herein refers to a protein complex composed of four distinct chains. In mammals, the complex contains a CD3γ chain, a CD38 chain, and two CD3ε chains, which associate with the T cell receptor (TCR) and the ζ-chain to generate an activation signal in T lymphocytes. Together, the TCR, the ζ-chain and CD3 molecules comprise the TCR complex. The intracellular tails of CD3 molecules contain a conserved motif known as the immunoreceptor tyrosine-based activation motif (ITAM), which is essential for the signaling capacity of the TCR. Upon phosphorylation of the ITAM, the CD3 chain can bind ZAP70 (zeta associated protein), a kinase involved in the signaling cascade of the T cell.

The term “T follicular helper (TFH) cells” as used herein refers to a distinct subset of CD4+ T lymphocytes, specialized in B cell help and in regulation of antibody responses. They develop within secondary lymphoid organs (SLO) and can be identified based on their unique surface phenotype, cytokine secretion profile, and signature transcription factor. They support B cells to produce high-affinity antibodies toward antigens, in order to develop a robust humoral immune response and are crucial for the generation of B cell memory. They are essential for infectious disease control and optimal antibody responses after vaccination. Stringent control of their production and function is critically important, both for the induction of an optimal humoral response against thymus-dependent antigens but also for the prevention of self-reactivity. [Gensous, N. et al. Front. Immunol. (2018) doi.org/10.3389/fimmu.2018.01637).

The term “T_{H}1 cells” as used herein refers to a lineage of CD4+ effector T cells that promotes cell-mediated immune responses and is required for host defense against intracellular viral and bacterial pathogens. They are mainly involved in activating macrophages but can also help stimulate B cells to produce antibody. T_{H}1 cells secrete IFN-gamma, IL-2, IL-10, and TNF-alpha/beta. IL-12 and IFN-γ make naive CD4+ T cells highly express T-bet and STAT4 and differentiate to T_{H}1 cells. (Zhang, Y. et al. Adv. Exp. Med. Bio. (2014) 841: 15-44).

The term “T_{H}2 cells” as used herein refers to a lineage of CD4+ effector T cells that secrete IL-4, IL-5, IL-9, IL-13, and IL-17E/IL-25. These cells are required for humoral or antibody-mediated immunity and play an important role in coordinating the immune response to large extracellular pathogens. IL-4 makes naive CD4+ T cells highly express STAT6 and GATA3 and differentiate to T_{H}2 cells. (Zhang, Y. et al. Adv. Exp. Med. Bio. (2014) 841: 15-44).

The term “T_{H}17 cells” as used herein refers to a CD4+ T-cell subset characterized by production of interleukin-17 (IL-17). IL-17 is a highly inflammatory cytokine with robust effects on stromal cells in many tissues, resulting in production of inflammatory cytokines and recruitment of leukocytes, especially neutrophils, thus creating a link between innate and adaptive immunity. [Tesmer, L A, et al., Immunol. Rev. (2008) 223: 87-113]. The key transcription factor in T_{H}17 cell development is RORγt.

The term “Treg” or “regulatory T cells” as used herein refers to effector CD4 T cells that inhibit T cell responses and are involved in controlling immune reactions and preventing autoimmunity. The natural regulatory T cell lineage that is produced in the thymus is one subset. The induced regulatory T cells that differentiate from naïve CD4 T cells in the periphery in certain cytokine environments is another subset. Tregs are most commonly identified as CD3+CD4+CD25+FoxP3+ cells in both mice and humans. Additional cell surface markers include CD39, 5′ Nucleotidase/CD73, CTLA-4, GITR, LAG-3, LRRC32, and Neuropilin-1. Tregs can also be identified based on the secretion of immunosuppressive cytokines including TGF-beta, IL-10, and IL-35. Cell surface molecules CTLA-4, LAG-3, and neuropilin-1 (Nrp1) impair dendritic cell (DC)-mediated conventional T cell activation: CTLA-4 and LAG-3 outcompete CD28 and T cell receptor expressed on conventional T cells for binding to CD80/86 and MHC class II on DCs, and Nrp1 stabilizes DC-Treg contact, thereby preventing antigen presentation to conventional T cells [Ikebuchi, R. et al. Front. Immunol. (2019) doi.org/10.3389/fimmu.2019.01098].

The term “TIGIT” refers to a member of the Ig super family and an immune inhibitory receptor.

The term “TIM-3” as used herein refers to a transmembrane protein and immune checkpoint receptor. It is associated with tumor-mediated immune suppression.

The term “tissue-resident memory T cell” or “TRM” as used herein refers to memory lymphocytes that do not migrate after taking up residence in barrier tissues, where they are retained long term. They appear to be specialized for rapid effector function after restimulation with antigen or cytokines at sites of pathogen entry.

The term “tolerance” as used herein refers to the failure to respond to a particular antigen. Tolerance mechanisms that operate in the thymus before the maturation and circulation of T cells are referred to as “central tolerance.” Not all antigens of which T cells need to be tolerant are expressed in the thymus, and therefore central tolerance mechanisms alone are insufficient. Additional tolerance mechanisms exist to restrain the numbers and or function of T cells that are reactive to developmental or food antigens, which are not thymically expressed. Tolerance acquired by mature circulating T cells in the peripheral tissues is called “peripheral tolerance.”.

The term “toll-like receptor (TLR)” as used herein refers to innate receptors on macrophages, dendritic cells, and some other cells, that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

The term “transarterial embolization” or “TAE” as used herein refers to procedure in which the blood supply to a tumor or an abnormal area of tissue is blocked. The mechanism by which arterial embolization preferentially kills HCC but spares adjacent liver tissues arises from the dual blood supply from the portal vein (PV) and the remaining 25% from the hepatic artery (HA). In contrast, HCC almost exclusively receives its blood supply from the HA. Based on this pattern, embolization has been used to selectively block the arterial blood supply to HCC, causing transient but profound ischemia and depriving HCC of essential oxygen and nutrients, thus killing the tumors. (Lin, 2016). However, because of the heterogeneity of the tumor vessels within HCC, the embolization of the tumor-feeding arteries usually results in different degrees of ischemia and hypoxia, ranging from 0.1 to 10 μM oxygen in HCC after embolization. [Lin, W H, et al. Proc. Nat'l. Acad. Sci. USA (2016) 113 (42): 11937-11942].

The term “transarterial chemoembolization” or “TACE” as used herein refers to a procedure that places chemotherapy and embolic agents into a blood vessel feeding a cancerous tumor to cut off the tumor's blood supply and trap the chemotherapy within the tumor.

The term “transarterial tirapazamine embolization” or “TATE” as used herein refers to a treatment procedure in which transarterial embolization is combined with treatment with tirapazamine.

The term THIA as used herein refers to a “tumor hypoxia-inducing agent”, meaning an agent that selectively targets tumor vessels without damaging normal vasculature. Exemplary tumor hypoxia-inducing agents include, without limitation, Combretastatin A4 (CA4) (U.S. Pat. No. 4,996,237), prodrug Combretastatin A4P (U.S. Pat. No. 5,561,122), halogen derivatives (U.S. Pat. No. 7,223,747), and various derivatives of CA4 as described in U.S. Pat. No. 8,853,270, etc.

The term “toxicity” as used herein refers to the degree to which a substance can harm humans or animals. Acute toxicity involves harmful effects in an organism through a single or short-term exposure.

The term “tumor associated antigen” or “TAA” refers to a protein or other molecule that has elevated levels on tumor cells but that is also expressed at lower levels on healthy cells. Tumor specific antigens (“TSA”) are found on cancer cells only.

The term “tumor associated macrophages” or “TAMs” as used herein refers to an immunosuppressive macrophage subtype found in the tumor microenvironment that is involved in the progression and metastasis of cancer. TAMs are broadly considered M2-like, which can be further classified into the M2a phenotype (induced by IL-4 or IL-13), M2b phenotype (IL-10 high, IL-12 low) and M2c phenotype (TNF-α low) according to distinct signal stimuli. They produce abundant growth factors, extracellular matrix (ECM) remodeling molecules and cytokines for the regulation of cancer proliferation via noncoding RNAs, exosomes and epigenetics [Yan, S. and Wan, G. The FEBS Journal (2021) 288 (21): 6174-6186, citing Qian, B Z and Pollard, J W. Cell (2010) 141: 39-51]. Activated M2 macrophages distinctively express arginase 1 (ARG1). TAMs can demonstrate direct inhibition on the cytotoxicity of T-lymphocyte through multiple mechanisms and characteristics of tumor evolution, including immune checkpoint engagement via expression, production of inhibitory cytokines [such as IL-10 and transforming growth factor (TGF)-β] and metabolic activities consisting of depletion of 1-arginine (or other metabolites) and the production of reactive oxygen species (ROS). The suppressive immune response renders cancer cells capable of escaping from immune surveillance.

The term “tumor infiltrating lymphocytes” as used herein refers to a heterogeneous lymphocyte population mainly composed of T lymphocytes that may consist of numerous antitumor effector and/or regulatory T cells (Tregs) and are key players in the host's immune response to a tumor. [Wang, J. et al. BMC Cancer (2020) 20: 731].

The term “tumor microenvironment” or “TME” refers to the cellular environment in which tumors or cancer stem cells exist.

The term “TME macrophages” as used herein refers to macrophages that arise primarily from bone marrow-derived monocytes that are recruited by tumor or stroma-derived chemokines such as colony-stimulating factor 1 (CSF1; also known as M-CSF) and CCL2. M1 and M2 phenotypes are differentiated in response to different signal stimuli and are polarized according to the TME, exhibiting strong plasticity, such that macrophages adopt context-dependent phenotypes when stimulated [Yan, S. and Wan, G. The FEBS Journal (2021) 288 (21): 6174-6186, citing Murry, P J and Wynn, TA. Nat. Rev. Immunol. (2011) 11: 723-737]. Antitumorigenic M1 macrophages express high levels of tumor necrosis factor (TNF), inducible nitric oxide synthase (iNOS; also known as NOS2) and major histocompatibility complex (MHC) class II molecules, whereas pro-tumorigenic M2 macrophages are marked with high levels of arginase 1 (ARG1), interleukin (IL)-10, CD163, CD204 or CD206 expression. The activation of primary macrophages into M1 or M2 phenotype is mainly induced by interferon-regulatory factor/signal transducer and activator of transcription signaling pathways [Id., citing Waqas, S F H et al. in Nuclear Receptors: Methods and Experimental Protocols, M Z Badr. Ed., Springer, New York, NY, pp. 211-224].

The term “tumor necrosis-inducing agent” (“TUNIA”) as used herein refers to an agent that sensitizes tumor cells to necrosis cell death.

The terms “tumorigenesis” “oncogenesis” and “carcinogenesis” are used interchangeably to refer to the transformation of normal cells into cells-of-origin (COOs) and the development of cells-of-origin into tumors.

The terms “variants”, “mutants”, and “derivatives” are used herein to refer to nucleotide or polypeptide sequences with substantial identity to a reference nucleotide or polypeptide sequence. The differences in the sequences may be the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence. A skilled artisan likewise can produce polypeptide variants having single or multiple amino acid substitutions, deletions, additions or replacements, but biologically equivalent to the wild type sequence. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, for example, an epitope for an antibody. The techniques for obtaining such variants, including, but not limited to, genetic (suppressions, deletions, mutations, etc.), chemical, and enzymatic techniques, are known to the skilled artisan.

The term “wild type” as used herein refers to the typical form of an organism, strain, gene, protein, nucleic acid, or characteristic as it occurs in nature. Wild type refers to the most common phenotype in the natural population.

The term “whole blood” as used herein refers to generally unprocessed or unmodified blood collected from a subject containing all of its components, including, but are not limited to, plasma, cellular components (e.g., red blood cells, white blood cells (including lymphocytes, monocytes, eosinophils, basophils, and neutrophils), and platelets), proteins (e.g., fibrinogen, albumin, immunoglobulins), hormones, coagulation factors, and fibrinolytic factors. The term “whole blood” is inclusive of any anticoagulant that may be combined with the blood upon collection.

### EMBODIMENTS

The present disclosure describes a way to solve the problem in identification of tumor-specific antigens by identification of a TCR that is known to be from effective anti-tumor T cells. The availability of an effective TCR can be used directly in building TCR or CAR-T as a type of cell therapy. The platform immunotherapy can be offered to cancer patients with a variety of immunotherapy approaches.

According to a first aspect, patients' circulatory T cells are examined to identify any clonal expansion of T cells resulting from treatment with a tumor necrosis inducing agent.

The following is an exemplary process.

Blood sample collection: A sample method used to collect blood spots is as follows. A finger of a subject is pricked in order to deposit blood onto a sample collecting filter, filling four circles approximately 1-2 cm in diameter completely. The sample card is then stored in a plastic bag with a desiccant and shipped back to the laboratory for processing once the protocol is complete. Alternatively, a blood sample is collected in a PAXgene vacutainer tube. RNA is prepared and shipped to the laboratory on dry ice.

In step 1, samples are extracted to obtain nucleic acids (DNA or RNA). Following extraction, the amount and quality of DNA or RNA is determined as an A260/280 value. “Pure” DNA generally reports an A260/A280 reading of 1.8; whereas RNA is closer to 2.0. A sequencing library is prepared from the RNA or DNA sample by (1) amplification to yield a pool of appropriately sized target sequences; and (2) the addition of sequencing adapters that later will interact with the next generation sequencing (NGS) platform. If RNA is the starting template, the RNA is first converted to cDNA by reverse transcription.

In step 2, PCR amplification yields a collection of specifically sized DNA fragments (a library) that are compatible with the sequencing system to be used. The adapter ligation step bookends the amplified DNA or cDNA fragments, called amplicons, with specific oligonucleotide sequences that will interact with the surface of a sequencing flow cells. If multiple samples are to be sequenced in a single sequencing run, a unique identifier (or barcode) is additionally ligated to the amplicon. The resulting completed libraries can be pooled into a single sequencing run that is then “demultiplexed” during data analysis.

By conducted NGS of the circulated T cells, the TCR of the expanded clones can be identified. In step 3, parallel sequencing is performed using an NGS platform. The library is loaded onto the sequencer which then “reads” the nucleotides one by one. In step 4, after sequencing is complete, first, the reads are filtered for quality, amplicon size, and agreement between paired ends. The reads then are assembled and aligned to a reference genome. Finally, reads (assembled or raw) are compared to a reference sequence or to reads from another sample to identify variants. If reads are aligned with a reference genome, variant annotation can be used to associate variants with known genes or regulatory sequences.

According to a second aspect, CD4+, CD8+ or both CD4+ and CD8+ T cells of the patient can be transduced to express a T cell receptor comprising the sequence of the complementarity-determining region (CDR) of the TCR specific for the tumor-specific antigen. Each TCR V domain (α and β) contains three complementarity determining region (CDR) loops that combine to form the TCR binding interface.

The transduced T cells expressing the TCR specific for the tumor-specific antigen then can be expanded to a sufficient quantity for administration to the patient.

According to some embodiments, the TCR specific for the tumor-specific antigen is a chimeric antigen receptor. The term “chimeric antigen receptor” or “CAR” as used herein refers to a synthetic MHC-independent receptor that targets T cells to a chosen antigen and reprograms T cell function, metabolism, and persistence [Riviere, I. and Sadelain, M. Mol. Ther. (2017) 25 (5): 1117-1124, citing Eshhar, Z. et al. Springer Semin. Immunopathol. (1996) 18: 199-209; Sadalain, M. et al. Nat. Rev. Cancer (2003) 3: 35-45]. A CAR is mainly composed of three parts: an extracellular antigen recognition domain, usually a single-chain variable fragment (scFv) derived from a monoclonal antibody; a spacer/hinge region and transmembrane domain; and an intracellular signal transduction domain.

A CAR constructed in this manner will specifically bind an antigen expressed exclusively in tumor cells to form an effective immunological synapse leading to downstream T-cell signaling and a potent and specific anti-tumor effect.

Through their extracellular domain, CARs bind cell surface molecules independently of the major histocompatibility complex (MHC), in contrast to the physiological T cell receptor, which engages MHC/peptide complexes. CARs may thus target proteins, carbohydrates, or glycolipids and function independently of patient HLA haplotype. Binding to antigen triggers T cell activation, which is commonly mediated by the cytoplasmic domain of the CD3-ζ chain. [Riviere, I. and Sadelain, M. Mol. Ther. (2017) 25 (5): 1117-1124, citing Irving, B A and Weiss, A. Cell (1991) 64: 891-901; Romeo, C., Seed, B. Cell (1991) 64: 1037-1046; Letourneur, F. and Klausner, RD. Proc. Natl. Acad. Sci. USA (1991) 88: 8905-8909; Eshhar, Z. et al. Proc. Natl Acad. Sci. USA (1993) 90: 720-724; Brocker, T. et al. Eur. J. Immunol. (1993) 23: 1435-1439].

Merely providing T cell activation is, however, not sufficient to direct a productive immune response. [Id., citing Brocher, T. and Karjalainen, K. J. Exp. Med. (1995) 181: 1653-1659; Gong, M M C et al. Neoplasia (1999) 1: 123-127; Brocker, T. Blood (2000) 96: 1999-2001]. The CARs that have provided tangible clinical benefits incorporate a costimulatory domain [Id., citing Krause, A. et al. J. Exp. Med. (1998) 188: 619-626], which enables T cells to expand and retain their functionality upon repeated exposure to antigen. [Id., citing Maher, J. et al. Nat. Biotechnol. (2002) 20: 70-75] These receptors have been dubbed second generation CARs [Id., citing Sadelain, M. et al. Curr. Opinion. Immunol. (2009) 21: 215-233] and are key to the design of persisting engineered T cells that can attack tumors as long as they retain their functionality. Several recent reviews have addressed CAR design [Id., citing Jensen, M C and Riddell, SR. Curr. Opinion. Immunol. (2015) 33: 9-15; van der Stegan, S J et al. Nat. Rev. Drug Discov. (2015) 14: 499-509; Sadelain, M. J. Clin. Invest. (2015) 125: 3392-3400; Maus, M V and June, CH. Clin. Cancer Res. (2016) 22: 1875-1884], CAR prospects for solid tumors, [Id., citing Hinrichs, C S and Restifo, NP Nat. Biotechnol. (2013) 999-1008; Morello, A. et al. Cancer Discov. (2016) 6: 133-46] and T cell manufacturing. [Id., citing Wang, X. and Riviere I., Mol. Ther. Oncolytics. (2016) 3: 16015; Levine, B L et al. Mol. Ther. Methods Clin. Dev. (2016) 4: 92-101; Wang, X., Riviere, I. Cancer Gene Ther. (2015) 22: 85-94].

According to a third aspect, the identity of the tumor-specific antigen will be determined from the identified CDR in the anti-tumor T cells, the patient's tumor mutation derived from NGS, and the MHC information by a virtual screening of a peptide library in silica by the generative AI approach.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Isolation of Peripheral Blood Mononuclear Cells (PBMCs) for NGS Analysis

Cancer patients are treated with a tumor necrosis-inducing agent (TUNIA) for tumor debulking and induction of tumor necrosis. According to some embodiments, PBMCs containing CD4+ T cells, CD8+ T cells, or both CD4+ and CD8+ T cells are then isolated; the expanded CD8+ T cells in the PBMCs must be fully active and not an exhausted T-cell population as evidenced by, for example, co-expression of multiple inhibitory receptors (e.g., PD-1. CTLA-4, LAG-3, TIM-3, 2B4/C244/SLAMF4; CD160, TIGIT, loss of IL-2 production, proliferative capacity, ex vivo cytolytic activity; impairment in the production of TNF-alpha, IFN-gamma, and cc (beta) cytokines.

PBMCs will be subject to red blood cell (RBC) lysis, and T cells then will be isolated using affinity magnetic beads to pull down the T cells. The PBMC layer or T cell population will be collected and the cell RNA then extracted. The isolated RNA will be subjected to NGS analysis using TCR-specific primers to amplify the CDR region that is critical in coding the antigen-binding domain.

For every patient from whom the sequence of TCR from active anti-tumor T cells will be isolated, the patient will receive an anti-PD-1 immune checkpoint inhibitor, such as pembrolizumab or nivolumab, at day 1. Then the TUNIA treatment, such as the TATE procedure if there is liver involvement, will be conducted in Day 8. The same antibody against an immune checkpoint inhibitor, such as pembrolizumab or nivolumab, will be continued per standard dosing regimens for at least 4 doses every 3 weeks. Blood samples at four time points will be collected for RNA isolation. Pre-TATE treatment samples (14 mL of peripheral venous blood or 2 tubes of green top tubes) will be collected on day 8 before the TUNIA treatment, such as the TATE procedure. Three post-TATE blood samples will be collected on Days 22, 43 and 64, before nivolumab is administered. At each time point, two green-top tubes of venous blood will be collected (7 mL each tube). Each patient will contribute 56 mL in total for this step. The collected blood samples will be mixed with EDTA for anti-coagulation and sent to the central laboratory in ice for processing and isolation of plasma, and PBMCs. The isolated plasma (in two 1 mL aliquots per tube) will be centrifuged in a tabletop centrifuge at maximal speed (14,000 rpm) for another 10 min at 4° C. to remove all cellular components. The supernatant is frozen in −80° C. for future cytokine and circulating DNA analysis. PBMCs will be mixed with a suitable storage reagent, such as Trizol or RNA-later solution, for future DNA and RNA extraction and TCR sequencing.

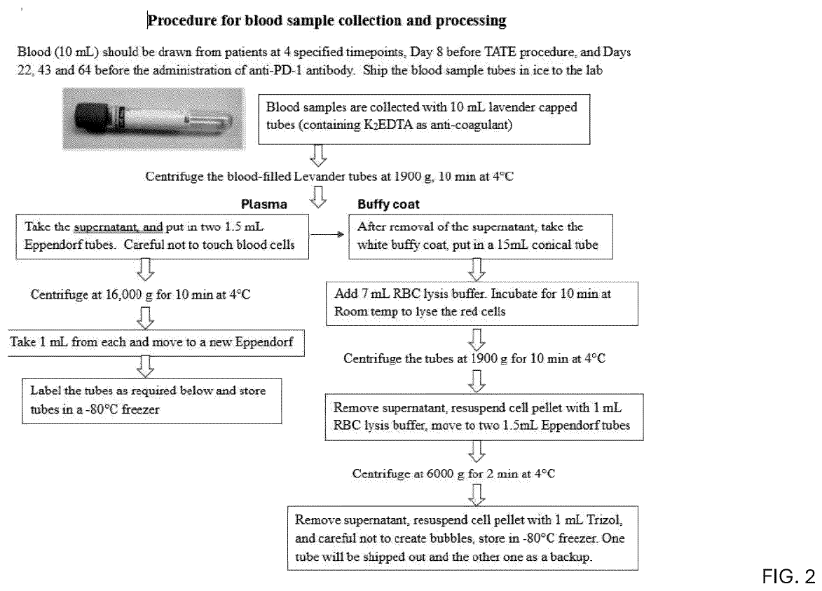

FIG. 2 is a flow chart showing the sample collection procedures.

### Example 2: Identification of the Sequence of TCR by NGS and Cloning into a TCR Full Length CAR-T Vector

TCR sequencing will be performed on the RNA samples as described by Danilova et al. [Danilova L., et al. Cancer Immunol. Res. (2018) 6 (8): 888-899]. Bioinformatic analysis will be performed and clustered to search for clonal expansion. A search for a clone of TCR sequence that fulfills the following criteria to represent an expanded anti-tumor immunity will be initiated:
(1). significant expansion (Fisher's exact test with Benjamini-Hochberg correction for FDR; p<0.05) in the post-treated sample compared to the pre-treated sample,
(2). Reach a minimum baseline threshold or a minimum of 10 templates detected by TCRseq in the post-treatment samples,
(3). Increase in at least post-treatment samples collected at 1 and 2 months (preferred) after tumor necrosis-inducing agent (TUNIA) treatment.

### Example 3: Potential Applications of TCR and Tumor-Specific Antigen as a Platform Immunotherapy for Cancer Patients

The following series of individualized cancer immunotherapy steps can be conducted to treat the cancer patient.
1. Tumor debulking with TUNIA. This step can be further synergized with addition of an immune checkpoint inhibitor, such as an anti-PD-(L) 1 monoclonal antibody.
2. During the treatment course with TUNIA and immune checkpoint inhibitor, peripheral blood of the patients will be collected to isolate PBMCs for isolation of RNA for NGS after amplification of the TCR using TCR-specific primers.
3. The identified TCR in the clonally expanded T cell population will be inserted into a full TCR or CAR-T vector for preparation of autologous cell therapy.
4. If the patient develops disease progression, the patient will have a leukapheresis to isolate his T cells and the T cells will be transfected with a TCR or CAR-T vector with >90% efficacy by mRNA. The T cells will be expanded in standard cell bio-processor to a sufficient number for administration back to the patient as a way of cell therapy.

Since each tumor-specific antigen is unique and unlikely to be shared among different patient populations, this therapy will be individualized. The initial cost would be high, but with technology advancement and improved cell therapy processes standardized, the cost of cell therapy is expected to drop quickly in the future so that more cancer patients could benefit from this multi-modality platform immunotherapy.

### Example 4. TACI (Tumor Activated Cellular Immunity)-Expansion of the Peripheral Blood Mononucleated Cells (PBMCs) after TATE for Ex Vivo Expansion to Treat Cancer Patients Who Received TATE+Anti-PD-1

### Definitions

The term “amplicon” as used herein refers to the end product of a replicated or amplified piece of DNA.

The term “CD4+ T cell” as used herein refers to the major regulatory cells of the immune system. Their regulatory function depends both on the expression of their cell-surface molecules, such as CD40 ligand whose expression is induced when the T cells are activated, and the wide array of cytokines they secrete when activated.

The term “CD8+ T cell” refers to cytotoxic T-lymphocytes (CTLs) capable of efficiently lysing target cells that express antigens recognized by the CTLs. [Paul, W. E., “Chapter 1: The immune system: an introduction,” Fundamental Immunology, 4th Edition, Ed. Paul, W. E., Lippincott-Raven Publishers, Philadelphia (1999)]. Activation of CD8+ T cells to CD 8+ effector cells entails a transition from naïve cells to a state where thousands of regions of DNA and chromatin are epigenetically modified to facilitate the drastic functional change. Upon clearance of the pathogen, most CD8+ T cells die. Long-lived memory CD8 T cells are derived from a subset of effector T cells through a process of dedifferentiation, whereby the subset of cells that gives rise to memory cells acquires de novo DNA methylation programs at naïve-associated genes and became demethylated at loci of classically defined effector molecules, thus retaining many epigenetic marks of their effector state. This epigenetic marking likely accounts for the ability of memory CD8+ Tcells to rapidly recall proliferation and effector function upon a second encounter with the pathogen. [Youngblood, B. et al. Nature (2017) 552 (7685): 404-409].

The term “clonal expansion” as used herein refers to rapid proliferation of a specific cell type.

The term “clonal selection” as used herein refers to the activation of only those clones of lymphocytes bearing receptors specific for a given antigen.

The term “clonotyping” as used herein refers to a process to identify the unique nucleotide CDR3 sequences of a TCR chain. This generally involves PCR amplification of the cDNA using V-region specific primers and either constant region (C) specific or J-region-specific primer pairs, followed by nucleotide sequencing of the amplicon. [Yassai, M B et al. Immnogenetics (2009) 61 (7): 493-502]

The term “cluster analysis” as used herein in the context of clonal expansion, is a computational method used to group together cells or DNA sequences that share a high degree of similarity, allowing researchers to identify populations of cells originating from a single progenitor cell (a clone), indicating clonal expansion.

The term “cognate antigen” as used herein refers to an antigen known to be recognized by a given lymphocyte antigen receptor because it was used for the original activation of that lymphocyte. On recognition of a cognate antigen, lymphocytes massively proliferate and generate expanded clones of required antigen reactivity, which establish an adequate specific immune response. Clonal expansion is coupled with differentiation of naïve lymphocytes into mature effector and memory cells. [Polonsky, M. et al. Immunol. And Cell Biol. (20016) 94: 142-249].

The term “combinatorial diversity” as used herein refers to a component of antibody and TCR diversity that is generated by the multiple different copies of each type of gene segment and junctional diversity introduced at the joints between the different gene segments as a result of addition and subtraction of nucleotides by the recombination process. A third source of diversity arises from the many possible different combinations of heavy- and light-chain V regions that pair to form the antigen-binding site in the immunoglobulin (Ig) molecule. The structural diversity of TCRs is attributable mainly to combinatorial and junctional diversity generated during the process of gene rearrangement. Most of the variability in T cell receptor chains is in the junctional regions, which are encoded by V, D and J gene segments. The TCRα locus contains many more J gene segments than either of the Ig light-chain loci. Because the TCRα locus has so many J gene segments, the variability generated in this region is even greater for TCRs than for Igs. Thus, most of the diversity resides in the CDR3 loops that contain the junctional region and form the center of the antigen-binding site. The CDR3 of a γ: δ cell is frequently longer than the CDR3 in an α: β T-cell receptor; this permits the CDR of γ: δ TCRs to interact directly with ligand and also contributes to the great diversity of these receptors. [Janeway's Immunobiology K. Murphy and C. Weaver Eds., 9th Edn. Garland Science: New York (2017), Chapter 5, pp. 180-191].

The term “complementarity-determining regions or “CDRs” as used herein refers to parts of the V domains of immunoglobulins and T cell receptors that determine their antigen specificity and make contact with the specific ligand. The CDRs are the most variable part of the antigen receptor and contribute to the diversity of these proteins. There are three such regions (CDR1, CDR2, and CDR3) in each V domain.

The term “diversity” as used herein refers to the number of classes, degree of dispersion among classes, species richness, variety, or a multiformity. For example, antibodies with an enormous diversity of antigen-binding sites are produced by B cells. Such antibody diversity is generated from the large number of V, J, D, and C genes that enable the immune system to generate an almost unlimited number of different light and heavy chains by joining these separate gene segments together before they are transcribed. The range of different TCRs expressed is likewise the result of recombination. TCRs are heterodimers that fall into two classes: TCR-αβ and TCR-γδ. The TCR α- and γ-chains constitute a variable (V), joining (J) and constant region (C). The TCR β- and δ-chains are also made up of a V, J and C region, with an additional diversity (D) region. One segment from each region is recombined, with additional nucleotide additions and/or deletions, to generate each rearranged TCR. This recombination generates high T-cell diversity and enables T cell recognition of millions of antigens.

The term “diversity measurement” refers to the number of species (clonotypes) present in a biological entity. [Aversa, I. et al. Intl J. Molec. Sci. (2020) 21: 238].

The term “immunological repertoire” refers to the collection of transmembrane antigen-receptor proteins located on the surface of T and B cells. (Benichou, J. et al. Immunology (2011) 135: 183-191). The combinatorial mechanism that is responsible for encoding the receptors does so by reshuffling the genetic code, with a potential to generate more than 10E18 different T cell receptors (TCRs) in humans (Id., citing Venturi, Y. et al. Nat. Rev. Immunol. (2008) 8: 231-238), and a much more diverse B-cell repertoire. These sequences, in turn, will be transcribed and then translated into protein to be presented on the cell surface. The recombination process that rearranges the gene segments for the construction of the receptors is key to the development of the immune response, and the correct formation of the rearranged receptors is critical to their future binding affinity to antigen. (Id.) The highly diverse junctional region of the TCR chain, also known as the complementarity-determining region 3 (CDR3) is an important determinant of antigen recognition. [Aversa, I. et al. (2010) Int. J. Mol. Sci. 21: 238; doi: 10.3390/ijms21072378, citing Xu, J L and Davis, M M. Immunity (2000) 13: 37-45]. The CDR3 sequence is essentially unique for each newly formed T cell, since it is highly unlikely that two T cells will express the same CDR3 nucleotide sequence [Id., citing Turner, S J et al. Nat. Rev. Immunol. (2006) 6: 883-94]. At the same time, when a T cell is activated and undergoes a clonal expansion, all the cells of the clonal lineage are equipped with an identical CDR3, which therefore acts as a natural identifier of the clonality of the lymphocytes [Id., citing Kirsch, I. et al. Mol. Oncol. (2015) 9: 2063-700].

The term “joining chain” or “J chain” as used herein refers to a small polypeptide that binds to the tail pieces of α and μ Ig heavy chains.

The term “TCR clonotype” as used herein refers to a unique nucleotide sequence that arises during the gene rearrangement process for that receptor. The combination of nucleotide sequences for the surface expressed receptor pair defines the T cell clonotype [Yassai, M B et al. Immnogenetics (2009) 61 (7): 493-502].

T cell receptors (TCRs) recognize a complex consisting of a peptide derived by proteolysis of the antigen bound to a specialized groove of a class II or class I MHC protein. The CD4+ T cells recognize only peptide/class II complexes while the CD8+ T cells recognize peptide/class I complexes. [Paul, W. E., “Chapter 1: The immune system: an introduction,” Fundamental Immunology, 4th Edition, Ed. Paul, W. E., Lippincott-Raven Publishers, Philadelphia (1999)].

The term “TCR repertoire” as used herein refers to the whole range of different TCRs present in an organism. [Aversa, I. et al. Int'l J. Molec. Sci. (2020) 21: 2378]. TCR gene sequences are markers of T-cell lineage, because TCRγ, TCRβ, and TCRα loci are sequentially rearranged during different stages of intrathymic maturation of T cells from a diverse pool of V(D)J genes. The TCRβ locus contains 47 V (TRBV), 2 D (TRBD), and 13 J (TRBJ) segments, whereas the TCRα locus comprises 42 TRAV and 61 TRAJ segments, which recombine to yield unique DNA sequences that are retained in genomes of all daughter cells. [Iyer, A. et al. Blood Adv. (2022) 6 (7): 2334-2345, citing Davis, M M and Bjorkman, PJ. Nature (1988) 334 (6181): 395-402]. This diversity of the unique DNA sequences is enhanced by the insertion of random palindromic sequences during the recombination step of V(D)J or VJ. The repertoire of normal human TCRs is in the range of 10E6 to 10E7 clonotypes [Id., citing Warren, R L., et al. Genome Res. (2011) 21 (5): 790-797; Artila, T P., et al. Science (1999) 286 (5441): 958-961; Qi, Q. et al. Proc. Nat'l Acad. Sci. USA (2014) 111 (36): 13139-13144].

The term “TACI” is an acronym for Tumor Activated Cellular Immunity.

The term “variable domain” or “V domain” as used herein refers to the domain of an Ig or TCR chain that is encoded by the corresponding variable (V) exon. The variable domains have a high degree of amino acid variability.

The term “variable exon” or “V exon” as used herein refers to the exon encoding the variable domain of an Ig or TCR protein.

The term “variable region” as used herein refers to the highly variable N-terminal portion of an Ig or TCR molecule.

The term “V(D)J recombination” or “somatic recombination” as used herein refers to site-specific recombination of pre-existing V, D and J gene segments in the Ig and TCR loci to generate unique variable (V) exons.

### Experimental

NGS Analysis of CDR3 of TCRs from RNAs Extracted from Metastatic Liver Patients Who were Treated with TATE+Anti-PD-1 Inhibitor

Sample collection: RNA samples were collected from patients before and after TATE treatment to investigate the impact of TATE in clonotypes of TCR using CDR3. Patients were treated with commercial anti-PD-1 antibody on Days 1 and 22, and TATE on Day 8. Blood sample was collected at Day 8 before TATE, and Day 22 before anti-PD-1, so that the result of CDR3 changes should be mainly due to the effect of TATE and not due to anti-PD-1 antibody.

### Methods of Analysis:

### Processing Blood Samples for MiSeq Sequencing

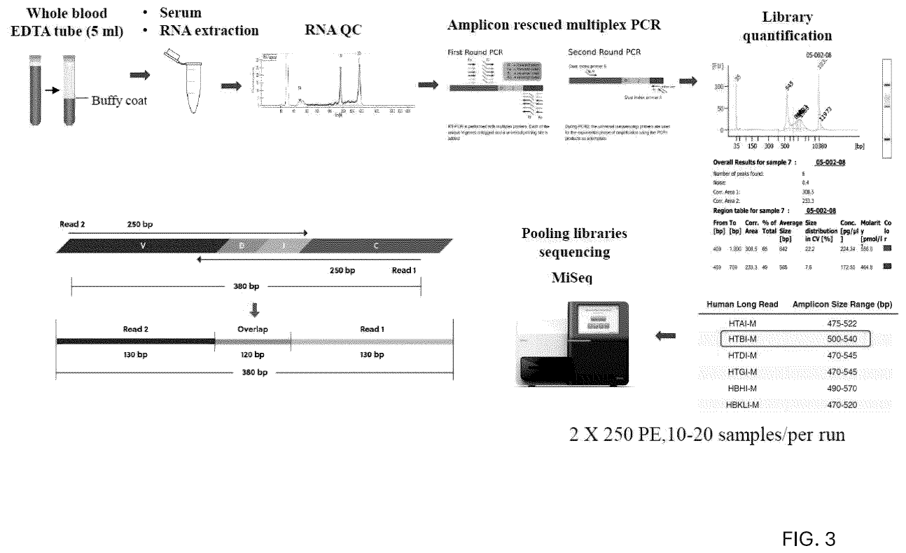

Peripheral blood was collected in lavender EDTA-containing tubes and centrifuged to collect PBMCs in a buffy coat. The cell pellet of PBMCs was incubated with RBC lysis buffer, and RNAs of the PBMCs were extracted with Trizol. After ensuring the RNA quality by a quality control check, multiplex PCR was conducted with nested PCRs using commercial primers to amplify the region from the 3′ of V region to the 5′ of C region as shown in FIG. 3. A barcode was added to allow future identification. Library quantification was conducted to evaluate the average size, concentration, molarity etc. of the amplicons, to ensure product satisfaction. The library was sequenced by a MiSeq NGS machine to collect the sequences of each amplicon in the library. Based on the PCR primer design, two reads were collected as shown in FIG. 3. Read 1 is approximately 250 bp and covers V-D-J-C with 130 bp in C for identification of C chains used. Read 2 is also ˜250 bp and covers V-D-J with 120 bp in V for identification of V chains used. There is an overlap between D-J with approximately 130 bp, which was used to determine the portion of TCR that represents the hypervariable region, or CDR3 of TCR, which encodes the fragment interacting with its cognate antigen.

### VDJ Tools for the Immune Repertoire Analysis

The following list identifies tools provided by commercial vendors for analysis of the NGS data of the CDR3 clonotypes.
Sequencing data process
Mapped read counts: V-J segments, CDR3 length, and CDR3 sequence.
V-J types and usage frequency.
CDR3 sequence and frequency
Diversity index
   Comparison of the CDR3 Reads Between Day 8 (Pre-TATE) and Day 22 (Post-TATE) in a Metastatic Liver Cancer Patient Treated with TATE Plus a Commercial Anti-PD-1 Antibody

In the pre-TATE sample, 899,319 CDR3 clones were identified. In the post-TATE sample, there were 943,221 CDR3 clones. There were 575,335 (63.97%) CDR3 clones that were present in the pre-TATE sample but disappeared after TATE. In contrast, there were 619,333 (65.67%) clones that did not exist in pre-TATE sample but newly appeared after TATE treatment. Among these 619,333 clones, 79,160 (8.39%) had a copy number above 500, and 121,709 (12.89%) had a copy number above 300.

There were 323,983 (36.03%) CDR3 clones that were present pre-TATE sample, and they are still detected in post-TATE sample, and comprised 323,888 (34.34%) clones. Although the percentages are similar, there are multiple clones showing significant expansion after TATE. For those CDR3 clones that increased by at least 10 fold, their total CDR3 copies were 1288 clones (0.14%) present in the pre-TATE sample. After TATE treatment, the total number of these same clones expanded to 35,700 (3.78%), which is a 27-fold expansion on average.

Table 1 below shows copy numbers of the unique CDR3 in a metastatic liver patient before and after TATE treatment (14 days after TATE).

| | Copy of | Copy of |
|---|---|---|
| | CDR3 pre-TATE | CDR3 post-TATE |
| Clones disappeared after TATE | 575,335 (63.97%) | — |
| Clones appeared after TATE | — | 619,333 (65.67%) |
| Newly appeared after TATE with | — | 79,160 (8.39%) |
| cDR3 copy number >500 | | |
| Newly appeared after TATE with | — | 121,709 (12.89%) |
| cDR3 copy number >300 | | |
| Exist in both Pre- and Post-TATE | 323,983 (36.03%) | 323,888 (34.34%) |
| Exist in both Pre- and Post-TATE | 1,288 (0.14%) | 35,700 (3.78%) |
| and expanded by 10 fold after | | |
| TATE | | |
| Total | 899,318 | 943,221 |

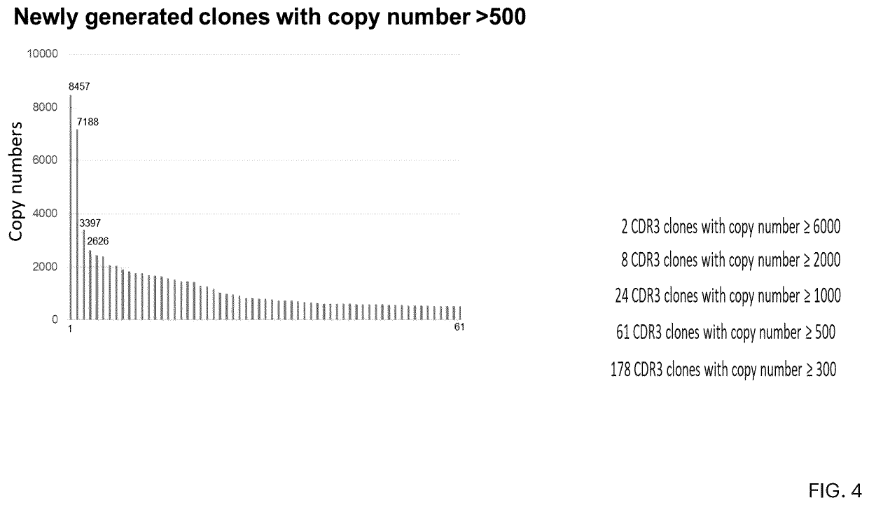

FIG. 4 is a histogram showing the clones that newly appeared after TATE treatment plotted against copy number (y-axis). Two clones had 8457 and 7188 copies, respectively, which are approximately 0.7-0.8% of total CDR3 clones. There were 178 clones with the copy number at least 300.

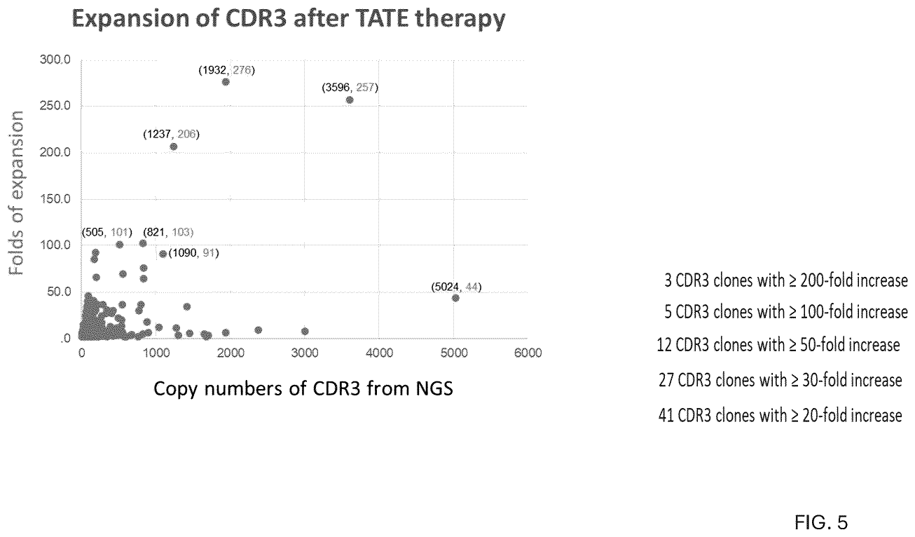

The histogram and distribution of those new CDR3 clones are shown in FIG. 5, which is a scatter plot for fold of expansion (in green, y-axis) vs. CDR3 copy numbers post-TATE (x-axis). Regarding clones that existed in both pre and post-TATE samples, there are 2,500 CDR3 clonotypes that are present in both samples. There are 27 with the copy number ratio (Day 22/Day 8) increased by 30 fold or higher, 41 with the ratio increased by 20 fold or higher. The top three ratios were 276-, 257- and 206-fold increase; and their copy numbers are 1932, 3596 and 1237, respectively, in the post-TATE sample. There are 5 with a ratio above 100 and 12 with a ratio above 50 after TATE treatment. This pattern in this metastatic liver cancer patient implies that TATE induced a strong immunization effect with multiple new clones of T cells generated or expansion of existing anti-tumor T cell clones after TATE treatment.

Although what tumor neoantigens these clones recognized remains to be determined, based on the fact that they were newly generated or expanded after a single therapeutic intervention of TATE-induced tumor necrosis, we believe that a majority of them would be targeting certain tumor neoantigens and potentially contributing to a clinical response, particular an abscopal effect.

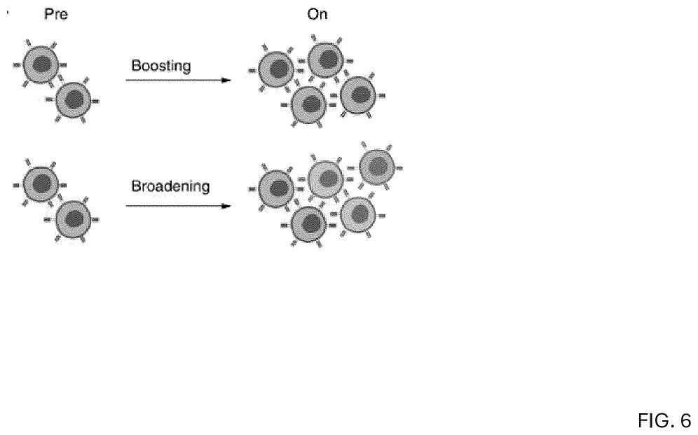

FIG. 6 is a schematic illustration depicting the effect of expansion of peripheral blood mononuclear cells after TATE and subsequent ex vivo expansion.

TACI-Expansion of the Peripheral Blood Mononucleated Cells (PBMCs) after TATE for Ex Vivo Expansion to Treat Cancer Patients Who Received TATE+Anti-PD-1 Rationale of TACI:

TATE therapy, a type of TUNIA, is able to induce tumor necrosis and convert a tumor into a therapeutic vaccine to enhance the efficacy of anti-PD-1. The synergy between TATE and anti-PD-1 has been demonstrated by frequent observation of an abscopal effect in patients receiving the combination. The mechanism of the abscopal effect is due to the TATE-induced expansion of anti-tumor T cells, which are activated by anti-PD-1 antibody to target the extra-hepatic tumor lesions not treated with TATE. A translational study described above is ongoing comprising collecting peripheral blood mononuclear cells (PBMC) for RNA extraction and next generation sequencing (NGS) analysis of the CDR3 of TCR. It is expected that clonal expansion of TCRs will occur and be confirmed by the information of the CDR3 of the anti-tumor T cells before and after TATE treatment.

We anticipate that the clonally expanded anti-tumor T cells could be either anti-tumor effector T cells converted from existing central memory cells or effector memory cells after exposure to the necrotic tumor vaccine, or T cell populations newly primed by the tumor antigen presented by macrophages or dendritic cells. Because of the nature of this new expansion or generation, the anti-tumor T cells are expected to be more active and with a high proliferating capability than other non-anti-tumor T cell populations, which are naïve or central memory cells without stimulation by their cognate antigen. If we conduct an expansion process for all T cells, the population of effector T cells with TCRs targeting tumor cells is expected to be preferentially expanded and in an increased population that could be suitable for treating the same patient. No genetic manipulation would be necessary during the process.

### Rationale

If we conduct an ex vivo expansion of the PBMCs from the peripheral blood of the patients who have been treated with TATE+anti-PD-1 and infuse these expanded PBMCs back to the patient, the expanded anti-tumor T cells would induce a clinical response against the residual tumor or even eradicate the cancer and prevent future recurrence. The high proportion of new clones plus the expanded clones suggest that a polyclonal expansion is a better approach than developing individual CAR-T cells, which are monoclonal or oligoclonal, to mitigate the risk of tumor recurrence.

The expanded T cells can be infused back to the same patient to enhance clinical response in cancer in a clinical trial.

Methods for ex vivo expansion and estimation of the cell counts
1. Isolation of PBMCs from all patients who are treated with TATE+anti-PD-1 for at least two months (TATE×2+anti-PD-1×3). RNA is extracted for NGS for cluster analysis to demonstrate the clonal expansion.
2. Collection of 20 mL of PBMCs at day 64 after TATE+anti-PD-1 for PBMC isolation by centrifugation. The normal range for WBC count is between 4,000 and 11,000/μL. Assuming 5000/μL and that 20% are lymphocytes, the total number of lymphocytes will be 1000/μL, and 20 mL blood will have 1000×1000×20=20 million cells. Assuming the yield is 20% from isolation, we anticipate we should have 4 million lymphocytes after isolation.
3. Save an aliquot of T cells (approximately 10E5 cells) and store in Trizol for RNA isolation for future NGS-pre-expansion samples. Do flow cytometry analysis of the samples with the markers CD3, CD4, CD8, CD45RO, CCR7, and CD56 (NK marker) to characterize the % of cell populations regarding naïve memory cells, central memory cells, effector memory cells, effector cells, NK cells and NK-T cells.
4. Put the isolated T cells in tissue culture chambers with commercial T cell expansion medium (for example, ImmunoCult™ from Stem Cell Technology) and grow in 5% CO2, 37° C. Do cell count at days 2, 5 and 8, and collect samples at approximately days 5, 7 and 10. Do flow cytometry analysis of these samples similar to those tested for the pre-expansion samples. Each sample should also have an aliquot for Trizol and RNA extraction for NGS analysis.

The expanded T cells can produce cytokines (e.g., IFN-gamma and IL-4) upon restimulation. According to the information brochure provided by the manufacturer, the cumulative fold of expansion could reach nearly 200 fold after 10 days of culture. The distribution of the expanded population is expected to skew toward central memory T cells (CD45 RO+ CCR7+) and effector memory T cells (CD45RO+ CCR7−) populations based on experience with CAR-T production. The distribution of the CDR3 of the TCR in the expanded populations will be established by NGS analysis.

Advantages of TACI Vs. TIL (Tumor Infiltrated Lymphocytes) and CAR-T (Chimeric Antigen Receptor-T) Therapy

Currently there are two main types of cell therapy approaches approved by FDA: ex-vivo expanded tumor-infiltrating lymphocytes or TILs and genetically engineered chimeric antigen receptor-containing T cells (CAR-T)/TCR-T (meaning T cell receptor engineered T cells) to target antigens. We compare the pros and cons of each of them with TACI. TACI is suitable for all solid tumors, there is no need for lymphodepletion, no biopsy required, no need for identification of tumor antigen, a polyclonal targeting and no genetic modification required. These advantages imply that TACI is superior to TILs and CAR-T. Two financial advantages of TACI are (1) no lymphodepletion, which uses high dose Cytoxan and Fludarabine and is associated with neutropenia and a hospital stay for at least 2 weeks until the patient's white count recovers. This course generally costs each patient or payers at least $200,000 and patients are subject to the risk of infection or sepsis during the period of neutropenia. (2) the manufacturing process of TACI is simple and straightforward, with harvested PBMCs cultured in the medium without any further manipulation, which reduces manual cost and contamination. It is anticipated that the cost of goods could be under $100,000, much lower than those for CAR-T and TILs. So combined, there could be a saving of nearly half a million dollars for each TACI autologous cell therapy.

Table 2 below provides a summary of the advantages/disadvantages of these therapies.

| | TATE/THIANA → | | |
|---|---|---|---|
| | TACI | TIL | CAR-T or TCR-T |
| Indications | Potentially all solid | Melanoma now, | Lymphoma and |
| | tumors | potentially all solid | myeloma. Limited solid |
| | | tumors | tumors with identifiable |
| | | | targets. |
| Lymphodepletion | No need, out-patient, | Required, admission | Required, admission |
| (Cytoxan + | low infection risk | ~2 weeks, 50% with | ~2 weeks, 50% with |
| Fludarabine) | | infection ($200,000. | infection ($200,000). |
| Cell source | PBMCs | Tumor biopsy | PBMCs with CAR |
| | | | vector |
| Targeted tumor | No need to identify | No need to identify | Target required |
| antigen | | | |
| Clonality | Polyclonal | Polyclonal | Mono- or oligo-clonal |
| Genetic | None | None | CAR-T or TCR-T |
| modification | | | vector |
| Manufacture | Simple expansion and | Complicated and | Complicated and |
| efforts | low cost, cost of goods | expensive, | expensive, |
| and cost | estimated <$100,000. | currently >$500,000. | currently >$400,000. |

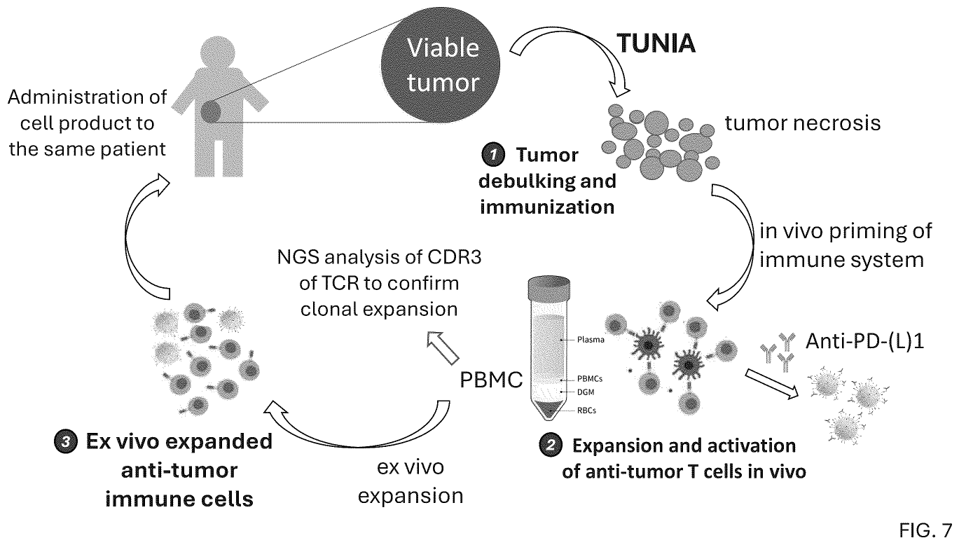

FIG. 7 depicts the whole process of the described autologous cell therapy platform approach for solid tumor patients comprising (1) inducing tumor necrosis after TUNIA leading to clonal expansion of anti-tumor T cells in blood, (2) administering anti-PD-1 therapy to produce activated T cells, (3) expanding the PBMCs ex-vivo and infusing the expanded autologous cell product back to the same patient.

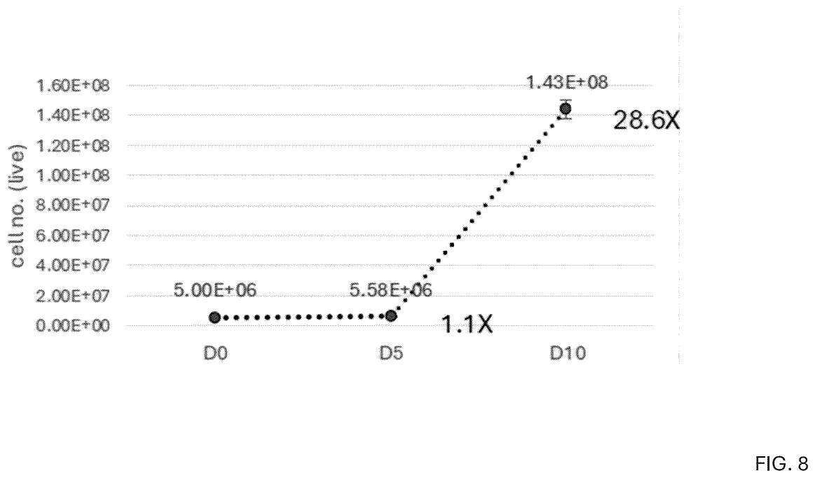

FIG. 8 is a plot of cell number vs time that shows the expansion of cells after ex vivo expansion for 10 days. The total cell count was 5.0×10E6 at initial inoculation and became 5.58×10E6 after 5 days of culture. Then the cell growth entered into a rapid proliferation phase and achieved 1.43×10E8 or a 28.6-fold expansion compared to the starting cell count.

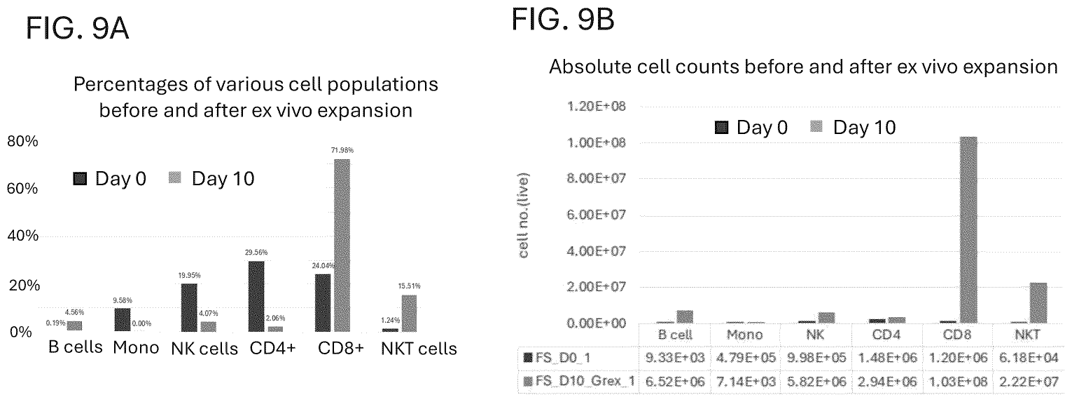

FIG. 9A and FIG. 9B show the distribution of various subpopulations of PBMCs for a patient previously treated with TUNIA before and after our ex vivo expansion. Flow cytometry analysis was conducted to determine the percentage of B cells, monocytes, NK cells, CD4+, CD8+ and NKT cells using their specific markers. FIG. 9A is a bar graph plotting cell type versus percentage of various cell populations before (day 0) and after (day 10) ex vivo expansion. It shows that three populations of cells, monocytes, NK cells and CD4 cells exhibited a dramatic reduction in their percentages before and after the process of ex vivo expansion. In contrast, B cells, CD8+ and NKT cells showed significant expansion. Without being limited by theory, the mechanism of this preferential expansion in these cell populations could be due to the immunization effect in vivo of TUNIA, since ex vivo expansion of healthy donors' PBMCs without TUNIA treatment will hardly change the percentages of distribution of cell subpopulations. Consistent with the expansion of CDR3 clones described in TABLE 1 and FIG. 4, more tumor-specific cytotoxic T cells were generated in vivo, which are also in a more active proliferative stage than the non-tumor targeted T cells. We observed that the ex vivo process resulted in a preferential expansion of the active proliferating T cells during culture. After 10 days of ex vivo expansion, CD8+ cells became the dominant cell population comprising about 72.0%, whereas NKT cells are the second most prominent population, comprising about 15.5%. FIG. 9B shows the absolute cell count of each population before (day 0) and after (day 10) ex vivo expansion. FIG. 9B shows that the CD8+ dominance in the product was even more prominent. This pattern of cell distribution is considered a highly desirable cell profile, as CD8+ population includes most cytotoxic T cells against tumors, and NKT cells are also expected to play an important role against tumors. In contrast, CD4+ cells contain the Treg population, which commonly has an immunosuppressive function to inhibit the activity of CD8+ cytotoxic T cells. The reversal of the ratio for CD4+/CD8+ in the ex vivo expanded product from the process indicated a more favorable product for anti-tumor cell therapy.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. An autologous cellular immunotherapy for treating a cancer comprising:
a. administering to a subject with a cancer (i) a Tumor Necrosis-Inducing Agent (TUNIA) to induce tumor necrosis and reduce tumor burden and (ii) a checkpoint inhibitor;
b isolating peripheral blood mononuclear cells (PBMCs) from peripheral blood of the subject by density gradient centrifugation, the PBMCs comprising a CD4+ T cell subpopulation, a CD8+ T cell subpopulation, a natural killer (NK) cell subpopulation, and an NK-T cell subpopulation;
c. extracting RNA and DNA from the CD4+ T cell subpopulation and CD8+ T cell population;
d. preparing a sequencing library from each of the RNA or DNA sample by (1) amplification to yield a pool of appropriately sized target sequences; and (2) the addition of sequencing adapters that later will interact with a next generation sequencing (NGS) platform;
e. amplifying the sequence library by polymerase chain reaction (PCR) to yield a library comprising a collection of specifically sized DNA fragments;
f. loading the library onto a sequencer and performing parallel sequencing using a next generation sequencing (NGS) platform;
g. after sequencing is complete, filtering the reads for quality, amplicon size, and agreement between paired ends;
h. assembling and aligning the reads to a reference genome for a T cell receptor comprising 2 protein chains;
i. identifying expanded clonal variants of complementarity-determining region-3 (CDR3) of the T cell receptor comprising 2 protein chains by comparing the reads (assembled or raw) to the sequence of the CDR3 of a reference TCR sequence or to reads from another sample to identify variants;
j. expanding in vitro the PBMCs from the peripheral blood of the patient comprising the expanded clonal variants of CDR3; and
k. administering to the subject by infusion the PBMCs comprising a polyclonal expanded CDR3 T cell response in (j).

2. The autologous cellular immunotherapy of claim 1, wherein the administering is for at least two months.

3. The autologous cellular immunotherapy of claim 1, further comprising flow cytometry analysis of a sample of the PBMCs in step (b) and in step (j) after expansion of the PBMCs with CD3, CD4, CD8, CD45RO, CCR7, and CD56 markers.

4. The autologous cellular immunotherapy of claim 3, wherein the flow cytometry analysis characterizes the cell populations comprising naïve memory cells, central memory cells, effector memory cells, effector cells, Natural Killer cells and NK-T cells in the PBMCs.

5. The autologous cellular immunotherapy of claim 1, wherein the clonal variants of CDR3 that appear after TATE treatment recognize a tumor neoantigen.

6. The autologous cellular immunotherapy of claim 1, wherein, after ex vivo expansion in step (j),
a. the percentage of the total PBMC cell population represented by each of the monocyte subpopulation, the NK cell subpopulation and the CD4 cell subpopulation was reduced compared to its percentage before ex vivo expansion; and
b. the percentage of the total PBMC cell population represented by each of the B cell subpopulation, the CD8+ cell population and the NKT cell population increased compared to its percentage before ex vivo expansion.

7. The autologous cellular immunotherapy of claim 6, wherein after ex vivo expansion for at least 10 days, the CD8+ cell subpopulation comprising cytotoxic T cells and the NKT cell subpopulation dominate the PBMC cell population while the CD4+ cell subpopulation comprising an immunosuppressive Treg subpopulation is reduced compared to its percentage of the total PBMC cell population before ex vivo expansion.

8. The autologous cellular immunotherapy of claim 6, wherein the tumor necrosis-inducing agent (TUNIA) step (a) comprises an in vivo immunizing step.
